(19) United States
(12) Patent Application Publication
(10) Pub. No.: US 2024/0390419 A1
(43) Pub. Date: Nov. 28, 2024

(54) CD3 EPSILON CHIMERIC ANTIGEN RECEPTORS AND METHODS OF USE THEREOF

(71) Applicant: The Regents of the University of Colorado, A Body Corporate, Denver, CO (US)

(72) Inventors: Mark Eric KOHLER, Denver, CO (US); Terry J. FRY, Bethesda, MD (US); Catherine DANIS, Denver, CO (US); Lillie LEACH, Denver, CO (US); Michael Eric YARNELL, Denver, CO (US)

(21) Appl. No.: 18/684,037

(22) PCT Filed: Aug. 26, 2022
(86) PCT No.: PCT/US2022/075511

§ 371 Date: Feb. 15, 2024

Related U.S. Application Data
(60) Provisional application No. 63/321,551, filed on Mar. 18, 2022.
(60) Provisional application No. 63/237,358, filed on Aug. 26, 2021.

(51) Int. Cl. A61K 35/17; A61K 39/00; A61P 35/02; C07K 16/28; C12N 5/0783

(52) U.S. Cl. CPC A61K 35/17; A61K 39/4611; A61K 39/4631; A61K 39/464412; A61K 39/464413; A61P 35/02; C07K 16/2803; C12N 5/0636; A61K 2239/13; A61K 2239/21; A61K 2239/22; A61K 2239/48

(57) ABSTRACT

The disclosure describes T cells that express chimeric antigen receptors (CARs), as well as pharmaceutical compositions comprising T cells and methods of making and using such T cells. Particularly, this disclosure describes T cells expressing a first CAR that binds to a first antigen and a second CAR comprising a CD3ε intracellular signaling domain that binds to a second antigen, and methods of use in treating cancers, such as solid tumors and hematological malignancies.

### RELATED APPLICATIONS

This application claims priority to, and the benefit of, U.S. Provisional Patent Application No. 63/321,551, filed on Mar. 18, 2022, and U.S. Provisional Patent Application No. 63/237,358, filed on Aug. 26, 2021, each of which is incorporated herein by reference in its entirety.

### GOVERNMENT SUPPORT

This invention was made with government support under Grant No. K12CA086913-20 awarded by the National Institutes of Health. The government has certain rights in the invention.

### FIELD OF INVENTION

The present invention relates generally to the fields of molecular biology, immunology, oncology and medicine. More particularly, it concerns immune cells expressing chimeric antigen receptors, such as chimeric antigen receptors that bind to a target protein.

### INCORPORATION BY REFERENCE OF SEQUENCE LISTING

The Sequence Listing XML associated with this application is provided electronically in XML file format and is hereby incorporated by reference into the specification. The name of the XML file containing the Sequence Listing is “UNCO-042_001WO_SeqList_ST26.xml”. The XML file is about 137 KB, was created on Aug. 25, 2022, and is being submitted electronically via USPTO Patent Center.

### BACKGROUND OF THE INVENTION

Over the past decade, Chimeric Antigen Receptor (CAR) T cell therapy has demonstrated remarkable efficacy against B-lineage leukemias, lymphomas and multiple myeloma and held promise for the treatment of all malignancies which are otherwise incurable with conventional therapies. Across multiple clinical trials, CAR T cells targeting the CD19 antigen have induced complete remission in 70-90% of patients with multiply-relapsed and/or refractory acute lymphoblastic leukemia (ALL). This remarkable upfront success does not, however, translate to long term remissions for many patients as longitudinal studies have demonstrated that less than 50% of CAR T cell treated patients remain in remission beyond 1 year after therapy due to post-CAR relapses. Post-CAR relapses present a clinical challenge as conventional chemotherapy, antibody-based therapies (blinatumomab and inotuzumab) and retreatment with the same CAR T cells have been found to infrequently be capable of reinducing patients into remissions, the majority of which were short-lived.

CD19-directed CAR T cell therapy for relapse and/or refractory B-lineage lymphomas has demonstrated similar results, with Objective Response Rates (ORR) of 52-82%, and 40-54% of patients achieving a Complete Response (CR), yet disease recurrence and/or progression after CAR T cell therapy remains common with less than 40% of patients remaining progression-free 1 year later. Consistent with the experience in leukemia, there are no established therapies which are effective for lymphoma patients whose disease relapsed and/or progressed after CAR T cells and reinfusion of the same CAR T cells has been largely ineffective.

Relapses after CAR therapy occur through a variety of mechanisms. In B cell leukemias treated with CD19-directed CAR T cells, upfront treatment failures and relapses in which the leukemia continues to express the CD19 antigen are highly correlated to low levels of CAR T cell expansion and a short duration of CAR T cell persistence in the patient, and it is generally held that improving CAR T cell expansion and persistence would improve outcomes by preventing relapses of antigen-positive leukemias. Another major mechanism of relapse after CAR T cell therapy is the modulation of the targeted antigen on the malignant cells as a means of escaping CAR T cell detection. In B cell leukemias, this has been mostly observed as the emergence of CD19-negative leukemia cells upon relapse. Similarly, decreased surface expression of the CD19 antigen on B-lineage lymphomas has been implicated in refractoriness to and relapse after treatment with CD19-directed CAR T cells. In either antigen-loss or down-modulation current CAR T cell therapies directed at CD19 are ineffective, an outcome which has been generalizable to other CAR-targeted antigens beyond CD19.

To overcome antigen-modulated relapses in leukemia/lymphoma CARs have been developed to target alternative antigens. CD22-directed CAR T cells have demonstrated the ability to induce remissions in 70-80% of patients with ALL, including patients with CD19-negative relapses after immunotherapy. Unfortunately, relapse after CD22-directed CAR T cell therapy was frequently observed in patients, due largely to down-regulation of the CD22 antigen. Currently, CD22 CAR T cell therapy is being used to bridge patients to a consolidative hematopoietic stem cell transplant (HSCT), however the long-term outcomes of this strategy are not yet known and many patients may be ineligible due to significant co-morbidities, prior HSCT(s) or a lack of a suitable donor. Thus, the clinical utility of CD22-directed CAR T cells is limited by the inability to target malignant cells expressing low-levels of antigen, similar to CD19 CAR T cell experience in lymphoma and likely representing a fundamental problem for any therapy targeting an antigen using T cells (or other immune effector cells) expressing a 2^{nd} generation CAR.

Thus, there is a need in the art for alternative approaches for generating genetically engineered immune cells (e.g. T cells) that are useful as therapeutics. There exists a need for new strategies to mitigate relapse after CAR T cell therapy to improve patient outcomes by enhancing the persistence and antigen-sensitivity of CAR T cells to improve the clinical efficacy of CAR T cell therapy against a variety of antigens and malignancies. The present invention addresses these unmet needs in the art.

### SUMMARY OF INVENTION

The present disclosure provides genetically modified immune cells comprising: a) a first chimeric antigen receptor (CAR) comprising an antigen binding domain that binds to a first antigen, a transmembrane domain and an intracellular signaling domain; and b) a second CAR comprising an antigen binding domain that binds to a second antigen, a transmembrane domain and a CD3ε intracellular signaling domain. In some aspects, the first antigen and the second antigen are different. In some aspects, the first antigen and the second antigen are the same.

In some aspects, the intracellular signaling domain of the first CAR comprises a CD3zeta intracellular signaling domain. In some aspects, the CD3zeta intracellular signaling domain comprises the amino acid sequence of SEQ ID NO: 25 or SEQ ID NO: 26, preferably wherein the CD3zeta intracellular signaling domain comprises the amino acid sequence of SEQ ID NO: 25.

In some aspects, the intracellular signaling domain of the first CAR further comprises at least one additional intracellular signaling domains selected from the group consisting of a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8a intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain and any combination thereof. In some aspects, the at least one additional intracellular signaling domain is a 4-1 BB intracellular signaling domain comprising the amino acid sequence of SEQ ID NO: 18.

In some aspects, the CD3ε intracellular signaling domain of the second CAR comprises the amino acid sequence of SEQ ID NO: 27 or SEQ ID NO: 28, preferably wherein the CD3ε intracellular signaling domain of the second CAR comprises the amino acid sequence of SEQ ID NO: 28.

In some aspects, the transmembrane domain of the first CAR and/or the second CAR is derived from a transmembrane domain selected from the group consisting of a CD8a transmembrane domain, a CD28 transmembrane domain, a CD3z transmembrane domain, a CD3ε transmembrane domain, a CD4 transmembrane domain, a 4-1BB transmembrane domain, a OX40 transmembrane domain, a ICOS transmembrane domain, a PD-1 transmembrane domain, a LAG-3 transmembrane domain, a 2B4 transmembrane domain, a BTLA transmembrane domain and any combination thereof. In some aspects, the transmembrane domain of the first CAR is derived from a CD8alpha transmembrane domain comprising the amino acid sequence of SEQ ID NO: 13. In some aspects, the transmembrane domain of the second CAR is derived from a CD3ε transmembrane domain comprising the amino acid sequence of SEQ ID NO: 15.

In some aspects, the antigen binding domain of the first CAR and/or the antigen binding domain of the second CAR is an antibody, an antibody fragment, a single chain antibody, a single domain antibody, an scFv, a VH or a VHH or antigen binding fragment thereof. In some aspects, the antigen binding domain of the first CAR and/or the antigen binding domain of the second CAR further comprises a leader domain selected from the group consisting of a CD8a leader domain and a IgG signal peptide domain. In some aspects, the leader domain is a CD8a leader domain comprising the amino acid sequence of SEQ ID NO: 1 or SEQ ID NO: 2. In some aspects, the leader domain is a IgG signal peptide domain comprising the amino acid Sequence of SEQ ID NO: 4.

In some aspects, the first antigen and the second antigen are a tumor associated antigens. In some aspects, wherein the first antigen and the second antigen is a tumor associated antigen, the tumor associated antigen is selected from a group consisting of CD19, CD22, CD20, CD138, BCMA, CD33, CD123, FLT, CLL, CD56, CD34, CD117, CD14, CD133, CD44v6, CD47, CD64, CD96, CD97, CD99, CD45, CD9, Muc1, Lewis-Y, IL1RAP, FR-beta, CD5, CD7, CD38, CD30, B7-H3, HER2, CD44v6, CEA, c-Met, EGFRvIII, Epcam, EphA2, FR-alpha, GD2, GPC3, IL13R-alpha2, IL11R-alpha, L1-CAM, mesothelin, MUC1, MUC16, NKGD2 and PSCA. In some aspects, the first antigen is CD22. In some aspects, the second antigen is CD19. In some aspects, the second antigen is a CD22. In some aspects, the first antigen is a CD22 and the second antigen is a CD19. In some aspects, the first antigen is a CD22 and the second antigen is a CD22.

In some aspects, the immune cells are T-cells, Natural Killer (NK) cells, Natural Killer (NK)-like cells, Cytokine Induced Killer (CIK) cells, hematopoietic progenitor cells, peripheral blood (PB) derived T cells or umbilical cord blood (UCB) derived T-cells. In some aspects, the immune cell is a T-cell.

In some aspects, the first CAR comprises the amino acid sequence of SEQ ID NO: 57, 86, 98 or 100. In some aspects, the second CAR comprises the amino acid sequence of SEQ ID NO: 59, 90, or 103. In some aspects, the first CAR comprises the amino acid sequence of SEQ ID NO: 86 and the second CAR comprises the amino acid sequence of SEQ ID NO: 59. In some aspects, the first CAR comprises the amino acid sequence of SEQ ID NO: 98 and the second CAR comprises the amino acid sequence of SEQ ID NO: 90.

The present disclosure provides a composition comprising the genetically modified immune cells of the present disclosure and a pharmaceutically acceptable carrier.

The present disclosure provides a composition comprising a population of cells, wherein the plurality of cells of the population comprises the genetically modified immune cells of the present disclosure. In some aspects, the plurality of the cells of the population comprises at least 1%, 2%, 3%, 4%, 5%, 6%, 7%, 8%, 90%, 10%, 15%, 20%, 25%, 30%, 35%, 40%, 45%, 50%, 55%, 60%, 65%, 70%, 75%, 80%, 85%, 90%, 95%, 97%, 99% or any percentage in between of the genetically modified immune cells of the present disclosure.

The present disclosure provides a polynucleotide encoding the first CAR and the second CAR of the genetically modified immune cells of the present disclosure. In some aspects, a nucleic acid sequence encoding a self-cleaving peptide sequence is located in between the nucleic acid sequence encoding the first CAR and the nucleic acid sequence encoding the second CAR. In some aspects, the self-cleaving peptide sequence comprises the amino acid sequence of SEQ ID NO: 67. In some aspects, the first CAR and the second CAR encoded on a single vector. In some aspects, the vector is a viral vector, a lentivirus vector, a non-viral vector or a transposon. In some aspects, the vector is a bicistronic lentiviral vector.

The present disclosure provides methods of producing a population of genetically modified immune cells, comprising: a) introducing into a plurality of immune cells a composition comprising a polynucleotide sequence of the present disclosure, thereby generating a population of genetically modified immune cells; b) culturing the population of genetically modified immune cells under conditions suitable for integration of the polynucleotide sequence; c) expanding and/or selecting at least one cell from the population of genetically modified immune cells that expresses the first CAR and the second CAR on the cell surface.

The present disclosure provides methods of treating cancer in a subject in need thereof comprising administrating a composition of the present disclosure. In some aspects, the administration of a composition comprising a modified immune cell comprising first CAR and the second CAR increases the immune response against a target cell in comparison to the administration of a composition comprising a modified immune cell comprising a first CAR alone. In some aspects, the increased immune response at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between greater than a composition comprising a modified immune cell comprising a first CAR alone.

In some aspects, the cancer is a solid tumor, a B cell malignancy, a myeloid malignancy, a T-cell malignancy, acute lymphoblastic leukemia, acute lymphoblastic lymphoma, Non-Hodgkin lymphoma, Hodgkins lymphoma, chronic lymphocytic leukemia, multiple myeloma, acute myeloid leukemia, myelodysplastic syndrome, myeloproliferative neoplasms, chronic myeloid leukemia, T lymphoblastic leukemia, T lymphoblastic lymphoma or Anaplastic Large Cell Leukemia. In some aspects, the cancer has a low cell surface expression of the first antigen and/or a low cell surface expression of the second antigen.

### BRIEF DESCRIPTION OF THE DRAWINGS

FIG. 1 is a schematic depicting a standard 2^{nd} generation CAR targeting the CD22 antigen (“2G-22BBz CAR”) (left panel) and an exemplary bicistronic CAR construct of the present disclosure (“22×19CD3e CAR”) (right panel). In the “22×19CD3e CAR”, the first CAR targets the CD22 antigen (left CAR on the right panel) and the second CAR targets CD19 (right CAR on the right panel). An exemplary 2G-22BBz CAR comprises the amino acid sequence of SEQ ID NO: 86. An exemplary 22×19CD3e CAR comprises the amino acid sequence of SEQ ID NO: 88.

FIGS. 2A-2B are a series of images and a graph showing bioluminescent imaging (BLI) analysis of mice engrafted with 10⁶ NALM6 cells and subsequently treated with exemplary bicistronic 22×19CD3e (also referred to as “CD22×CD19-CD3e”) CAR T cells, standard second generation 2G-22BBz CAR (also referred to as “CD22 CAR”) T cells and no treatment (No Tx) controls. Days post CAR T cell infusion is depicted along the y-axis. The presence of NALM6 cells per mouse was determined via bioluminescent imaging as depicted in the scale (right). FIG. 2A shows BLI imaging of mice. FIG. 2B is a graph showing the quantitation of total flux (p/s) of each group of mice from FIG. 2A. The x-axis depicts the days post-injection with CAR T cells.

FIGS. 3A-3C are a series of graphs depicting the ability of exemplary bicistronic 22×19CD3e CAR T cells to kill NALM6 leukemia cells in vitro compared to standard 2G-22BBz CAR T cells. CAR T cells and various NALM6 cells were co-cultured for 16 hour at various ratios of Effector (CAR T cells) to Targets (NALM6 cells) (E:T Ratio), as indicated on the x-axis of each graph. FIG. 3A shows lysis of CD22/CD19-Negative NALM6 cells. FIG. 3B shows lysis of CD22-Low NALM6 cells. FIG. 3C shows lysis of CD22-High NALM6 cells.

FIG. 4 is a series of graphs depicting the ability of exemplary bicistronic 22×19CD3e CAR T cells to kill NALM6 leukemia cells expressing either CD22 alone (top right panel), CD19 alone (bottom left panel), CD22 and CD19 (bottom right panel), or neither CD22 nor CD19 (top left panel) compared to standard 2G-22BBz CAR T cells. CAR T cells (Effector; E) and NALM6 cells (Target; T) were co-cultured for 16 hour at various ratios (E:T Ratio), as indicated on the x-axis of each graph. The specific lysis (%) of NALM6 cells was quantified as depicted on the y-axis.

FIG. 5 is a graph showing the persistence of CAR T cells in bone marrow taken from mice treated with bicistronic 22×19CD3e CAR T cells compared to standard 2G-22BBz CAR T cells. Cells from the bone marrow were analyzed by flow cytometry. The y-axis depicts the percentage of viable cells that are identified as CAR T cells.

FIG. 6 is a series of pie charts demonstrating the phenotype of 22×19CD3e CART cells compared to standard 2G-22BBz CAR T cells in two separate donors (Donor 13 and Donor 14). CAR T cell products were analyzed by flow cytometry for expression of CAR, CD4, CD8, CD62L, CD45RA and CD95. (TSCM, stem memory T cells; TCM, central memory T cells; TEM, effector memory T cells; TEMRA, terminally differentiated effector memory T cells.)

FIG. 7 is a schematic depicting a standard 2^{nd} generation CAR targeting the CD22 antigen (“2G-22BBz CAR”) (left panel) and an exemplary bicistronic CAR construct (“22×22CD3e CAR”) (right panel). In the 22×22CD3e CAR, both the first CAR (left CAR of right panel and second CAR bind to a CD22 antigen. An exemplary 2G-22BBz CAR comprises the amino acid sequence of SEQ ID NO: 86. An exemplary 22×22CD3e CAR comprises the amino acid sequence of SEQ ID NO: 95.

FIG. 8 is a series of graphs depicting the ability of exemplary bicistronic 22×22CD3e CAR T cells (“22×22CD3e”) to kill NALM6 leukemia cells expressing high or low levels of CD22 in vitro compared to standard 2G-22BBz CAR T cells (2G-22BBz) and untransduced T cell negative controls (Mock).

FIGS. 9A-9B are a series of charts and graphs showing the phenotype of bicistronic 22×22CD3e CAR T cells compared to standard 2G-22BBz CAR T cells. FIG. 9A shows that 22×22CD3e CAR T cells contain more CD4+ T cells with a TSCM phenotype compared to CD4+2G-22BBz CAR T cells. (TSCM, stem memory T cells; TCM, central memory T cells; TEM, effector memory T cells; TEMRA, terminally differentiated effector memory T cells.) FIG. 9B shows levels of IL7RA on both CD4+ and CD8+ 22×22CD3e CAR T cells compared to 2G-22BBz CAR T cells.

### DETAILED DESCRIPTION OF THE INVENTION

The present invention generally provides cells, including immune cells (e.g., T cells, B cells, Natural Killer (NK) cells, monocytes, macrophages or artificially generated cells with immune effector function) derived from a patient, a healthy donor, a differentiated stem cell (including but not limited to induced pluripotent stem cells (iPSC), embryonic stem cells, hematopoietic and/or other tissue specific stem cells) or a non-human source, which are genetically modified to express a first antigen recognizing receptor (e.g., chimeric antigen receptor (CAR)) that binds to a first antigen along with a second antigen recognizing receptor (e.g., CAR) comprising a CD3; intracellular signaling domain that binds to a second antigen, and methods of use thereof for the treatment of cancer, infection, autoimmunity, alloimmunity, lymphoproliferative disease, pathologic immune dysregulation and other pathologies where an increase in an antigen-specific immune response is desired or for the facilitation of solid organ or hematopoietic stem cell transplantation. The first CAR and the second CAR may recognize an identical epitope or different epitopes on the same antigen, or epitopes found on two distinct antigens. Immune cell (e.g. T cell) activation is mediated by engagement of either the first CAR to its cognate antigen (e.g., CD22) or the second CAR comprising a CD3ε intracellular signaling domain to its cognate antigen (e.g., CD19) with signal amplification leading to enhanced persistence, antigen-sensitivity and efficacy occurring when both the first and second CARs are simultaneously engaged to their respective cognate (e.g., CD22 and CD19).

CARs, which are at times referred to as artificial T cell receptors, chimeric T cell receptors (cTCR), T-bodies or chimeric immunoreceptors, are engineered receptors now well known in the art. They are used primarily to transform immune effector cells, in particular T cells, to provide those cells with a desired antigen specificity and effector response. Adoptive cell therapies using CAR-T cells are particularly under investigation in the field of cancer therapy. In these therapies, T cells are removed from a patient, donor or are derive from a stem cell source and engineered to express CARs specific to the antigens found in a particular form of cancer. The CAR-T cells, which can then recognize and kill the cancer cells, are reintroduced into the patient whereupon the CAR T cells undergo proliferative expansion, elimination of target antigen-positive cells and, in a minority of patients, transition to a long-lasting, persistent population with retained anti-tumor effector activity.

First generation CARs provide a TCR-like signal from an Immunoreceptor Tyrosine-based Activation Motif (ITAM) containing intracellular signaling domain, most commonly derived from the CD3 zeta (CD3z) molecule, and thereby elicit tumoricidal functions. However, the engagement of CD3z-chain fusion receptors may not suffice to elicit substantial IL-2 secretion and/or T cell proliferation in the absence of a concomitant co-stimulatory signal. In physiological T cell responses, optimal lymphocyte activation requires the engagement of one or more co-stimulatory receptors such as CD28 or 4-1BB. In the setting of suboptimal activation elicited by first generation CARs, T cell activity in vivo is often transient and incapable of controlling the malignancy.

Second (2nd) generation CARs have been constructed to transduce a functional antigen-dependent co-stimulatory signal in human primary T cells in addition to antigen-dependent TCR-like signal, permitting T cell proliferation in addition to tumoricidal activity. Second generation CARs most commonly provide co-stimulation using co-stimulatory domains (synonymously, co-stimulatory signaling regions) derived from CD28 or 4-1BB. The combined delivery of co-stimulation plus a CD3 zeta signal renders 2nd generation CARs superior in terms of function as compared to their first generation counterparts (CD3z signal alone). An example of a 2nd generation CAR is found in U.S. Pat. No. 7,446,190, incorporated herein by reference.

Third (3rd) generation CARs have also been prepared. These combine multiple co-stimulatory domains (synonymously, co-stimulatory signaling regions) with a TCR-like signaling domain in cis, such as CD28+4-1BB+CD3z or CD28+OX40+CD3z, to further augment potency. In the 3rd generation CARs, the co-stimulatory domains are aligned in series in the CAR endodomain and are generally placed upstream of CD3z or its equivalent. In general, however, the results achieved with these third generation CARs have been disappointing, showing only a marginal improvement over 2nd generation configurations, with some 3rd generation CARs being inferior to 2nd generation configurations.

This present invention is the first to utilize a first CAR (i.e. a 1st generation, a 2nd generation or a 3rd generation CAR) in conjunction with a second CAR having the intracellular signaling domain of CD3ε (“CD3 epsilon” or “CD3e”) as a means of amplifying CAR signaling and enhancing persistence and antigen-sensitivity. These T cells genetically engineered to express the dual CAR system demonstrate superior activity and persistence as compared to 1st generation CAR-T cells, 2nd generation CAR-T cells, and 3rd generation CAR-T cells. Thus, the present invention overcomes problems associated with current technologies by providing antigen-specific immune cells (e.g. T cells) for immunotherapy, such as for the treatment of immune-related diseases, including cancer, autoimmune disorders and infection.

The invention is based, at least in part, on the discovery that the simultaneous engagement of two antigens co-expressed by a tumor cell by a first co-stimulatory and ITAM-containing receptor and a second CD3ε-containing antigen recognizing receptor is useful for activating and stimulating an immunoreactive cell. In particular, the reactivity against cells expressing either antigen alone may be diminished relative to responses to cells expressing both antigens due to a lack of cooperative signaling, yet productive T cell activation can occur against target cells expressing even low levels of either targeted antigen. However, T cell activation in the presence of both antigens is greater than the T cell activation with either CAR alone. Thus, this approach augments the T cell reactivity against tumors expressing low levels of tumor associated antigens.

The sensitivity of CARs for their cognate antigen greatly impacts patient outcomes of those who received CAR T therapy. Multiple sub-clones of the pre-B ALL cell line, NALM6, expressing variable amounts of the CD22 antigen were generated and when the level of CD22 falls below 1500-2000 molecules per cell there is a significant decrease in CAR T cell cytokine production, cytotoxicity, effector differentiation, persistence and in vivo efficacy. The impact of antigen density on CAR T cell function is not unique to CD22 CAR T cells, as CAR T cells against CD19, CD20, HER2, ALK and B7-H3 have all been shown to have decreased activity against antigen-low targets. Furthermore, recent clinical observations have associated low levels of CD19 antigen with treatment failure and/or relapse in patients undergoing CD19-directed CAR T cell therapy for diffuse large B cell lymphoma.

While the impact of low antigen-sensitivity of CAR T cells has been described, the mechanism underlying it has not yet been elucidated. A high sensitivity to low levels of antigen is a hallmark of conventional T cells activated through their endogenous T cell receptor (TCR), with evidence of T cell activation occurring in response to fewer than 10 antigen-MHC complexes/cell and full effector responses to fewer than 200 antigen-MHC complexes/cell. The sensitivity of the TCR is due, in part, to the formation of a highly organized immune synapse and subsequently, the formation of the signalosome around a conglomerate of LAT molecules in which the signal transduction machinery of the T cell localizes at the site of antigen binding to amplify proximal signaling events and activate multiple divergent downstream signaling pathways. CARs, conversely, do not form well-organized immune synapses in which to concentrate the necessary components of the signalosome to the site of receptor-activation within a cell. The disorganization of the CAR immune synapse and subsequent inefficient assembly and utilization of the signalosome leads to suboptimal signaling within the T cell, impairing the T cell response to low levels of antigen and diminishing higher-level T cell functions, such as the establishment of a long-lived population of persistent CAR T cells in vivo.

The inability of CAR T cells to target low-levels of antigen is immediately of clinical importance, as this is the major mechanism for relapse in patients treated with CD22 CAR T cells, which is the most proven therapeutic option for patients with CD19-negative leukemia after immunotherapy. Similarly, evidence is mounting that low levels of CD19 antigen are associated with increased risk of primary treatment failure and relapse in patients with Diffuse Large B cell Lymphoma. Clinical studies of B Cell Maturation Antigen (BCMA)-directed CAR T cell have suggested that upfront efficacy of the CAR T cells is diminished in patients with multiple myeloma expressing low levels of the targeted BCMA antigen. Furthermore, reduced expression of BCMA has been commonly observed upon disease progression and/or relapse after CAR T cell therapy, further emphasizing the clinical importance of enabling CAR T cells to efficiently target antigen low malignant cells.

Accordingly the present invention provides a novel approach to addressing the shortcoming of current CAR T cell therapy by improving the ability of the T cells to recognize tumor cell that express low levels of antigen, and by increasing CAR T cell persistence, thereby improving clinical patient outcomes.

While the immune cells of the present disclosure may be targeted to any combination of antigens, exemplary antigens for the CARs disclosed herein include but are not limited to CD22 and CD19. In particular aspects, the immune cells are dually targeted to an antigen combination including but not limited to CD19 and CD20, CD20 and CD22, CD19 and CD79a, CD22 and CD79a, CD20 and CD79a, CD19 and CD79b, CD22 and CD79b, CD20 and CD79b, CD19 and CD5, CD138 and BCMA, CD38 and BCMA, CD19 and BCMA, CD19 and CD138, CD19 and GPRC5D, BCMA and GPRC5D, CD138 and GPRC5D, CD38 and GPRC5D, CD5 and CD7, CD5 and TCR alpha or beta chain, CD7 and TCR alpha or beta chain, CD5 and CD38, CD7 and CD38, CD30 and ALK, CD33 and FLT3, CD33 and CD123, CD33 and CLEC1A, CD33 and CD56, CD33 and CD34, CD33 and CD117, CD33 and CD14, CD33 and CD133, CD33 and CD44v6, CD33 and CD47, CD33 and CD64, CD33 and CD96, CD33 and CD97, CD33 and CD99, CD33 and CD16, CD33 and CD45, CD33 and CD9, CD33 and Muc1, CD33 and Lewis-Y. CD33 and IL1-RAP, CD33 and FR-beta, CD33 and ROR1, CD123 and FLT3. CD123 and CLEC1A, CD123 and CD56, CD123 and CD34, CD123 and CD117, CD123 and CD14, CD123 and CD133, CD123 and CD44v6, CD123 and CD47, CD123 and CD64, CD123 and CD96, CD123 and CD97, CD123 and CD99, CD123 and CD16, CD123 and CD45, CD123 and CD9, CD123 and Muc1, CD123 and Lewis-Y, CD123 and IL1-RAP, CD123 and FR-beta, CD123 and ROR1, FLT3 and CLEC1A, FLT3 and CD56, FLT3 and CD34, FLT3 and CD 117, FLT3 and CD14, FLT3 and CD133, FLT3 and CD44v6, FLT3 and CD47, FLT3 and CD64, FLT3 and CD96, FLT3 and CD97, FLT3 and CD99, FLT3 and CD16, FLT3 and CD45, FLT3 and CD9, FLT3 and Muc1, FLT3 and Lewis-Y, FLT3 and IL1-RAP, FLT3 and FR-beta, FLT3 and ROR1, CLEC1A and CD56, CLEC1A and CD34, CLEC1A and CD 117, CLEC1A and CD14, CLEC1A and CD133, CLEC1A and CD44v6, CLEC1A and CD47, CLEC1A and CD64, CLEC1A and CD96, CLEC1A and CD97, CLEC1A and CD99, CLEC1A and CD16, CLEC1A and CD45, CLEC1A and CD9, CLEC1A and Muc1, CLEC1A and Lewis-Y, CLEC1A and IL1-RAP, CLEC1A and FR-beta, CLEC1A and ROR1, CD56 and CD34, CD56 and CD 117, CD56 and CD14. CD56 and CD133. CD56 and CD44v6. CD56 and CD47, CD56 and CD64, CD56 and CD96, CD56 and CD97, CD56 and CD99, CD56 and CD16, CD56 and CD45, CD56 and CD9, CD56 and Muc1, CD56 and Lewis-Y, CD56 and IL1-RAP, CD56 and FR-beta, CD56 and ROR1, CD34 and CD 117, CD34 and CD14, CD34 and CD133, CD34 and CD44v6, CD34 and CD47. CD34 and CD64. CD34 and CD96, CD34 and CD97, CD34 and CD99, CD34 and CD16, CD34 and CD45, CD34 and CD9, CD34 and Muc1, CD34 and Lewis-Y. CD34 and IL1-RAP, CD34 and FR-beta, CD34 and ROR1, CD 117 and CD14, CD 117 and CD133, CD 117 and CD44v6, CD 117 and CD47, CD 117 and CD64, CD 117 and CD96, CD 117 and CD97, CD117 and CD99, CD 117 and CD16, CD 117 and CD45, CD 117 and CD9, CD117 and Muc1, CD117 and Lewis-Y, CD 117 and IL1-RAP, CD 117 and FR-beta, CD 117 and ROR1, CD14 and CD133, CD14 and CD44v6, CD14 and CD47, CD14 and CD64, CD14 and CD96, CD14 and CD97, CD14 and CD99, CD14 and CD16, CD14 and CD45, CD14 and CD9, CD14 and Muc1, CD14 and Lewis-Y, CD14 and IL1-RAP, CD14 and FR-beta, CD14 and ROR1, CD133 and CD44v6, CD133 and CD47, CD133 and CD64, CD133 and CD96, CD133 and CD97, CD133 and CD99, CD133 and CD16, CD133 and CD45, CD133 and CD9, CD133 and Muc1, CD133 and Lewis-Y, CD133 and IL1-RAP, CD133 and FR-beta, CD133 and ROR1, CD44V6 and CD47, CD44V6 and CD64, CD44V6 and CD96, CD44V6 and CD97, CD44V6 and CD99, CD44V6 and CD16, CD44V6 and CD45, CD44V6 and CD9, CD44V6 and Muc1, CD44V6 and Lewis-Y, CD44V6 and IL1-RAP, CD44V6 and FR-beta, CD44V6 and ROR1, CD47 and CD64, CD47 and CD96, CD47 and CD97, CD47 and CD99, CD47 and CD16, CD47 and CD45, CD47 and CD9, CD47 and Mud, CD47 and Lewis-Y, CD47 and IL1-RAP, CD47 and FR-beta, CD47 and ROR1, CD64 and CD96, CD64 and CD97, CD64 and CD99, CD64 and CD16, CD64 and CD45. CD64 and CD9, CD64 and Muc1, CD64 and Lewis-Y, CD64 and IL1-RAP, CD64 and FR-beta, CD64 and ROR1, CD96 and CD97, CD96 and CD99, CD96 and CD16, CD96 and CD45, CD96 and CD9, CD96 and Muc1, CD96 and Lewis-Y, CD96 and IL1-RAP, CD96 and FR-beta, CD96 and ROR1, CD97 and CD99, CD97 and CD16, CD97 and CD45, CD97 and CD9, CD97 and Muc1, CD97 and Lewis-Y, CD97 and IL1-RAP, CD97 and FR-beta, CD97 and ROR1, CD99 and CD16, CD99 and CD45. CD99 and CD9, CD99 and Muc1, CD99 and Lewis-Y. CD99 and IL1-RAP, CD99 and FR-beta, CD99 and ROR1, CD16 and CD45, CD16 and CD9, CD16 and Muc1, CD16 and Lewis-Y, CD16 and IL1-RAP, CD16 and FR-beta, CD16 and ROR1, CD45 and CD9, CD45 and Muc1, CD45 and Lewis-Y, CD45 and IL1-RAP, CD45 and FR-beta, CD45 and ROR1, CD9 and Muc1, CD9 and Lewis-Y, CD9 and IL1-RAP, CD9 and FR-beta, CD9 and ROR1, MUC1 and Lewis-Y, MUC1 and IL1-RAP. MUC1 and FR-beta, MUC1 and ROR1, Lewis-Y and IL1-RAP, Lewis-Y and FR-beta, Lewis-Y and ROR1, IL1-RAP and FR-beta, IL1-RAP and ROR1, FR-beta and ROR1, B7-H3 and HER2, B7-H3 and CD44v6, B7-H3 and CEA, B7-H3 and CD133, B7-H3 and c-Met, B7-H3 and EGFRvIII, B7-H3 and EPCAM, B7-H3 and EPHA2. B7-H3 and FR-alpha, B7-H3 and GD2, B7-H3 and GPC3, B7-H3 and IL-13R-alpha2, B7-1H3 and IL-11R-alpha, B7-H3 and L1-CAM, B7-H3 and Mesothelin, B7-H3 and MUC1. B7-H3 and MUC16, B7-H3 and IL1-RAP, B7-H3 and CD99. B7-H3 and PSCA, B7-H3 and PSMA, B7-H3 and ROR1, B7-H3 and ALK, HER2 and CD44v6, HER2 and CEA, HER2 and CD133, HER2 and c-Met, HER2 and EGFRvIII, HER2 and EPCAM, HER2 and EPHA2, HER2 and FR-alpha, HER2 and GD2, HER2 and GPC3, HER2 and IL-13R-alpha2, HER2 and IL-11R-alpha, HER2 and L1-CAM, HER2 and Mesothelin, HER2 and MUC1, HER2 and MUC16, HER2 and IL1-RAP, HER2 and CD99, HER2 and PSCA, HER2 and PSMA, HER2 and ROR1, HER2 and ALK, CD44v6 and CEA, CD44v6 and CD133, CD44v6 and c-Met, CD44v6 and EGFRvIII, CD44v6 and EPCAM, CD44v6 and EPHA2, CD44v6 and FR-alpha, CD44v6 and GD2, CD44v6 and GPC3, CD44v6 and IL-13R-alpha2, CD44v6 and IL-11R-alpha, CD44v6 and L1-CAM, CD44v6 and Mesothelin, CD44v6 and MUC1, CD44v6 and MUC16, CD44v6 and IL1-RAP, CD44v6 and CD99, CD44v6 and PSCA, CD44v6 and PSMA, CD44v6 and ROR1, CD44v6 and ALK, CEA and CD133, CEA and c-Met, CEA and EGFRvIII, CEA and EPCAM, CEA and EPHA2, CEA and FR-alpha, CEA and GD2, CEA and GPC3, CEA and IL-13R-alpha2, CEA and IL-11R-alpha, CEA and L1-CAM, CEA and Mesothelin, CEA and MUC1, CEA and MUC16, CEA and IL1-RAP, CEA and CD99, CEA and PSCA, CEA and PSMA, CEA and ROR1, CEA and ALK, CD133 and c-Met, CD133 and EGFRvIII, CD133 and EPCAM, CD133 and EPHA2, CD133 and FR-alpha, CD133 and GD2, CD133 and GPC3, CD133 and IL-13R-alpha2, CD133 and IL-11R-alpha, CD133 and L1-CAM, CD133 and Mesothelin, CD133 and MUC1, CD133 and MUC16, CD133 and IL1-RAP, CD133 and CD99, CD133 and PSCA, CD133 and PSMA. CD133 and ROR1, CD133 and ALK, c-Met and EGFRvIII, c-Met and EPCAM, c-Met and EPHA2, c-Met and FR-alpha, c-Met and GD2, c-Met and GPC3, c-Met and IL-13R-alpha2, c-Met and IL-11R-alpha, c-Met and L1-CAM, c-Met and Mesothelin, c-Met and MUC1, c-Met and MUC16, c-Met and IL1-RAP, c-Met and CD99, c-Met and PSCA, c-Met and PSMA, c-Met and ROR1, c-Met and ALK, EGFRvIII and EPCAM, EGFRvIII and EPHA2, EGFRvIII and FR-alpha, EGFRvIII and GD2, EGFRvIII and GPC3, EGFRvIII and IL-13R-alpha2, EGFRvIII and IL-11R-alpha, EGFRvIII and L1-CAM, EGFRvIII and Mesothelin, EGFRvIII and MUC1, EGFRvIII and MUC16, EGFRvIII and IL1-RAP, EGFRvIII and CD99, EGFRvIII and PSCA, EGFRvIII and PSMA, EGFRvIII and ROR1, EGFRvIII and ALK, EPCAM and EPHA2, EPCAM and FR-alpha, EPCAM and GD2, EPCAM and GPC3, EPCAM and IL-13R-alpha2, EPCAM and IL-11R-alpha, EPCAM and L1-CAM, EPCAM and Mesothelin, EPCAM and MUC1, EPCAM and MUC16, EPCAM and IL1-RAP, EPCAM and CD99, EPCAM and PSCA, EPCAM and PSMA, EPCAM and ROR1, EPCAM and ALK, EPHA2 and FR-alpha, EPHA2 and GD2, EPHA2 and GPC3, EPHA2 and IL-13R-alpha2, EPHA2 and IL-11R-alpha, EPHA2 and L1-CAM, EPHA2 and Mesothelin, EPHA2 and MUC1, EPHA2 and MUC16, EPHA2 and IL1-RAP, EPHA2 and CD99, EPHA2 and PSCA, EPHA2 and PSMA, EPHA2 and ROR1, EPHA2 and ALK, FR-alpha and GD2, FR-alpha and GPC3, FR-alpha and IL-13R-alpha2, FR-alpha and IL-11R-alpha, FR-alpha and L1-CAM, FR-alpha and Mesothelin, FR-alpha and MUC1, FR-alpha and MUC16, FR-alpha and IL1-RAP, FR-alpha and CD99, FR-alpha and PSCA, FR-alpha and PSMA, FR-alpha and ROR1, FR-alpha and ALK, GD2 and GPC3, GD2 and IL-13R-alpha2, GD2 and IL-11R-alpha, GD2 and L1-CAM, GD2 and Mesothelin, GD2 and MUC1, GD2 and MUC16, GD2 and IL1-RAP, GD2 and CD99, GD2 and PSCA, GD2 and PSMA, GD2 and ROR1, GD2 and ALK, GPC3 and IL-13R-alpha2, GPC3 and IL-11R-alpha, GPC3 and L1-CAM, GPC3 and Mesothelin, GPC3 and MUC1, GPC3 and MUC16, GPC3 and IL1-RAP, GPC3 and CD99, GPC3 and PSCA, GPC3 and PSMA, GPC3 and ROR1, GPC3 and ALK, IL-13R-alpha2 and IL-11R-alpha, IL-13R-alpha2 and L1-CAM, IL-13R-alpha2 and Mesothelin, IL-13R-alpha2 and MUC1, IL-13R-alpha2 and MUC16, IL-13R-alpha2 and IL1-RAP, IL-13R-alpha2 and CD99, IL-13R-alpha2 and PSCA, IL-13R-alpha2 and PSMA, IL-13R-alpha2 and ROR1, IL-13R-alpha2 and ALK, IL-11R-alpha and L1-CAM, IL-11R-alpha and Mesothelin, IL-11R-alpha and MUC1, IL-11R-alpha and MUC16, IL-11R-alpha and IL1-RAP, IL-11R-alpha and CD99, IL-11R-alpha and PSCA, IL-11R-alpha and PSMA, IL-11R-alpha and ROR1, IL-11R-alpha and ALK. L1-CAM and Mesothelin. L1-CAM and MUC1, L1-CAM and MUC16, L1-CAM and IL1-RAP. L1-CAM and CD99. L1-CAM and PSCA, L1-CAM and PSMA, L1-CAM and ROR1, L1-CAM and ALK, Mesothelin and MUC1, Mesothelin and MUC16, Mesothelin and IL1-RAP, Mesothelin and CD99, Mesothelin and PSCA, Mesothelin and PSMA, Mesothelin and ROR1, Mesothelin and ALK, MUC1 and MUC16, MUC1 and IL1-RAP, MUC1 and CD99. MUC1 and PSCA. MUC1 and PSMA, MUC1 and ROR1, MUC1 and ALK, MUC16 and IL1-RAP, MUC16 and CD99, MUC16 and PSCA, MUC16 and PSMA, MUC16 and ROR1, MUC16 and ALK, IL1-RAP and CD99, IL1-RAP and PSCA, IL1-RAP and PSMA, IL1-RAP and ROR1, IL1-RAP and ALK, CD99 and PSCA, CD99 and PSMA, CD99 and ROR1, CD99 and ALK. PSCA and PSMA, PSCA and ROR1, PSCA and ALK, PSMA and ROR1, PSMA and ALK, ROR1 and ALK. In any of the preceding antigen combinations, either the first CAR or the second CAR (e.g. the first co-stimulatory and ITAM-containing CAR and the second CD3ε-containing antigen recognizing CAR) can be specific for either of the antigens in the combination. In a non-limiting example, for the CD20 and CD22 antigen combination, the first CAR (co-stimulatory and ITAM-containing CAR) can be specific for CD20 and the second CAR (CD3ε-containing antigen recognizing CAR) can be specific for CD22, or the first CAR (co-stimulatory and ITAM-containing CAR) can be specific for CD22 and the second CAR (CD3ε-containing antigen recognizing CAR) can be specific for CD20.

In addition, the expression of two CARs provides the T cells increased specificity by limiting the off-target toxicity of the cells, such that a signal is only provided to the T cells to kill when the cells contact both antigens expressed on a tumor, as well as enhanced in vivo proliferation and persistence. Thus, normal cells that express only one antigen may not be targeted by the T cells of the disclosure.

Genetic reprogramming of immune cells, such as NK cells and T cells, for adoptive cancer immunotherapy has clinically relevant applications and benefits such as 1) increased ability to recognize tumor cells expressing low levels of antigen 2) increased cell persistence and proliferation. Accordingly, the present disclosure also provides methods for treating immune-related disorders, such as cancer, comprising adoptive cell immunotherapy with any of the engineered immune cells provided herein.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01% Most preferred is a composition in which no amount of the specified component can be detected with standard analytical methods.

As used herein in the specification, “a” or “an” may mean one or more. As used herein in the claim(s), when used in conjunction with the word “comprising,” the words “a” or “an” may mean one or more than one.

As used herein, the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “about” is used to indicate that a value includes the inherent variation of error for the device, the method being employed to determine the value, or the variation that exists among the study subjects.

As used herein, the term “portion” when used in reference to a polypeptide or a peptide refers to a fragment of the polypeptide or peptide. In some embodiments, a “portion” of a polypeptide or peptide retains at least one function and/or activity of the full-length polypeptide or peptide from which it was derived. For example, in some embodiments, if a full-length polypeptide binds a given ligand, a portion of that full-length polypeptide also binds to the same ligand.

The terms “protein” and “polypeptide” are used interchangeably herein.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced into a cell population or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature. The term “exogenous” is used interchangeably with the term “heterologous”.

By “expression construct” or “expression cassette” is used to mean a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide, or the protein expressed by said polynucleotide, to be delivered to a host cell, either m vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

An “origin of replication” (“ori”) or “replication origin” is a DNA sequence, that when present in a plasmid in a cell is capable of maintaining linked sequences in the plasmid and/or a site at or near where DNA synthesis initiates. As an example, an ori for EBV (Ebstein-Barr virus) includes FR sequences (20 imperfect copies of a 30 bp repeat), and preferably DS sequences; however, other sites in EBV bind EBNA-1, e.g., Rep* sequences can substitute for DS as an origin of replication (Kirshmaier and Sugden, 1998). Thus, a replication origin of EBV includes FR, DS or Rep* sequences or any functionally equivalent sequences through nucleic acid modifications or synthetic combination derived therefrom. For example, methods of the present disclosure may also use genetically engineered replication origin of EBV, such as by insertion or mutation of individual elements.

A “gene,” “polynucleotide,” “coding region,” “sequence,” “segment,” “fragment,” or “transgene” that “encodes” a particular protein, is a section of a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located Y to the gene sequence.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein to refer to a nucleotide region comprising a DNA regulatory sequence, wherein the regulatory sequence is derived from a gene that is capable of binding to a RNA polymerase and allowing for the initiation of transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g, a nucleic acid molecule to be transcribed, a promoter, and an functional effector element) are connected in such a way as to permit transcription of the nucleic acid molecule. “Operably linked” with reference to peptide and/or polypeptide molecules means that two or more peptide and/or polypeptide molecules are connected in such a way as to yield a single polypeptide chain. i.e., a fusion polypeptide, having at least one property of each peptide and/or polypeptide component of the fusion. The fusion polypeptide is preferably chimeric, i.e., composed of molecules that are not found in a single polypeptide in nature.

The term “homology” refers to the percent of identity between the nucleic acid residues of two polynucleotides or the amino acid residues of two polypeptides. The correspondence between one sequence and another can be determined by techniques known in the art. For example, homology can be determined by a direct comparison of the sequence information between two polypeptides by aligning the sequence information and using readily available computer programs. Alternatively, homology can be determined by hybridization of polynucleotides under conditions that promote the formation of stable duplexes between homologous regions, followed by digestion with single strand-specific nuclease(s), and size determination of the digested fragments. Two polynucleotide (e.g., DNA), or two polypeptide, sequences are “substantially homologous” to each other when at least about 80%, at least about 90%, and most preferably at least about 95% of the nucleotides, or amino acids, respectively match over a defined length of the molecules, as determined using the methods above.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.)

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPScs, “iPSCs” or “iPS cells”.

An “embryonic stem (ES) cell” is an undifferentiated pluripotent cell which is obtained from an embryo in an early stage, such as the inner cell mass at the blastocyst stage, or produced by artificial means (e.g., nuclear transfer) and can give rise to any differentiated cell type in an embryo or an adult, including germ cells (e.g., sperm and eggs).

“Induced pluripotent stem cells” (iPScs, iPSCs or iPS cells) are cells generated by reprogramming a somatic cell by expressing or inducing expression of a combination of factors (herein referred to as reprogramming factors). iPS cells can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, at least four reprogramming factors, at least five reprogramming factors, at least six reprogramming factors, or at least seven reprogramming factors to reprogram a somatic cell to a pluripotent stem cell.

“Hematopoietic progenitor cells” or “hematopoietic precursor cells” refers to cells which are committed to a hematopoietic lineage but are capable of further hematopoietic differentiation and include hematopoietic stem cells, multipotential hematopoietic stem cells, common myeloid progenitors, megakaryocyte progenitors, erythrocyte progenitors, and lymphoid progenitors.

Hematopoietic stem cells (HSCs) are multipotent stem cells that give rise to all the blood cell types including myeloid (monocytes and macrophages, granulocytes (neutrophils, basophils, eosinophils, and mast cells), erythrocytes, megakaryocytes/platelets, dendritic cells), and lymphoid lineages (T-cells, B cells, NK cells) (see e.g., Doulatov et al., 2012; Notta et al, 2015).

A “multilymphoid progenitor” (MLP) is defined to describe any progenitor that gives rise to all lymphoid lineages (B, T, and NK cells), but that may or may not have other (myeloid) potentials (Doulatov et al., 2010) and is CD45R⁺/ACD10⁺/CD7⁺. Any B, T, and NK progenitor can be referred to as an MLP. A “common myeloid progenitor” (CMP) refers to CD45RA⁺/CD135⁺/CD10⁺/CD7⁺ cells that can give rise to granulocytes, monocytes, megakaryocytes and erythrocytes.

“Pluripotent stem cell” refers to a stem cell that has the potential to differentiate into all cells constituting one or more tissues or organs, or preferably, any of the three germ layers: endoderm (interior stomach lining, gastrointestinal tract, the lungs), mesoderm (muscle, bone, blood, urogenital), or ectoderm (epidermal tissues and nervous system).

As used herein, the term “somatic cell” refers to any cell other than germ cells, such as an egg, a sperm, or the like, which does not directly transfer its DNA to the next generation Typically, somatic cells have limited or no pluripotency. Somatic cells used herein may be naturally-occurring or genetically modified.

“Programming” is a process that alters the type of progeny a cell can produce. For example, a cell has been programmed when it has been altered so that it can form progeny of at least one new cell type, either in culture or in vivo, as compared to what it would have been able to form under the same conditions without programming. This means that after sufficient proliferation, a measurable proportion of progeny having phenotypic characteristics of the new cell type are observed, if essentially no such progeny could form before programming; alternatively, the proportion having characteristics of the new cell type is measurably more than before programming. This process includes differentiation, dedifferentiation and transdifferentiation.

“Differentiation” is the process by which a less specialized cell becomes a more specialized cell type. “Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type. Typically, transdifferentiation by programming occurs without the cells passing through an intermediate pluripotency stage—i.e., the cells are programmed directly from one differentiated cell type to another differentiated cell type Under certain conditions, the proportion of progeny with characteristics of the new cell type may be at least about 1%, 5%, 25% or more in order of increasing preference.

As used herein, the term “subject” or “subject in need thereof refers to a mammal, preferably a human being, male or female at any age that is in need of a therapeutic intervention, a cell transplantation or a tissue transplantation. Typically, the subject is in need of therapeutic intervention, cell or tissue transplantation (also referred to herein as recipient) due to a disorder or a pathological or undesired condition, state, or syndrome, or a physical, morphological or physiological abnormality which is amenable to treatment via therapeutic intervention, cell or tissue transplantation.

As used herein, a “disruption” or “alteration” in reference to a gene refers to a homologous recombination event with a nucleic acid molecule (e.g, an endogenous gene sequence) which results in elimination or reduction of expression of one or more gene products encoded by the subject gene in a cell, compared to the level of expression of the gene product in the absence of the disruption Exemplary gene products include mRNA and protein products encoded by the subject gene. Alteration in some cases is transient or reversible and in other cases is permanent. Alteration in some cases is of a functional or full-length protein or mRNA, despite the fact that a truncated or nonfunctional product may be produced. In some embodiments herein, gene activity or function, as opposed to expression, is disrupted. Gene alteration is generally induced by artificial methods, i.e., by addition or introduction of a compound, molecule, complex, or composition, and/or by alteration of nucleic acid of or associated with the gene, such as at the DNA level. Exemplary methods for gene alteration include gene silencing, knockdown, knockout, and/or gene alteration techniques, such as gene editing. Examples of gene editing methods include CRISPR/Cas systems, meganuclease systems, Zinc Finger Protein (ZFP) and Zinc Finger Nuclease (ZFN) systems and/or transcription activator-like protein (TAL), transcription activator-like effector protein (TALE) or TALE nuclease protein (TALEN) systems. Examples of gene alteration also include antisense technology, such as RNAi, siRNA, shRNA, and/or ribozymes, which generally result in transient reduction of expression, as well as gene editing techniques which result in targeted gene inactivation or alteration, e.g., by induction of breaks and/or homologous recombination. Examples include insertions, mutations, and deletions. The alterations typically result in the repression and/or complete absence of expression of a normal or “wild-type” product encoded by the gene Exemplary of such gene alterations are insertions, frameshift and missense mutations, deletions, substitutions, knock-in, and knock-out of the gene or part of the gene, including deletions of the entire gene. Such alterations can occur in the coding region, e.g., in one or more exons, resulting in the inability to produce a full-length product, functional product, or any product, such as by insertion of a stop codon Such alterations may also occur by alterations in the promoter or enhancer or other region affecting activation of transcription, so as to prevent transcription of the gene. Gene alterations include gene targeting, including targeted gene inactivation by homologous recombination.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a NK cell, B cell, or a T cell, or innate immune cell to a stimulus. In one embodiment, the response is specific for a particular antigen (an “antigen-specific response”).

As used herein, the term “antigen” is a molecule capable of being bound by an antibody, T-cell receptor. Chimeric Antigen Receptor and or engineered immune receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

An “epitope” is the site on an antigen recognized by an antibody as determined by the specificity of the amino acid sequence. Two antibodies are said to bind to the same epitope if each competitively inhibits (blocks) binding of the other to the antigen as measured in a competitive binding assay. Alternatively, two antibodies bind to the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

An “autoimmune disease” refers to a disease in which the immune system produces an immune response (for example, a B-cell or a T-cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces.

The term “Graft-Versus-Host Disease (GVHD)” refers to a common and serious complication of bone marrow or other tissue transplantation wherein there is a reaction of donated immunologically competent lymphocytes against a transplant recipient's own tissue. GVHD is a possible complication of any transplant that uses or contains stem cells from either a related or an unrelated donor In some embodiments, the GVHD is chronic GVHD (cGVHD).

A “parameter of an immune response” is any particular measurable aspect of an immune response, including, but not limited to, cytokine secretion (IFN-γ, etc.), chemokine secretion, altered migration or cell accumulation, immunoglobin production, dendritic cell maturation, regulatory activity, number of immune cells and proliferation of any cell of the immune system.

Another parameter of an immune response is structural damage or functional deterioration of any organ resulting from immunological attack One of skill in the art can readily determine an increase in any one of these parameters, using known laboratory assays. In one specific non-limiting example, to assess cell proliferation, incorporation of ³H-thymidine can be assessed. A “substantial” increase in a parameter of the immune response is a significant increase in this parameter as compared to a control. Specific, non-limiting examples of a substantial increase are at least about a 50% increase, at least about a 75% increase, at least about a 90% increase, at least about a 100% increase, at least about a 200% increase, at least about a 300% increase, and at least about a 500% increase. Similarly, an inhibition or decrease in a parameter of the immune response is a significant decrease in this parameter as compared to a control. Specific, non-limiting examples of a substantial decrease are at least about a 50% decrease, at least about a 75% decrease, at least about a 90% decrease, at least about a 100% decrease, at least about a 200% decrease, at least about a 300% decrease, and at least about a 500% decrease. A statistical test, such as a non-parametric ANOVA, or a T-test, can be used to compare differences in the magnitude of the response induced by one agent as compared to the percent of samples that respond using a second agent. In some examples, p≤0.05 is significant, and indicates that the chance that an increase or decrease in any observed parameter is due to random variation is less than 5%. One of skill in the art can readily identify other statistical assays of use.

“Treating” or treatment of a disease or condition refers to executing a protocol or treatment plan, which may include administering one or more drugs to a patient, in an effort to alleviate signs or symptoms of the disease or the recurrence of the disease. Desirable effects of treatment include decreasing the rate of disease progression, ameliorating or palliating the disease state, and remission, increased survival, improved quality of life or improved prognosis.

Alleviation or prevention can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols or treatment plans that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis or recurrence. Treatment of cancer may also refer to prolonging survival of a subject with cancer.

“Antigen recognition moiety” or “antigen recognition domain” refers to a molecule or portion of a molecule that specifically binds to an antigen. In one embodiment, the antigen recognition moiety is an antibody, antibody like molecule or fragment thereof and the antigen is a tumor antigen.

“Antibody” as used herein refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CHL CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

“Antibody like molecules” may be for example proteins that are members of the Ig-superfamily which are able to selectively bind a partner.

The terms “fragment of an antibody,” “antibody fragment,”, “functional fragment of an antibody,” and “antigen-binding portion” are used interchangeably herein to mean one or more fragments or portions of an antibody that retain the ability to specifically bind to an antigen (see, generally, Holliger et al. (2005) Nat. Biotech. 23(9) 1126-29). The antibody fragment desirably comprises, for example, one or more CDRs, the variable region (or portions thereof), the constant region (or portions thereof), or combinations thereof.

Examples of antibody fragments include, but are not limited to, (i) a Fab fragment, which is a monovalent fragment consisting of the VL, VH, CL, and CH1 domains; (ii) a F(ab′)2 fragment, which is a bivalent fragment comprising two Fab fragments linked by a disulfide bridge at the stalk region; (iii) a Fv fragment consisting of the VL and VH domains of a single arm of an antibody, (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al. (1988), Science 242: 423-6, Huston et al. (1988) Proc. Natl. Acad. Sci. USA 85: 5879-83; and Osbourn et al. (1998) Nat. Biotechnol. 16: 778-81) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Patent Application Publication 2009/0093024 A1.

A “chimeric antigen receptor” is also known as an artificial cell receptor, a chimeric cell receptor, or a chimeric immunoreceptor. Chimeric antigen receptors (CARs) are engineered receptors, which graft a selected specificity onto an immune effector cell. CARs typically have an extracellular domain (ectodomain), which comprises an antigen-binding domain and a stalk region, a transmembrane domain and an intracellular (endodomain) domain.

A “stalk region”, which encompasses the terms “spacer region” or “hinge domain” or “hinge”, is used to link the antigen-binding domain to the transmembrane domain. As used herein, the term “stalk region” generally means any oligonucleotide or polypeptide that functions to link the transmembrane domain to, either the extracellular domain or, the cytoplasmic domain in the polypeptide chain of a CAR. In embodiments, it is flexible enough to allow the antigen-binding domain to orient in different directions to facilitate antigen recognition.

The term “functional portion,” when used in reference to a CAR, refers to any part or fragment of a CAR described herein, which part or fragment retains the biological activity of the CAR of which it is a part (the parent CAR). In reference to a nucleic acid sequence encoding the parent CAR, a nucleic acid sequence encoding a functional portion of the CAR can encode a protein comprising, for example, about 10%, 25%, 30%, 50%, 68%, 80%, 90%, 95%, or more, of the parent CAR.

The term “functional variant,” as used herein, refers to a polypeptide, or a protein having substantial or significant sequence identity or similarity to the reference polypeptide, and retains the biological activity of the reference polypeptide of which it is a variant. Functional variants encompass, for example, those variants of the CAR described herein (the parent CAR) that retain the ability to recognize target cells to a similar extent, the same extent, or to a higher extent, as the parent CAR. In reference to a nucleic acid sequence encoding the parent CAR, a nucleic acid sequence encoding a functional variant of the CAR can be for example, about 10% identical, about 25% identical, about 30% identical, about 50% identical, about 65% identical, about 70% identical, about 75% identical, about 80% identical, about 85% identical, about 90% identical, about 95% identical, or about 99% identical to the nucleic acid sequence encoding the parent CAR.

The phrases “pharmaceutical or pharmacologically acceptable” refers to molecular entities and compositions that do not produce an adverse, allergic, or other untoward reaction when administered to an animal, such as a human, as appropriate. For animal (e.g., human) administration, it will be understood that preparations should meet sterility, pyrogenicity, general safety, and purity standards as required, e.g., by the FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “T cell” refers to T lymphocytes, and includes, but is not limited to, γ/δ T cells, α/β T cells. NK T cells, CD4⁺ T cells and CD8⁺ T cells CD4⁺ T cells include THO, Tₕ1 and TH2 cells, as well as regulatory T cells (T_{reg}). There are at least three types of regulatory T cells: CD4⁺ CD25⁺ T_{reg}, CD25 T_{H}3 T_{reg}, and CD25 T_{R} 1 T_{reg}. “Cytotoxic T cell” refers to a T cell that can kill another cell. The majority of cytotoxic T cells are CD8⁺ MHC class I-restricted T cells, however some cytotoxic T cells are CD4⁺. In preferred embodiments, the T cell of the present disclosure is CD4⁺ or CD8⁺.

The activation state of a T cell defines whether the T cell is “resting” (i.e., in the Gₒ phase of the cell cycle) or “activated” to proliferate after an appropriate stimulus such as the recognition of its specific antigen, or by stimulation with OKT3 antibody, PHA or PMA, etc. The “phenotype” of the T cell (e.g., naive, central memory, effector memory, lytic effectors, help effectors (TH1 and TH2 cells), and regulatory effectors), describes the function the cell exerts when activated. A healthy donor has T cells of each of these phenotypes, and which are predominately in the resting state. A naive T cell will proliferate upon activation, and then differentiate into a memory T cell or an effector T cell. It can then assume the resting state again, until it gets activated the next time, to exert its new function and may change its phenotype again. An effector T cell will divide upon activation and antigen-specific effector function.

“Natural killer T cells” (NKT cells) not to be confused with natural killer cells of the innate immune system) bridge the adaptive immune system with the innate immune system. Unlike conventional T cells that recognize peptide antigens presented by major histocompatibility complex (WIC) molecules, NKT cells recognize glycolipid antigen presented by a molecule called CD1d Once activated, these cells can perform functions ascribed to both Th and Tc cells (i.e., cytokine production and release of cytolytic/cell killing molecules). They are also able to recognize and eliminate some tumor cells and cells infected with herpes viruses.

“Natural killer cells” (“NK cells”) are a type of cytotoxic lymphocyte of the innate immune system. In some instances, NK cells provide a first line defense against viral infections and/or tumor formation. NK cells can detect MHC presented on infected or cancerous cells, triggering cytokine release, and subsequently induce lysis and apoptosis. NK cells can further detect stressed cells in the absence of antibodies and/or MHC, thereby allowing a rapid immune response.

“Tumor antigen” as used herein refers to any antigenic substance produced, expressed or overexpressed in tumor cells. It may, for example, trigger an immune response in the host.

Alternatively, for purposes of this disclosure, tumor antigens may be proteins that are expressed by both healthy and tumor cells but because they identify a certain tumor type, are a suitable therapeutic target. In one embodiment, the tumor antigen is CD22. In one embodiment, the tumor antigen is CD19.

The term “antigen presenting cells (APCs)” refers to a class of cells capable of presenting one or more antigens in the form of peptide-MHC complex recognizable by specific effector cells of the immune system, and thereby inducing an effective cellular immune response against the antigen or antigens being presented. APCs can be intact whole cells such as macrophages, B cells, endothelial cells, activated T cells, and dendritic cells; or other molecules, naturally occurring or synthetic, such as purified MHC Class I molecules complexed to 2-microglobulin.

The term “culturing” refers to the in vitro maintenance, differentiation, and/or propagation of cells in suitable media. By “enriched” is meant a composition comprising cells present in a greater percentage of total cells than is found in the tissues where they are present in an organism.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

### II. IMMUNE CELLS

Certain embodiments of the present disclosure concern immune cells which express a chimeric antigen receptor (CAR). The immune cells may be T cells (e.g, regulatory T cells, CD4⁺ T cells, CD8⁺ T cells, or gamma-delta T cells), NK cells, invariant NK cells, NKT cells, stem cells (e.g., mesenchymal stem cells (MSCs) or induced pluripotent stem (iPSC) cells). In some embodiments, the cells are monocytes or granulocytes, e.g, myeloid cells, macrophages, neutrophils, dendritic cells, mast cells, eosinophils, and/or basophils. Also provided herein are methods of producing and engineering the immune cells and methods of using and administering the cells for adoptive cell therapy, in which case the cells may be autologous or allogeneic. Thus, the immune cells may be used as immunotherapy, such as to target cancer cells.

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the noni-living subjects are organ donors. The isolated immune cells may be used directly, or they can be stored for a period of time, such as by freezing. In some embodiments, the immune cells are isolated from blood, such as peripheral blood or cord blood. In some embodiments, immune cells isolated from cord blood have enhanced immunomodulation capacity, such as measured by CD4-positive or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood. The population of immune cells can be derived from induced pluripotent stem cells (iPSCs) and/or any other stem cell known in the art. In some aspects, the iPSCS and/or stem cells used to derive the population of immune cells can be obtained from a subject in need of therapy or suffering from a disease associate with reduced immune cell activity, thus these IPSCs and/or stem cells will be autologous to the subject in need of therapy. Alternatively, the iPSCs and/or stem cells can be obtained from a donor and therefore be allogeneic to the subject in need of therapy.

When the population of immune cells is obtained from a donor distinct from the subject, the donor is preferably allogeneic, provided the cells obtained are subject-compatible in that they can be introduced into the subject. Allogeneic donor cells are may or may not be human leukocyte antigen (HLA)-compatible. To be rendered subject-compatible, allogeneic cells can be treated to reduce immunogenicity.

### 1. T Cells

T-cells play a major role in cell-mediated-immunity (no antibody Involvement). Its T-cell receptors (TCR) differentiate themselves from other lymphocyte types. The thymus, a specialized organ of the immune system, is primarily responsible for the T cell's maturation. There are six types of T-cells, namely. Helper T-cells (e.g CD4+ cells), Cytotoxic T-cells (also known as TC, cytotoxic T lymphocyte, CTL, T-killer cell, cytolytic T cell, CD78+ T-cells or killer T cell), Memory T-cells ((i) stem memory TSCM cells, like naive cells, are CD45RO−, CCR7+, CD45RA+, CD62L+(L-selectin), CD27+, CD28+ and IL-7Ra+, but they also express large amounts of CD95, IL-2R, CXCR3, and LFA-1, and show numerous functional attributes distinctive of memory cells), (ii) central memory TCM cells express L-selectin and the CCR7, they secrete IL-2, but not IFNg or IL4, and (iii) effector memory TEM cells, however, do not express L-selectin or CCR7 but produce effector cytokines like IFNg and IL-4), Regulatory T-cells (Tregs, suppressor T cells, or CD4+CD25+ regulatory T cells), Natural Killer T-cells (NKT) and Gamma Delta T-cells.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U S. Patent Publication No. 2013/0287748, which is herein incorporated by references in its entirety.

### 2. Genetically Engineered Antigen Receptors

The immune cells of the disclosure (e.g., autologous or allogeneic T cells (e.g., regulatory T cells. CD4⁺ T cells, CD8⁺ T cells, or gamma-delta T cells), NK cells, invariant NK cells, NKT cells, stem cells (e.g., MSCs or iPS cells) can be genetically engineered to express antigen receptors such as engineered CARs and/or TCRs. For example, the host cells (e.g, autologous or allogeneic T cells) are modified to express a CAR having antigenic specificity for a cancer antigen. In particular embodiments, T cells are engineered to express a CAR. The T cells may be further engineered to express a TCR. Multiple CARs and/or TCRs, such as to different antigens, may be added to a single cell type, such as T cells.

Suitable methods of modification are known in the art. See, for instance, Sambrook and Ausubel, supra. For example, the cells may be transduced to express a TCR having antigenic specificity for a cancer antigen using transduction techniques described in Heemskerk et al., 2008 and Johnson et al., 2009.

In some embodiments, the cells comprise one or more nucleic acids introduced via genetic engineering that encode one or more antigen receptors, and genetically engineered products of such nucleic acids. In some embodiments, the nucleic acids are heterologous. In some embodiments, the nucleic acids are not naturally occurring, such as a nucleic acid not found in nature (e.g., chimeric).

In some embodiments, the CAR contains an extracellular antigen-recognition domain that specifically binds to an antigen (e.g., a tumor antigen or a pathogen antigen). In some embodiments, the antigen is a protein expressed on the surface of cells (e.g., cancerous cells).

Exemplary engineered antigen receptors, including CARs and recombinant TCRs, as well as methods for engineering and introducing the receptors into cells, include those described, for example, in PCT Publication Nos. WO 2000/14257, WO 2013126726, WO 2012/129514, WO 2014/031687, WO 2013/166321, WO 2013/071154, and WO 2013/123061, U.S. Patent Application Publication Nos. US 2002/131960, US 2013/287748, and US 2013/0149337; and U.S. Pat. Nos. 6,451,995, 7,446,190, 8,252,592, 8,339,645, 8,398,282, 7,446,179, 6,410,319, 7,070,995, 7,265,209, 7,354,762, 7,446,190, 7,446,191, 8,324,353, and 8,479,118: International Patent Application Publication No.: WO 2014/055668 A1, and European Patent Application Publication No. EP2537416; and/or those described by Sadelain et al., 2013; Davila et al., 2013; Turtle et al, 2012; Wu et al., 2012.

### 3. Chimeric Antigen Receptors

In some aspects, the present disclosure provides a population of genetically modified immune cells (e.g. T cells) engineered to express a first chimeric antigen receptor (CAR) and/or a polynucleotide encoding a CAR, wherein the CAR comprises (a) an antigen recognition domain that specifically binds to a first antigen (e.g. CD22), a transmembrane domain; and an intracellular signaling domain and (b) a second chimeric antigen receptor (CAR) and/or a polynucleotide encoding a CAR, wherein the second CAR comprises (a) an antigen recognition domain that specifically binds to an antigen, wherein the antigen may differ from the antigen to which the first CAR binds (e.g. CD19) or may be the same antigen to which the first CAR binds (e.g. CD22); a transmembrane domain; and a CD3e intracellular signaling domain. In some embodiments, the intracellular domain of the first CAR comprises one or more (e.g., one, two, three, or more) co-stimulatory domains.

In some embodiments, the genetically engineered cells include additional CARs, including activating or stimulatory CARs, co-stimulatory CARs (see, e.g., PCT Publ. No. WO 2014/055668), and/or inhibitory CARs (iCARs, see, e.g., Fedorov et al., 2013). The CARs generally include an extracellular antigen (or ligand) recognition domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone. For example, once an antigen is recognized by the extracellular antigen recognition domain, the intracellular signaling components transmit an activation signal to the T cell that induces the T cell to destroy a targeted tumor cell.

### A. Antigen Recognition Domains

In some embodiments, the antigen recognition domain of the CARs described herein may recognize an epitope comprising the shared space between one or more antigens. In some embodiments, the antigen recognition domain comprises complementary determining regions (CDRs) of a monoclonal antibody, variable regions of a monoclonal antibody, an scFv, a VH, a VHH, a single domain antibody (e.g, a camelid single domain antibody), an antibody mimetic and/or antigen binding fragments thereof. In some embodiments, the specificity of the antigen recognition domain is derived from a protein or peptide (e.g., a ligand in a receptor-ligand pair) that specifically binds to another protein or peptide (e.g, a receptor in a receptor-ligand pair) In some embodiments, the antigen recognition domain comprises an aptamer, a T cell receptor (TCR)-like antibody, or a single chain TCR (scTCR). Almost any moiety that binds a given target (e.g., tumor associated antigen (TAA)) with sufficient affinity can be used as an antigen recognition domain. The arrangement of the antigen recognition domain could be multimeric, such as a diabody or multimers. In some embodiments, the multimers can be formed by cross pairing of the variable portion of the light and heavy chains into a diabody.

In some embodiments, the antigen recognition domain of the CARs described herein comprises an antibody mimetic. The term “antibody mimetic” is intended to describe an organic compound that specifically binds a target sequence and has a structure distinct from a naturally-occurring antibody. Antibody mimetics may comprise a protein, a nucleic acid, or a small molecule. The target sequence to which an antibody mimetic of the disclosure specifically binds may be an antigen. Exemplary antibody mimetics include, but are not limited to, an affibody, an affilin, an affimer, an affitin, an alphabody, an anticalin, an avimer (also known as avidity multimer), a DARPin (Designed Ankyrin Repeat Protein), a Fynomer, a Kunitz domain peptide, a monobody and a centyrin.

In some embodiments, the first CAR provided herein comprise a single chain variable fragments (scFv) derived from monoclonal antibodies specific for tumor associated antigen (e.g., CD22), a hinge domain, a transmembrane domain, and an ITAM-containing intracellular signaling domain (e.g. CD3z). Such molecules result in the transmission of an ITAM-mediated signal in response to recognition by the scFv of its target. In some embodiments, the first CAR further comprises an additional intracellular signaling domain (“costimulatory domain”).

In some embodiments, the second CAR provided herein comprises a single chain variable fragments (scFv) derived from monoclonal antibodies specific for tumor associated antigen (e.g, CD19), a hinge domain, a transmembrane domain, and a CD3ε intracellular signaling domain.

Nucleic acids encoding any of the CARs described herein are also provided. Nucleic acids encoding the CAR may be humanized. In some embodiments, the nucleic acid encoding a CAR provided herein is codon-optimized for expression in human cells. In some embodiments, the disclosure provides a full-length CAR cDNA or coding region.

In some embodiments, the antigen recognition domain of a CAR provided herein comprises a fragment of the VH and VL chains of a single-chain variable fragment (scFv) that specifically bind CD22. Accordingly, the antigen recognition domain of a CAR, provided herein can comprise any scFv known in the art to specifically bind CD22.

In some embodiments, the antigen recognition domain of a CAR provided herein comprises a fragment of the VH and VL chains of a single-chain variable fragment (scFv) that specifically bind CD19 such as those described in U.S. Patent Appl. Publ. Nos. 2020/0246382, PCT Appl. Publ. Nos. WO 2020223445 and WO 2020123691, each of which is incorporated herein by reference in its entirety. Accordingly, the antigen recognition domain of a CAR provided herein can comprise any scFv known in the art to specifically bind CD19.

In some embodiments, the antigen recognition domain of the CAR described herein binds (e.g. specifically binds) to the antigens described in Table 1. The antigen specific CAR, when expressed on the cell surface, redirects the specificity of immune cells (e.g. T cells) to the respective antigen.

TABLE 1
Exemplary Targets of Antigen Recognition Domains

| | | |
|---|---|---|
| Protein Name | UniProt ID | NCBI Accession No. |

B cell malignancies

| | | | |
|---|---|---|---|
| CD19 | B-lymphocyte antigen CD19; Cluster | P15391 | NM_001178098 |
| | of Differentiation 19; B-Lymphocyte | | |
| | Surface Antigen B4; T-Cell Surface | | |
| | Antigen Leu-12; CVID3 | | |
| CD22 | Cluster of Differentiation 22 | P20273 | NM_001185099 |
| CD20 | B-lymphocyte antigen CD20; B- | P11836 | NM_021950 |
| | lymphocyte cell-surface antigen B1; | | NM_152866 |
| | CD20 antigen; CD20 receptor; | | NM_152867 |
| | leukocyte surface antigen Leu-16; | | |
| | membrane-spanning 4-domains, | | |
| | subfamily A, member 1 | | |
| CD138 | syndecan-1; CD138 antigen; heparan | P18827 | NM_001006946 |
| | sulfate proteoglycan fibroblast | | NM_002997 |
| | growth factor receptor; syndecan | | |
| | proteoglycan 1; SDC; CD138; | | |
| | SYND1; syndecan | | |
| BCMA | Tumor necrosis factor receptor | Q02223 | NM_001192 |
| | superfamily member 17 | | |
| | (TNFRSF17); B cell maturation | | |
| | antigen; B-cell maturation factor; B- | | |
| | cell maturation protein; BCM; | | |
| | BCMA; CD269; TNFRSF13A | | |
| CD10 | | | |
| CD5 | T-cell surface glycoprotein CD5; | P06127 | NM_001346456 |
| | CD5 antigen (p56-62); epididymis | | NM_014207 |
| | secretory sperm binding protein; | | |
| | lymphocyte antigen T1/Leu-1; T1; | | |
| | LEU1; CD5 molecule | | |
| CD79a | | | |
| CD79b | | | |

Myeloid Malignancies

| | | | |
|---|---|---|---|
| CD33 | myeloid cell surface antigen CD33; | P20138 | NM_001082618 |
| | CD33 antigen (gp67); CD33 | | NM_001177608 |
| | molecule transcript; gp67; sialic acid- | | NM_001772 |
| | binding Ig-like lectin 3; p67; | | |
| | SIGLEC3; SIGLEC-3 | | |
| CD123 | interleukin-3 receptor subunit alpha; | P26951 | NM_001267713 |
| | CD123 antigen; IL-3 receptor subunit | | NM_002183 |
| | alpha; IL-3R subunit alpha; IL-3R- | | |
| | alpha; IL-3RA; interleukin 3 | | |
| | receptor, alpha (low affinity); IL3R; | | |
| | CD123; IL3RX; IL3RY; IL3RAY; | | |
| | hIL-3Ra | | |
| FLT3 | receptor-type tyrosine-protein kinase | P36888 | NM_004119 |
| | FLT3; CD135 antigen; FL cytokine | | |
| | receptor; fetal liver kinase 2; fms | | |
| | related tyrosine kinase 3; fms-like | | |
| | tyrosine kinase 3; growth factor | | |
| | receptor tyrosine kinase type III; | | |
| | stem cell tyrosine kinase 1; FLK2; | | |
| | STK1; CD135; FLK-2 | | |
| CLEC1A | C-type lectin domain family 1 | Q8NC01 | NM_001297748 |
| | member A; C-type lectin-like | | NM_001297749 |
| | receptor-1; CLEC1; CLEC-1 | | NM_001297750 |
| | | | NM_001297751 |
| | | | NM_016511 |
| CD56 | neural cell adhesion molecule 1; | P13591 | NM_000615 |
| | antigen recognized by monoclonal | | NM_001076682 |
| | antibody 5.1H11; neural cell | | NM_001242607 |
| | adhesion molecule, NCAM; CD56; | | NM_001242608 |
| | NCAM; MSK39 | | NM_001386289 |
| | | | NM_001386290 |
| | | | NM_001386291 |
| | | | NM_001386292 |
| | | | NM_181351 |
| CD34 | hematopoietic progenitor cell antigen | P28906 | NM_001025109 |
| | CD34; CD34 antigen; CD34 molecule | | NM_001773 |
| CD117 | KIT proto-oncogene, receptor | P10721 | NM_000222 |
| | tyrosine kinase; mast/stem cell | | NM_001093772 |
| | growth factor receptor Kit; c-Kit | | NM_001385284 |
| | protooncogene; p145 c-kit; piebald | | NM_001385285 |
| | trait protein; proto-oncogene c-Kit; | | NM_001385286 |
| | proto-oncogene tyrosine-protein | | NM_001385288 |
| | kinase Kit; soluble KIT variant 1; | | NM_001385290 |
| | tyrosine-protein kinase Kit; v-kit | | NM_001385292 |
| | Hardy-Zuckerman 4 feline sarcoma | | |
| | viral oncogene homolog; v-kit | | |
| | Hardy-Zuckerman 4 feline sarcoma | | |
| | viral oncogene-like protein; PBT; | | |
| | SCFR; C-Kit; CD117; MASTC | | |
| CD14 | CD14 molecule; monocyte | P08571 | NM_000591 |
| | differentiation antigen CD14; | | NM_001040021 |
| | myeloid cell-specific leucine-rich | | NM_001174104 |
| | glycoprotein; Cluster of | | NM_001174105 |
| | Differentiation 14 | | |
| CD133 | prominin-1; antigen AC133; | O43490 | NM_001145847 |
| | hProminin; hematopoietic stem cell | | NM_001145848 |
| | antigen; prominin-like protein 1; | | NM_001145849 |
| | RP41; AC133; CD133; MCDR2; | | NM_001145850 |
| | STGD4; CORD12; PROML1; | | NM_001145851 |
| | MSTP061 | | NM_001145852 |
| | | | NM_001371406 |
| | | | NM_001371407 |
| | | | NM NM_006017 |
| | | | _001371408 |
| CD44v6 | CD44 molecule variant 6; CD44 | P16070-6 | NM_001202555 |
| | antigen variant 6; CD44 molecule | | |
| | isoform 6; | | |
| CD47 | leukocyte surface antigen CD47; | Q08722 | NM_001382306 |
| | CD47 antigen (Rh-related antigen, | | NM_001777 |
| | integrin-associated signal | | NM_198793 |
| | transducer); CD47 glycoprotein; Rh- | | |
| | related antigen; antigen identified by | | |
| | monoclonal antibody 1D8; antigenic | | |
| | surface determinant protein OA3; | | |
| | integrin associated protein; integrin- | | |
| | associated signal transducer; CD47 | | |
| | molecule | | |
| CD64 | high affinity immunoglobulin gamma | P12314 | NM_000566 |
| | Fc receptor I; Fc fragment of IgG, | | NM_001378804 |
| | high affinity Ia, receptor (CD64); Fc | | NM_001378805 |
| | fragment of IgG, high affinity Ia, | | NM_001378806 |
| | receptor for (CD64); Fc gamma | | NM_001378807 |
| | receptor Ia; Fc-gamma RI; Fc-gamma | | NM_001378808 |
| | receptor I A1; IgG Fc receptor I; fc- | | NM_001378809 |
| | gamma RIA; fcgammaRIa; Fc | | NM_001378810 |
| | fragment of IgG receptor Ia; CD64; | | NM_001378811 |
| | FCRI; CD64A; IGFR1 | | |
| CD96 | T-cell surface protein tactile; T cell | P40200 | NM_001318889 |
| | activation, increased late expression; | | NM_005816 |
| | cell surface antigen CD96; t cell- | | NM_198196 |
| | activated increased late expression | | |
| | protein; TACTILE; CD96 molecule | | |
| CD97 | adhesion G protein-coupled receptor | P48960 | NM_001025160 |
| | E5; CD97 molecule; leukocyte | | NM_001784 |
| | antigen CD97; seven transmembrane | | NM_078481 |
| | helix receptor; seven-span | | |
| | transmembrane protein; seven- | | |
| | transmembrane, heterodimeric | | |
| | receptor associated with | | |
| | inflammation; adhesion G protein- | | |
| | coupled receptor E5; TM7LN1 | | |
| CD99 | CD99 antigen; E2 antigen; MIC2 | P14209 | NM_001122898 |
| | (monoclonal antibody 12E7); T-cell | | NM_001321367 |
| | surface glycoprotein E2; antigen | | NM_001321368 |
| | identified by monoclonal 12E7, Y | | NM_001321369 |
| | homolog; antigen identified by | | NM_001321370 |
| | monoclonal antibodies 12E7, F21 | | NM_002414 |
| | and O13; cell surface antigen 12E7; | | |
| | cell surface antigen HBA-71; cell | | |
| | surface antigen O13; surface antigen | | |
| | MIC2; CD99 molecule (Xg blood | | |
| | group); HBA71; MIC2X; MIC2Y; | | |
| | MSK5X | | |
| CD16 | low affinity immunoglobulin gamma | P08637 | NM_000569 |
| | Fc region receptor III-A; CD16a | | NM_001127592 |
| | antigen; Fc fragment of IgG, low | | NM_001127593 |
| | affinity III, receptor for (CD16); Fc | | NM_001127595 |
| | fragment of IgG, low affinity IIIa, | | NM_001127596 |
| | receptor (CD16a); Fc gamma | | NM_001329120 |
| | receptor III-A; Fc-gamma RIII-alpha; | | NM_001329122 |
| | Fc-gamma receptor III-2 (CD 16); | | NM_001386450 |
| | Fc-gamma receptor IIIb (CD16); | | |
| | FcgammaRIIIA; igG Fc receptor III- | | |
| | 2; immunoglobulin G Fc receptor III; | | |
| | low affinity immunoglobulin gamma | | |
| | receptor III-a Fc fragment; | | |
| | neutrophil-specific antigen NA; | | |
| | CD16; FCG3; CD16A; FCGR3; | | |
| | IGFR3; IMD20; FCR-10; FCRIII; | | |
| | FCGRIII; FCRIIIA; Fc fragment of | | |
| | IgG receptor IIIa | | |
| CD45 | receptor-type tyrosine-protein | P08575 | NM_001267798 |
| | phosphatase C; CD45 antigen; T200 | | NM_002838 |
| | glycoprotein; T200 leukocyte | | NM_080921 |
| | common antigen; protein tyrosine | | |
| | phosphatase, receptor type, c | | |
| | polypeptide; protein tyrosine | | |
| | phosphatase receptor type C; PTPRC; | | |
| | LCA; LY5; B220; CD45; L-CA; | | |
| | T200; CD45R; GP180 | | |
| CD9 | CD9 antigen; 5H9 antigen; BA-2/p24 | P21926 | NM_001330312 |
| | antigen; CD9 antigen (p24); antigen | | NM_001769 |
| | CD9; cell growth-inhibiting gene 2 | | |
| | protein; leukocyte antigen MIC3; | | |
| | motility related protein-1; | | |
| | tetraspanin-29; MIC3; MRP-1; | | |
| | BTCC-1; DRAP-27; TSPAN29; | | |
| | TSPAN-29; CD9 molecule | | |
| Muc1 | mucin-1; H23 antigen; breast | P15941 | NM_001018016 |
| | carcinoma-associated antigen DF3; | | NM_001018017 |
| | cancer antigen 15-3; carcinoma- | | NM_001044390 |
| | associated mucin; episialin; krebs | | NM_001044391 |
| | von den Lungen-6; mucin 1, | | NM_001044392 |
| | transmembrane; peanut-reactive | | NM_001044393 |
| | urinary mucin; polymorphic | | NM_001204285 |
| | epithelial mucin; tumor associated | | NM_001204286 |
| | epithelial mucin; tumor-associated | | NM_001204287 |
| | epithelial membrane antigen; EMA; | | NM_001204288 |
| | MCD; PEM; PUM; KL-6; MAM6; | | NM_001204289 |
| | MCKD; PEMT; CD227; H23AG; | | NM_001204290 |
| | MCKD1; MUC-1; ADMCKD; | | NM_001204291 |
| | ADTKD2; ADMCKD1; CA 15-3; | | NM_001204292 |
| | MUC-1/X; MUC1/ZD; MUC-1/SEC | | NM_001204293 |
| | | | NM_001204294 |
| | | | NM_001204295 |
| | | | NM_001204296 |
| | | | NM_001204297 |
| | | | NM_001371720 |
| | | | NM_002456 |
| Lewis-Y | | | |
| IL1RAP | interleukin-1 receptor accessory | Q9NPH3 | NM_001167928 |
| | protein; IL-1 receptor accessory | | NM_001167929 |
| | protein; interleukin-1 receptor 3; | | NM_001167930 |
| | interleukin-1 receptor accessory | | NM_001167931 |
| | protein beta; IL1R3; C3orf13; IL- | | NM_001364879 |
| | 1RAcP; IL1RAP | | NM_001364880 |
| | | | NM_001364881 |
| | | | NM_002182 |
| | | | NM_134470 |
| FR-beta | folate receptor beta; folate receptor 2 | P14207 | NM_000803 |
| | (fetal); folate receptor alpha; folate | | NM_001113534 |
| | receptor, fetal/placental; folate- | | NM_001113535 |
| | binding protein, fetal/placental; | | NM_001113536 |
| | placental folate-binding protein; | | |
| | FBP; FOLR1; FR-P3; FRbeta; FR- | | |
| | BETA; BETA-HFR; FBP/PL-1; | | |
| | FOLR2 | | |

T cell malignancies

| | | | |
|---|---|---|---|
| CD5 | T-cell surface glycoprotein CD5; | P06127 | NM_001346456 |
| | CD5 antigen (p56-62); epididymis | | NM_014207 |
| | secretory sperm binding protein; | | |
| | lymphocyte antigen T1/Leu-1; T1; | | |
| | LEU1; CD5 molecule | | |
| CD7 | T-cell antigen CD7; CD7 antigen | P09564 | NM_006137 |
| | (p41); T-cell leukemia antigen; T-cell | | |
| | surface antigen Leu-9; p41 protein; | | |
| | GP40; TP41; Tp40; LEU-9; CD7 | | |
| | molecule | | |
| CD38 | ADP-ribosyl cyclase/cyclic ADP- | P28907 | NM_001775 |
| | ribose hydrolase 1; 2′-phospho-ADP- | | |
| | ribosyl cyclase; 2′-phospho-cyclic- | | |
| | ADP-ribose transferase; ADP-ribosyl | | |
| | cyclase 1; CD38 antigen (p45); | | |
| | NAD(+) nucleosidase; cluster of | | |
| | differentiation 38; cyclic ADP-ribose | | |
| | hydrolase 1; ecto-nicotinamide | | |
| | adenine dinucleotide glycohydrolase; | | |
| | ADPRC1; ADPRC 1; CD38 molecule | | |
| CD30 | tumor necrosis factor receptor | P28908 | NM_001243 |
| | superfamily member 8; CD30L | | NM_001281430 |
| | receptor; Ki-1 antigen; cytokine | | |
| | receptor CD30; lymphocyte | | |
| | activation antigen CD30; CD30; Ki- | | |
| | 1; D1S166E; TNFRSF8 | | |

Solid Tumors

| | | | |
|---|---|---|---|
| B7-H3 | CD276 antigen; B7 homolog 3; | Q5ZPR3 | NM_001024736 |
| | costimulatory molecule; B7H3; B7- | | NM_001329628 |
| | H3; B7RP-2; 4Ig-B7-H3; CD276 | | NM_001329629 |
| | molecule | | NM_025240 |
| HER2 | receptor tyrosine-protein kinase | P04626 | NR_110535.2 |
| | erbB-2; c-erb B2/neu protein; | | NM_001382782.1 |
| | herstatin; human epidermal growth | | NM_001289936.2 |
| | factor receptor 2; metastatic lymph | | NM_001005862.3 |
| | node gene 19 protein; | | NM_001289938.2 |
| | neuro/glioblastoma derived oncogene | | XM_024450643.1 |
| | homolog; | | XM_024450642.1 |
| | neuroblastoma/glioblastoma derived | | XM_024450641.1 |
| | oncogene homolog; proto-oncogene | | NM_001382783.1 |
| | Neu; proto-oncogene c-ErbB-2; | | NM_001382787.1 |
| | tyrosine kinase-type cell surface | | NM_001382784.1 |
| | receptor HER2; v-erb-b2 avian | | NM_001382786.1 |
| | erythroblastic leukemia viral | | NM_001382789.1 |
| | oncogene homolog 2; v-erb-b2 avian | | NM_001382788.1 |
| | erythroblastic leukemia viral | | NM_001382785.1 |
| | oncoprotein 2; v-erb-b2 | | NM_004448.4 |
| | erythroblastic leukemia viral | | NM_001289937.2 |
| | oncogene homolog 2; | | NM_001382796.1 |
| | neuro/glioblastoma derived oncogene | | NM_001382798.1 |
| | homolog; NEU; NGL; HER2; TKR1; | | NM_001382800.1 |
| | CD340; HER-2; VSCN2; MLN 19; | | NM_001382797.1 |
| | HER-2/neu; erb-b2 receptor tyrosine | | NM_001382805.1 |
| | kinase 2; ERBB2 | | NM_001382792.1 |
| | | | NM_001382793.1 |
| | | | NM_001382803.1 |
| | | | NM_001382794.1 |
| | | | NM_001382795.1 |
| | | | NM_001382801.1 |
| | | | NM_001382790.1 |
| | | | NM_001382806.1 |
| | | | NM_001382802.1 |
| | | | NM_001382799.1 |
| | | | NM_001382791.1 |
| | | | NM_001382804.1 |
| CD44v6 | CD44 molecule variant 6; CD44 | P16070-6 | NM_001202555 |
| | antigen variant 6; CD44 molecule | | |
| | isoform 6; | | |
| CEA | carcinoembryonic antigen-related cell | P06731 | NM_004363.6 |
| | adhesion molecule 5; | | NM_001291484.3 |
| | carcinoembryonic antigen related cell | | XM_017026145.2 |
| | adhesion molecule 5; meconium | | XM_017026146.2 |
| | antigen 100; CEA; CD66e; CEA cell | | XM_011526322.2 |
| | adhesion molecule 5; CEACAM5 | | NM_001308398.2 |
| CD133 | prominin-1; antigen AC133; | O43490 | NM_001145847 |
| | hProminin; hematopoietic stem cell | | NM_001145848 |
| | antigen; prominin-like protein 1; | | NM_001145849 |
| | RP41; AC133; CD133; MCDR2; | | NM_001145850 |
| | STGD4; CORD12; PROML1; | | NM_001145851 |
| | MSTP061 | | NM_001145852 |
| | | | NM_001371406 |
| | | | NM_001371407 |
| | | | NM NM_006017 |
| | | | _001371408 |
| c-Met | hepatocyte growth factor receptor; | P08581 | XR_001744772.1 |
| | HGF receptor; HGF/SF receptor; SF | | NM_001127500.3 |
| | receptor; proto-oncogene c-Met; | | NM_000245.4 |
| | scatter factor receptor; tyrosine- | | NM_001324402.2 |
| | protein kinase Met; HGFR; AUTS9; | | NM_001324401.3 |
| | RCCP2; c-Met; DFNB97; MET | | XM_011516223.1 |
| | proto-oncogene, receptor tyrosine | | XM_006715990.2 |
| | kinase; MET | | |
| EGFRvIII | epidermal growth factor receptor; | P00533 | NM_005228.5 |
| | avian erythroblastic leukemia viral | | NM_001346899.2 |
| | (v-erb-b) oncogene homolog; cell | | NM_001346941.2 |
| | growth inhibiting protein 40; cell | | NM_001346898.2 |
| | proliferation-inducing protein 61; | | NM_001346897.2 |
| | epidermal growth factor receptor | | NM_201284.2 |
| | tyrosine kinase domain; erb-b2 | | NM_201282.2 |
| | receptor tyrosine kinase 1; proto- | | NM_201283.2 |
| | oncogene c-ErbB-1; receptor | | NM_001346900.2 |
| | tyrosine-protein kinase erbB-1; | | |
| | ERBB; ERRP; HER1; mENA; | | |
| | ERBB1; PIG61; NISBD2; epidermal | | |
| | growth factor receptor; EGFR; | | |
| | EGFRvIII | | |
| Epcam | epithelial cell adhesion molecule; | P16422 | NM_002354 |
| | adenocarcinoma-associated antigen; | | |
| | cell surface glycoprotein Trop-1; | | |
| | epithelial glycoprotein 314; human | | |
| | epithelial glycoprotein-2; major | | |
| | gastrointestinal tumor-associated | | |
| | protein GA733-2; membrane | | |
| | component, chromosome 4, surface | | |
| | marker (35kD glycoprotein); | | |
| | trophoblast cell surface antigen 1; | | |
| | tumor-associated calcium signal | | |
| | transducer 1; ESA; KSA; M4S1; | | |
| | MK-1; DIAR5; EGP-2; EGP40, | | |
| | KS1/4; MIC18; TROP1; EGP314; | | |
| | HNPCC8; TACSTD1; EPCAM | | |
| EphA2 | ephrin type-A receptor 2; epithelial | P29317 | NM_001329090 |
| | cell receptor protein tyrosine kinase; | | NM_004431 |
| | soluble EPHA2 variant 1; tyrosine- | | |
| | protein kinase receptor ECK; ECK; | | |
| | CTPA; ARCC2; CTPP1; CTRCT6; | | |
| | EPH receptor A2; EPHA2 | | |
| FR-alpha | folate receptor alpha; FR-alpha; KB | P15328 | NM_016724.3 |
| | cells FBP; adult folate-binding | | NM_016725.3 |
| | protein; folate binding protein; folate | | NM_000802.3 |
| | receptor 1 (adult); folate receptor, | | NM_016729.3 |
| | adult; ovarian tumor-associated | | |
| | antigen MOv18; FBP; FOLR; | | |
| | NCFTD; FRalpha; FOLR1 | | |
| GD2 | | | |
| GPC3 | glypican-3; glypican proteoglycan 3; | P51654 | NM_001164619.2 |
| | heparan sulphate proteoglycan; | | NM_001164618.2 |
| | intestinal protein OCI-5; secreted | | NM_004484.4 |
| | glypican-3; SGB; DGSX; MXR7; | | NM_001164617.2 |
| | SDYS; SGBS; OCI-5; SGBS1; | | XM_017029413.2 |
| | GTR2-2; GPC3; glypican 3 | | |
| IL-13R-alpha2 | interleukin-13 receptor subunit alpha- | Q14627 | NM_000640 |
| | 2; IL-13 receptor subunit alpha-2; IL- | | |
| | 13R subunit alpha-2; IL-13R-alpha- | | |
| | 2; IL-13RA2; cancer/testis antigen | | |
| | 19; interleukin 13 binding protein; | | |
| | interleukin 13 receptor alpha 2 chain; | | |
| | interleukin 13 receptor, alpha 2; | | |
| | CT19; IL-13R; IL13BP; CD213A2; | | |
| | IL13RA2 | | |
| IL-11R-alpha | interleukin-11 receptor subunit alpha; | Q14626 | NM_001142784 |
| | IL-11 receptor subunit alpha; IL-11R | | |
| | subunit alpha; interleukin 11 | | |
| | receptor, alpha; interleukin-11 | | |
| | receptor alpha chain; CRSDA; | | |
| | IL11RA | | |

In some embodiments, the antigen recognition domain of the CAR described herein binds (e.g. specifically binds) to at least one of L1-CAM, Mesothelin, MUC1, MUC16, NKGD2, PSCA, PSMA, ROR1 and ALK. The antigen specific CAR, when expressed on the cell surface, redirects the specificity of immune cells (e.g. T cells) to the respective antigen.

In some embodiments, the antigen recognition domain of a CAR described herein binds (e.g., specifically binds) to CD22. The CD22-specific CAR, when expressed on the cell surface, redirects the specificity of T cells to human CD22 (see, e.g., Accession Nos. NM_001185099; NM_001185100; NM_001185101; NM_001278417 and NP_001172028; NP_001172029; NP_001172030; NP_001265346; NP_001762).

In some embodiments, the antigen recognition domain of a CAR described herein binds (e.g., specifically binds) to CD19. The CD19-specific CAR, when expressed on the cell surface, redirects the specificity of T cells to human CD19 (see, e.g., Accession Nos. NM_001178098; NM_001770; NM_001385732 and NP_001171569; NP_001761).

### i) Antigen Recognition Domains Comprising an Anti-CD22 Antibody or Fragment Thereof

In some embodiments, the antigen recognition domain of a CAR provided herein comprises an antibody or an antigen-binding fragment thereof. In some embodiments, the antigen recognition domain of a CAR provided herein comprises a single chain antibody fragment (scFv) comprising a light chain variable domain (VL) and heavy chain variable domain (VH) of a monoclonal anti-CD22 antibody. Optionally, the VH and VL may be joined by a flexible linker, such as a glycine-serine linker or a Whitlow linker. In some embodiments, the antigen binding moiety may comprise VH and VL that are directionally linked, for example, from N to C terminus, VH-linker-VL or VL-linker-VH.

In some embodiments, the antigen recognition domain of a CAR provided herein comprises an scFv whose affinity for CD22 has been optimized to induce cytotoxicity of tumor cells that produce high levels or normal levels of CD22. In some embodiments, the antigen recognition domain of a CAR provided herein comprises an scFv whose affinity for CD22 has been optimized to induce cytotoxicity of tumor cells that produce low levels of CD22.

Exemplary anti-CD22 scFvs from which antigen recognition domains for use in a CAR described herein may be derived include, but are not limited to, m971 and immunologically active and/or antigen-binding fragments thereof. Thus, in some embodiments, the antigen recognition domain of a CAR provided herein comprises a VH and VL derived from any one of the anti-CD22 antibodies m971.

In some embodiments, the antigen recognition domain of a CAR described herein comprises complementarity determining regions (CDRs) and/or a heavy chain variable domain (VH) and a light chain variable domain (VL) derived from the anti-CD22 antibody m971. The m971 antibody comprises a VH comprising the amino acid sequence of SEQ ID NO 70 and a VL comprising the amino acid sequence of SEQ ID NO: 71. The amino acid sequences of the VH (and corresponding CDRH1, CDRH2, and CDRH3) and VL (and corresponding CDRL1, CDRL2, and CDRL3) of m971 are provided below:

M971-VH:
(SEQ ID NO: 70)
QVQLQQSGPGLVKPSQTLSLTCAISGDSVSSNSAAWNWIRQSPSRGLEW
LGRTYYRSKWYNDYAVSVKSRITINPDTSKNQFSLQLNSVTPEDTAVYY
CAREVTGDLEDAFDIWGQGTMVTVSS
M971-VL:
(SEQ ID NO: 71)
DIQMTQSPSSLSASVGDRVTITCRASQTIWSYLNWYQQRPGKAPNLLIY
AASSLQSGVPSRFSGRGSGTDFTLTISSLQAEDFATYYCQQSYSIPQTF
GQGTKLEIK
M971-CDRH1:
(SEQ ID NO: 72)
GDSVSSNSAA
M971-CDRH2:
(SEQ ID NO: 73)
TYYRSKWYN
M971-CDRH3:
(SEQ ID NO: 74)
AREVTGDLEDAFDI
M971-CDRL1:
(SEQ ID NO: 75)
QTIWSY
M971-CDRL2:
AAS
M971-CDRL3:
(SEQ ID NO: 77)
QQSYSIPQT

In some embodiments, the antigen recognition domain of a CAR described herein comprises an scFv comprising a VH and a VL, wherein the VH comprises a CDRH1 of SEQ ID NO: 72, a CDRH2 of SEQ ID NO: 73, and a CDRH3 of SEQ ID NO: 74, and the VL comprises a CDRL1 of SEQ ID NO: 75, a CDRL2 of amino acid sequence AAS, and a CDRL3 of SEQ ID NO: 77. In some embodiments, the antigen recognition domain of a CAR described herein comprising a VH and a VL, wherein the VH comprises the amino acid sequence of SEQ ID NO: 70, and the VL comprises the amino acid sequence of SEQ ID NO: 71.

The antigen recognition domain of the CARs provided herein may include CDRs and/or VH and VL derived from an anti-CD22 antibody (or antigen binding fragment thereof) Anti-CD22 antibodies of the disclosure can comprise any one of the partial light chain sequences known in the art and/or any one of partial heavy chain sequences known in the art. In some embodiments, the antigen recognition domain of a CAR described herein comprises an scFv comprising a VH and a VL, wherein the VH comprises the amino acid sequence of a VH from an anti-CD22 antibody known in the art, and the VL comprises the amino acid sequence of the corresponding VL known in the art.

In some embodiments, the antigen recognition domain of a CAR described herein comprises an scFv comprising a VH and a VL, wherein the VH comprises a CDRH1, a CDRH2, and a CDRH3 each comprising the amino acid sequence of a CDRH1, a CDRH2, and a CDRH3 of an anti-CD22 antibody known in the art, and wherein and the VL comprises a CDRL1, a CDRL2, and a CDRL3 each comprising the amino acid sequence of a CDRL1, a CDRL2, and a CDRL3 of the same anti-CD22 antibody known in the art. Determination of CDR regions is well within the skill of the art. It is understood that in some embodiments, CDRs can be a combination of the Kabat and Chothia CDR (also termed “combined CRs” or “extended CDRs”).

In some embodiments, the CDRs are the Kabat CDRs. In other embodiments, the CDRs are the Chothia CDRs. In other embodiments, the CDRs are IMGT CDRs. In other words, in embodiments with more than one CDR, the CDRs may be any of Kabat, Chothia, IMGT combination CDRs, or combinations thereof.

### ii) Antigen Recognition Domains Comprising an Anti-CD19 Antibody or Fragment Thereof

In some embodiments, the antigen recognition domain of a CAR provided herein comprises an scFv whose affinity for CD19 has been optimized to induce cytotoxicity of tumor cells that produce high levels or normal levels of CD19. In some embodiments, the antigen recognition domain of a CAR provided herein comprises an scFv whose affinity for CD19 has been optimized to induce cytotoxicity of tumor cells that produce low levels of CD19. Illustrative examples of such affinity tuning are provided in Caruso et al. (2015) Cancer Res. 75: 3505-18 and Liu et al. (2015) Cancer Res. 75: 3596-607.

In some embodiments, the antigen recognition domain of a CAR provided herein comprises an antibody or an antigen-binding fragment thereof. In some embodiments, the antigen recognition domain of a CAR provided herein comprises a single chain antibody fragment (scFv) comprising a light chain variable domain (VL) and heavy chain variable domain (VH) of a monoclonal anti-CD19 antibody. Optionally, the VH and VL may be joined by a flexible linker, such as a glycine-serine linker or a Whitlow linker. In some embodiments, the scFv is humanized. In some embodiments, the antigen binding moiety may comprise VH and VL that are directionally linked, for example, from N to C terminus, VH-linker-VL or VL-linker-VH.

In some embodiments, the antigen recognition domain of a CAR provided herein comprises an scFv whose affinity for CD19 has been optimized to induce cytotoxicity of tumor cells that produce high levels or normal levels of CD19. In some embodiments, the antigen recognition domain of a CAR provided herein comprises an scFv whose affinity for CD19 has been optimized to induce cytotoxicity of tumor cells that produce low levels of CD19.

Exemplary anti-CD19 scFvs from which antigen recognition domains for use in a CAR described herein may be derived include, but are not limited to, FMC63 and immunologically active and/or antigen-binding fragments thereof. Thus, in some embodiments, the antigen recognition domain of a CAR provided herein comprises a VH and VL derived from any one of the anti-CD19 antibodies FMC63.

Exemplary anti-CD19 scFvs from which antigen recognition domains for use in a CAR described herein may be derived include, but are not limited to, inebilizumab (MEDI-551), MDX-1342, tafasitamab, obexelimab, B4 (Merck), hA19 (immunomedics), and immunologically active and/or antigen-binding fragments thereof. Thus, in some embodiments, the antigen recognition domain of a CAR provided herein comprises a VH and VL derived from any one of these anti-CD19 antibodies.

In some embodiments, the antigen recognition domain of a CAR described herein comprises complementarity determining regions (CDRs) and/or a heavy chain variable domain (VH) and a light chain variable domain (VL) derived from the anti-CD19 antibody FMC63. The FMC63 antibody comprises a VH comprising the amino acid sequence of SEQ ID NO: 78 and a VL comprising the amino acid sequence of SEQ ID NO: 79 The amino acid sequences of the VH (and corresponding CDRH1, CDRH2, and CDRH3) and VL, (and corresponding CDRL1, CDRL2, and CDRL3) of FMC63 are provided below:

FMC63-VH:
(SEQ ID NO: 78)
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTV
FMC63-VL:
(SEQ ID NO: 79)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEIT
FMC63-CDRH1:
(SEQ ID NO: 80)
GVSLPDYG
FMC63-CDRH2:
(SEQ ID NO: 81)
IWGSETT
FMC63-CDRH3:
(SEQ ID NO: 82)
AKHYYYGGSYAMDY
FMC63-CDRL1:
(SEQ ID NO: 83)
QDISKY
FMC63-CDRL2:
HTS
FMC63-CDRL3:
(SEQ ID NO: 85)
QQGNTLPYT

In some embodiments, the antigen recognition domain of a CAR described herein comprises an scFv comprising a VH and a VL, wherein the VH comprises a CDRH1 of SEQ ID NO: 80, a CDRH2 of SEQ ID NO: 81, and a CDRH3 of SEQ ID NO; 82, and the VL comprises a CDRL1 of SEQ ID NO: 83, a CDRL2 of amino acid sequence HTS, and a CDRL3 of SEQ ID NO: 85. In some embodiments, the antigen recognition domain of a CAR described herein comprising a VH and a VL, wherein the VH comprises the amino acid sequence of SEQ ID NO: 78, and the VL comprises the amino acid sequence of SEQ ID NO: 79.

The antigen recognition domain of the CARs provided herein may include CDRs and/or VH and VL derived from an anti-CD19 antibody (or antigen binding fragment thereof). Anti-CD19 antibodies of the disclosure can comprise any one of the partial light chain sequences known in the art and/or any one of partial heavy chain sequences known in the art. In some embodiments, the antigen recognition domain of a CAR described herein comprises an scFv comprising a VH and a VL, wherein the VH comprises the amino acid sequence of a VH from an anti-CD19 antibody known in the art, and the VL comprises the amino acid sequence of the corresponding VL from an anti-CD19 antibody known in the art.

In some embodiments, the antigen recognition domain of a CAR described herein comprises an scFv comprising a VH and a VL, wherein the VH comprises a CDRH1, a CDRH2, and a CDRH3 each comprising the amino acid sequence of a CDRH1, a CDRH2, and a CDRH3 of an anti-CD19 antibody known in the art, and wherein and the VL comprises a CDRL1, a CDRL2, and a CDRL3 each comprising the amino acid sequence of a CDRL1, a CDRL2, and a CDRL3 of the same anti-CD19 antibody known in the art. Determination of CDR regions is well within the skill of the art. It is understood that in some embodiments, CDRs can be a combination of the Kabat and Chothia CDR (also termed “combined CRs” or “extended CDRs”).

In some embodiments, the CDRs are the Kabat CDRs. In other embodiments, the CDRs are the Chothia CDRs. In other embodiments, the CDRs are IMGT CDRs. In other words, in embodiments with more than one CDR, the CDRs may be any of Kabat, Chothia, IMGT combination CDRs, or combinations thereof.

### B. Signal Peptides

In some embodiments, any of the CARS provided herein comprises a signal peptide (also known as a signal peptide, signal sequence, signal peptide sequence, leader peptide, and leader peptide sequence). In some embodiments, the antigen recognition domain of the CAR described herein comprises a signal peptide or a leader peptide sequence. Exemplary signal sequences include but are not limited to a CD8a signal sequence or an IgG signal sequence. In some embodiments, the CAR described herein does not comprise a signal peptide. In some embodiments, the T cell or populations of T cells provided herein comprise a CAR comprising a signal peptide. In some embodiments, the T cell or populations of T cell provided herein comprise a CAR that does not comprise a signal peptide.

In some embodiments, the CAR (e.g., the antigen recognition domain of the CAR) may comprise a human CD8a signal sequence comprising an amino acid sequence having at least 90%, 91%, 92%, 93%, 94%, 95%, 96%, 97%, 98%, 99% or 100% identity with the amino acid sequence of SEQ ID NO: 1.

In some embodiments, the CAR (e.g., the antigen recognition domain of the CAR) may comprise a human CD8a signal sequence comprising an amino acid sequence having at least 90%, 91%, 92%, 93%, 94%, 95%, 96%, 97%, 98%, 99% or 100% identity with the amino acid sequence of SEQ ID NO 2.

In some embodiments, the CAR (e.g., the antigen recognition domain of the CAR) may comprise a human IgG signal sequence comprising an amino acid sequence having at least 90%, 91%, 92%, 93%, 94%, 95%, 96%, 97%, 98%, 99% or 100%1 identity with the amino acid sequence of SEQ ID NO. 3.

In some embodiments, the CAR (e.g., the antigen recognition domain of the CAR) may comprise a human IgG signal sequence comprising an amino acid sequence having at least 90%, 91%, 92%, 93%, 94%, 95%, 96%, 97%, 98%, 99% or 100%1 identity with the amino acid sequence of SEQ ID NO. 4.

### C. Hinge Domains

In some embodiments, a hinge domain (also known as a spacer region or a stalk region) is located between the antigen recognition domain and the transmembrane domain of the CAR In particular, stalk regions are used to provide more flexibility and accessibility for the extracellular antigen recognition domain. In some embodiments, a hinge domain may comprise up to about 300 amino acids. In some embodiments, the hinge comprises about 10 to about 100 amino acids in length. In some embodiments, the hinge comprises about 25 to about 50 amino acids in length. In some embodiments, the hinge domain establishes an optimal effector-target inter membrane distance. In some embodiments, the hinge domain provides flexibility for antigen recognition domain to bind the target antigen. Any protein that is stable and/or dimerizes can serve this purpose.

A hinge domain may be derived from all or part of naturally occurring molecules, such as from all or part of the extracellular region of CD8, CD8α, CD4, CD28, 4-1BB, or IgG (in particular, the hinge domain of an IgG, for example from IgG1, IgG2 or IgG4), or from all or part of an antibody heavy-chain constant region. Alternatively, the hinge domain may be a synthetic sequence that corresponds to a naturally occurring hinge sequence, or may be an entirely synthetic hinge sequence. In some embodiments, it corresponds to Fc domains of a human immunoglobulin, e.g., either the CH2 or CH3 domain. In some embodiments, the CH2 and CH3 hinge domain of a human immunoglobulin that has been modified to improve dimerization. In some embodiments, the hinge is a hinge portion of an immunoglobulin. In some embodiments, the hinge domain comprises a CH3 region of a human immunoglobulin. In some embodiments, the hinge domain comprises a CH2 and CH3 region of a human immunoglobulin. In some embodiments, the CH2 region comprises a human IgG1, IgG2 or IgG4 immunoglobulin CH2 region.

In some embodiments, the hinge domain is a part of human CD8α chain (e.g., NP_001139345.1) In some embodiments, the hinge domain of CARs described herein comprises a subsequence of CD8α, CD28, or the constant region of an immunoglobulin (e.g. IgG1, IgG2, IgG3, IgG4) either in wild-type form or mutated to avoid Fc-receptor binding in particular the hinge domain of any of an CD8α, or a CD28 In some embodiments, the stalk region comprises a human CD8α hinge, or a human CD28 hinge.

In some embodiments, the hinge may comprise or consist of a human CD8α hinge domain comprising an amino acid sequence having at least 90%, 91%, 92%, 93%, 94%, 95%, 96%, 97%, 98%, 99% or 100% identity with the amino acid sequence of SEQ ID NO: 5.

In some embodiments, the hinge may comprise or consist of a human CD8α hinge domain comprising an amino acid sequence having at least 90%, 91%, 92%, 93%, 94%, 95%, 96%, 97%, 98%, 99% or 100% identity with the amino acid sequence of SEQ ID NO: 6.

In some embodiments, the hinge may comprise or consist of a human CD28 hinge domain comprising an amino acid sequence having at least 90%, 91%, 92%, 93%, 94%, 95%, 96%, 97%, 98%, 99% or 100% identity with the amino acid sequence of SEQ ID NO: 7.

In some embodiments, the hinge may comprise or consist of a human CD28 hinge domain comprising an amino acid sequence having at least 90%, 91%, 92%, 93%, 94%, 95%, 96%, 97%, 98%, 99% or 100% identity with the amino acid sequence of SEQ ID NO: 8.

### D. Transmembrane Domains

Suitable transmembrane domains for a CAR disclosed herein have the ability to (a) be expressed at the surface of a cell, which is in some embodiments an immune cell such as, for example a T cell, and/or (b) interact with the ligand-binding domain and intracellular signaling domain for directing cellular response of an immune cell against a predefined target cell. The transmembrane domain can be derived either from a natural or from a synthetic source. The transmembrane domain can be derived from any membrane-bound or transmembrane protein. As non-limiting examples, the transmembrane domains can include the transmembrane region(s) of alpha, beta, delta, or gamma of the T-cell receptor; or a transmembrane region from CD8, CD8α, CD8 beta, CD28, CD3-epsilon, CD3-delta, CD3-gamma, CD3z, CD4, 4-1BB, OX40, ICOS, PD-1, LAG-3, 2B4 or BTLA transmembrane domain or a portion of any of the foregoing or a combination of any of the foregoing. In some embodiments, the transmembrane domain comprises a CD8α transmembrane domain. In some embodiments, the transmembrane domain comprises a CD28 transmembrane domain.

Alternatively, the transmembrane domain can be synthetic, and can comprise hydrophobic residues such as leucine and valine. In some embodiments, a triplet of phenylalanine, tryptophan and valine is found at one or both termini of a synthetic transmembrane domain Optionally, a short oligonucleotide or polypeptide linker, in some embodiments, between 2 and 10 amino acids in length may form the linkage between the transmembrane domain and the intracellular domain of a CAR. In some embodiments, the linker is a glycine-serine linker.

In some embodiments, the transmembrane domain of a CAR provided herein may comprise or consist of a human CD8α transmembrane domain comprising an amino acid sequence having at least 90%, 91%, 92%, 93%, 94%, 95%, 96%, 97%, 98%, 99% or 100% identity with the amino acid sequence of SEQ ID NO: 13.

In some embodiments, the transmembrane domain of a CAR provided herein may comprise or consist of a human CD28 transmembrane domain comprising an amino acid sequence having at least 90%, 91%, 92%, 93%, 94%, 95%, 96%, 97%, 98%, 99% or 100% identity with the amino acid sequence of SEQ ID NO: 14.

In some embodiments, the transmembrane domain of a CAR provided herein may comprise or consist of a human CD3ε transmembrane domain comprising an amino acid sequence having at least 90, 91%, 92%, 93%, 94%, 95%, 96%, 97%, 98%, 99% or 10% identity with the amino acid sequence of SEQ 1D NO: 15.

### E. Costimulatory Domains

The intracellular domain of a CAR provided herein may comprise one or more costimulatory domains. Exemplary costimulatory domains include, but are not limited to a 4-1 BB (CD137), CD28, CD97, CD11a-CD18, CD2, ICOS, CD27, CD154, CD8α, OX40 (CD134), ZAP40, CD30, GITR, HVEM, DAP10, DAP12, MyD88, 2B4 costimulatory domain, or a fragment thereof, or a combination thereof. In some instances, a first CAR described herein comprises one or more, or two or more of costimulatory domains selected from a 4-1BB (CD137), CD28, CD97, CD11a-CD18, CD2, ICOS, CD27, CD154, CD8α, OX40 (CD134), ZAP40, CD30, GITR, HVEM, DAP10, DAP12, MyD88, 2B4 costimulatory domain, or a fragment thereof, or a combination thereof. In some embodiments, a CAR described herein comprises a CD28 costimulatory domain or a fragment thereof. In some embodiments, a CAR described herein comprises a 4-1BB (CD137) costimulatory domain or a fragment thereof.

In some embodiments, the costimulatory domain of a CAR provided herein may comprise or consist of a human CD28 costimulatory domain comprising an amino acid sequence having at least 90%, 91%, 92%, 93%, 94%, 95%, 96%, 97%, 98%, 99% or 100% identity with the amino acid sequence of SEQ ID NO: 16.

In some embodiments, the costimulatory domain of a CAR provided herein may comprise or consist of a human CD28 costimulatory domain comprising an amino acid sequence having at least 90%, 91%, 92%, 93%, 94%, 95%, 96%, 97%, 98%, 99% or 100% identity with the amino acid sequence of SEQ ID NO: 17.

In some embodiments, the costimulatory domain of a CAR provided herein may comprise or consist of a human 4-1BB costimulatory domain comprising an amino acid sequence having at least 90%, 91%, 92%, 93%, 94%, 95%, 96%, 97%, 98%, 99% or 100% identity with the amino acid sequence of SEQ ID NO: 18.

### F. Activation Domain

In some embodiments, the activation domain of a CAR disclosed herein is responsible for activation of at least one of the normal effector functions of the immune cell (e.g. T cell) in which the CAR is expressed. The terms “intracellular signaling domain” or “intracellular domain” are used interchangeably and refer to a domain that comprises a co-stimulatory domain and/or an activation domain. The term “effector function” refers to a specialized function of a cell. Effector function of a T-cell, for example, may be cytolytic activity or helper activity including the secretion of cytokines. The term “activation domain” refers to the portion of a protein which transduces the effector function signal and directs the cell to perform a specialized function. While usually an entire activation domain can be employed, in many cases it is not necessary to use the entire chain. To the extent that a truncated portion of the activation domain is used, such truncated portion may be used in place of the intact chain as long as it transduces the effector function signal. The term activation domain is thus meant to include any truncated portion of the activation domain sufficient to transduce the effector function signal. In some embodiments, the activation domain further comprises a signaling domain for T-cell activation. In some instances, the signaling domain for T-cell activation comprises an intracellular domain derived from CD3ζ (CD3zeta; CD3z) or an intracellular domain derived from CD3ε. In some embodiments, the CAR described herein comprises at least one (e.g., one, two, three, or more) activation domains selected from a CD3ζ or CD3ε, or a portion of any of the foregoing. In some embodiments, the CAR described herein has an activation domain comprising a domain derived from CD3ζ (CD3zeta; CD3z). In some embodiments, the CAR described herein has an activation domain comprising a domain derived from CD3ε.

In some embodiments, the activation domain of a CAR described herein may comprise or consist of a CD3zeta activation (intracellular signaling) domain (e.g., a human CD3zeta activation domain) comprising an amino acid sequence having at least 90%, 91%, 92%, 93%, 94%, 95%, 96%, 97%, 98%, 99% or 100% identity with the amino acid sequence of SEQ D NO. 25.

In some embodiments, the activation domain of a CAR described herein may comprise or consist of a CD3zeta activation (intracellular signaling) domain (e.g., a human CD3zeta activation domain) comprising an amino acid sequence having at least 90%, 91%, 92%, 93%, 94%, 95%, 96%, 97%, 98%, 99% or 100% identity with the amino acid sequence of SEQ ID NO: 26.

In some embodiments, the CD3zeta activation domain comprises a mutation in an ITAM domain. Examples of mutations in ITAM domains of CD3zeta are provided in Feucht et al., Nat Med. 2019; 25(1): 82-88. In some embodiments, each of the two tyrosine residues in one or more of ITAM1, ITAM2, or ITAM3 domains of the CD3zeta activation domain are point-mutated to a phenylalanine residue. In some embodiments, the CD3zeta activation domain comprises a deletion of one or more of the ITAM1, ITAM2, or ITAM3 domains.

In some embodiments, the activation domain of a CAR described herein may comprise or consist of a CD3ε intracellular signaling domain comprising an amino acid sequence having at least 90%, 91%, 921%, 93%, 94%, 95%, 96%, 97%, 98%, 99% or 100% identity with the amino acid sequence of any one of SEQ ID NOs: 27.

In some embodiments, the activation domain of a CAR described herein may comprise or consist of a CD3ε intracellular signaling domain comprising an amino acid sequence having at least 90%, 91%, 92%, 93%, 94%, 95%, 96%, 97%, 98%, 99% or 100% identity with the amino acid sequence of any one of SEQ ID NOs: 28.

Included in the scope of the invention are nucleic acid sequences that encode functional portions of the CAR described herein. Functional portions encompass, for example, those parts of a CAR, that retain the ability to recognize target cells, or detect, treat, or prevent a disease, to a similar extent, the same extent, or to a higher extent, as the parent CAR

In embodiments, the CARs described herein contain additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity of the CAR, as compared to the biological activity of the parent CAR.

The term “functional variant,” as used herein in reference to a CAR, refers to a CAR, a polypeptide, or a protein having substantial or significant sequence identity or similarity to the CAR encoded by a nucleic acid sequence, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to a nucleic acid sequence encoding the parent CAR, a nucleic acid sequence encoding a functional variant of the CAR can be for example, about 10% identical, about 25% identical, about 30% identical, about 50% identical, about 65% identical, about 80% identical, about 90% identical, about 95% identical, or about 99%, identical to the nucleic acid sequence encoding the parent CAR.

A CAR described herein include (including functional portions and functional variants thereof) glycosylated, amidated, carboxylated, phosphorylated, esterified, N-acylated, cyclized via, e.g., a disulfide bridge, or converted into an acid addition salt and/or optionally dimerized or polymerized.

Table 2 provides exemplary amino acid sequences of the domains which can be used in the CARs described herein. In some embodiments, a CAR provided herein comprises one or more domains described in Table 8, or a fragment or portion thereof.

TABLE 2
Exemplary Amino Acid Sequences of CAR Domains

| | | |
|---|---|---|
| | | SEQ ID |
| Exemplary CAR domains | Amino Acid Sequence | NO: |

SIGNAL PEPTIDE

| | | |
|---|---|---|
| human CD8alpha signal | ASATMALPVTALLLPLALLLHAARP | 1 |
| sequence | | |
| human CD8alpha signal | MALPVTALLLPLALLLHAARP | 2 |
| sequence | | |
| human IgG heavy chain signal | GSMEFGLSWLFLVAILKGVQCSR | 3 |
| sequence | | |
| human IgG heavy chain signal | MEFGLSWLFLVAILKGVQCSR | 4 |
| sequence | | |

HINGES

| | | |
|---|---|---|
| human CD8alpha hinge | LETTTPAPRPPTPAPTIASQPLSLRPEACRPAAGGAV | 5 |
| domain | HTRGLDFACD | |
| human CD8alpha hinge | TTTPAPRPPTPAPTIASQPLSIRPEACRPAAGGAVHT | 6 |
| domain | RGLDFACD | |
| human CD28 hinge domain | SRIEVMYPPPYLDNEKSNGTIIHVKGKHLCPSPLFPG | 7 |
| | PSKP | |
| human CD28 hinge domain | IEVMYPPPYLDNEKSNGTIIHVKGKHLCPSPLFPGPS | 8 |
| | KP | |
| human IgG1 hinge domain | EPKSCDKTHTCPPCPAPELLGGPSVFLFPPKPKDTLM | 9 |
| | ISRTPEVTCVVVDVSHEDPEVKFNWYVDGVEVHNAKT | |
| | KPREEQYNSTYRVVSVLTVLHQDWLNGKEYKCKVSNK | |
| | ALPAPIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNHYT | |
| | QKSLSLSPGK | |
| human IgG1 hinge domain | EPKSCDKTHTCP | 10 |
| human IgG4 hinge domain | ESKYGPPCPSCPAPEFLGGPSVFLFPPKPKDTLMISR | 11 |
| | TPEVTCVVVDVSQEDPEVQFNWYVDGVEVHNAKTKPR | |
| | EEQFNSTYRVVSVLTVLHQDWLNGKEYKCKVSNKGLP | |
| | SSIEKTISKAKGQPREPQVYTLPPSQEEMTKNQVSLT | |
| | CLVKGFYPSDIAVEWESNGQPENNYKTTPPVLDSDGS | |
| | FFLYSRLTVDKSRWQEGNVFSCSVMHEALHNHYTQKS | |
| | LSLSLGK | |
| human IgG4 hinge domain | ESKYGPPCPSCP | 12 |

TRANSMEMBRANE

| | | |
|---|---|---|
| human CD8alpha | IYIWAPLAGTCGVLLLSLVITLYC | 13 |
| transmembrane domain | | |
| human CD28 transmembrane | FWVLVVVGGVLACYSLLVTVAFIIFWV | 14 |
| domain | | |
| human CD3ε transmembrane | VMSVATIVIVDICITGGLLLLVYYWS | 15 |
| domain | | |
| human 2B4 transmembrane | FLVIIVILSALFLGTLACFCV | 91 |
| domain | | |

COSTIMULATORY DOMAINS

| | | |
|---|---|---|
| human CD28 costimulatory | RSKRSRGGHSDYMNMTPRRPGPTRKHYQPYAPPRDFA | 16 |
| domain | AYRS | |
| human CD28 costimulatory | RSKRSRLLHSDYMNMTPRRPGPTRKHYQPYAPPRDFA | 17 |
| domain | AYRS | |
| human 4-1BB costimulatory | KRGRKKLLYIFKQPFMRPVQTTQEEDGCSCRFPEEEE | 18 |
| domain | GGCEL | |
| human DAP10 costimulatory | LCARPRRSPAQEDGKVYINMPGRG | 19 |
| domain | | |
| human DAP12 costimulatory | YFLGRLVPRGRGAAEAATRKQRITETESPYQELQGQR | 20 |
| domain | SDVYSDLNTQRPYYK | |
| human 2B4 costimulatory | WRRKRKEKQSETSPKEFLTIYEDVKDLKTRRNHEQEQ | 21 |
| domain | TFPGGGSTIYSMIQSQSSAPTSQEPAYTLYSLIQPSR | |
| | KSGSRKRNHSPSFNSTIYEVIGKSQPKAQNPARLSRK | |
| | ELENFDVYS | |
| human OX40 costimulatory | ALYLLRRDQRLPPDAHKPPGGGSFRTPIQEEQADAHS | 22 |
| domain | TLAKI | |
| human CD27 costimulatory | HQRRKYRSNKGESPVEPAEPCHYSCPREEEGSTIPIQ | 23 |
| domain | EDYRKPEPACSP | |
| human CD27 costimulatory | QRRKYRSNKGESPVEPAEPCHYSCPREEEGSTIPIQE | 24 |
| domain | DYRKPEPACSP | |

ACTIVATION DOMAINS

| | | |
|---|---|---|
| human CD3zeta intracellular | DIRVKFSRSADAPAYQQGQNQLYNELNLGRREEYDVL | 25 |
| signaling domain | DKRRGRDPEMGGKPRRKNPQEGLYNELQKDKMAEAYS | |
| | EIGMKGERRRGKGHDGLYQGLSTATKDTYDALHMQAL | |
| | PPR | |
| human CD3zeta intracellular | RVKFSRSADAPAYQQGQNQLYNELNLGRREEYDVLDK | 26 |
| signaling domain | RRGRDPEMGGKPRRKNPQEGLYNELQKDKMAEAYSEI | |
| | GMKGERRRGKGHDGLYQGLSTATKDTYDALHMQALPP | |
| | R | |
| CD3ε intracellular signaling | KNRKAKAKPVTRGAGAGGRQRGQNKERPPPVPNPDYE | 27 |
| domain | PIRKGQRDLYSGLNQRRI | |
| CD3ε intracellular signaling | KNRKAKAKPVTRGAGAGGRQRGQNKERPPPVPNPDYE | 28 |
| domain alternate sequence | PIRKGQRDLYSGL | |

Table 3 provides exemplary nucleic acid sequences of the domains which can be used to encode the CARs described herein. In some embodiments, a nucleic acid sequence encoding a CAR provided herein comprises one or more sequences described in Table 9, or a fragment or portion thereof.

TABLE 3
Exemplary Nucleic Acid Sequences of CAR Domains

| | | |
|---|---|---|
| | | SEQ ID |
| Exemplary CAR domains | Nucleic Acid Sequence | NO: |

SIGNAL PEPTIDE

| | | |
|---|---|---|
| human CD8alpha signal | GCTAGCGCCACCATGGCTCTGCCTGTGACAGCTCTGC | 29 |
| sequence | TGCTGCCTCTGGCCCTGCTGCTCCATGCTGCTAGACC | |
| | T | |
| human CD8alpha signal | ATGGCTCTGCCTGTGACAGCTCTGCTGCTGCCTCTGG | 30 |
| sequence | CCCTGCTGCTCCATGCTGCTAGACCT | |
| human IgG heavy chain signal | GGATCCATGGAGTTTGGCCTGAGCTGGCTGTTCCTGG | 31 |
| sequence | TGGCCATCCTCAAGGGCGTGCAGTGCTCCAGG | |
| human IgG heavy chain signal | ATGGAGTTTGGCCTGAGCTGGCTGTTCCTGGTGGCCA | 32 |
| sequence | TCCTCAAGGGCGTGCAGTGCTCCAGG | |

HINGES

| | | |
|---|---|---|
| human CD8alpha hinge | CTCGAGACCACCACCCCCGCCCCTAGGCCTCCCACAC | 33 |
| domain | CTGCCCCCACAATCGCCTCCCAGCCTCTCAGCCTGAG | |
| | GCCTGAAGCTTGCAGGCCCGCTGCCGGAGGAGCTGTC | |
| | CATACCAGGGGACTCGACTTCGCCTGCGAC | |
| human CD8alpha hinge | ACCACCACCCCCGCCCCTAGGCCTCCCACACCTGCCC | 34 |
| domain | CCACAATCGCCTCCCAGCCTCTCAGCCTGAGGCCTGA | |
| | AGCTTGCAGGCCCGCTGCCGGAGGAGCTGTCCATACC | |
| | AGGGGACTCGACTTCGCCTGCGAC | |
| human CD28 hinge domain | TCTAGAATCGAAGTGATGTACCCTCCACCTTACCTGG | 35 |
| | ACAACGAGAAGTCCAACGGCACCATCATCCACGTGAA | |
| | GGGCAAGCACCTGTGTCCTTCTCCACTGTTCCCCGGA | |
| | CCTAGCAAGCCT | |
| human CD28 hinge domain | ATCGAAGTGATGTACCCTCCACCTTACCTGGACAACG | 36 |
| | AGAAGTCCAACGGCACCATCATCCACGTGAAGGGCAA | |
| | GCACCTGTGTCCTTCTCCACTGTTCCCCGGACCTAGC | |
| | AAGCCT | |
| human IgG1 hinge domain | GAGCCCAAGAGCTGCGACAAGACCCACACCTGCCCCC | 37 |
| | CCTGCCCCGCCCCCGAGCTGCTGGGCGGCCCCAGCGT | |
| | GTTCCTGTTCCCCCCCAAGCCCAAGGACACCCTGATG | |
| | ATCAGCCGGACCCCCGAGGTGACCTGCGTGGTGGTGG | |
| | ACGTGAGCCACGAGGACCCCGAGGTGAAGTTCAACTG | |
| | GTACGTGGACGGCGTGGAGGTGCACAACGCCAAGACC | |
| | AAGCCCCGGGAGGAGCAGTACAACAGCACCTACCGGG | |
| | TGGTGAGCGTGCTGACCGTGCTGCACCAGGACTGGCT | |
| | GAACGGCAAGGAGTACAAGTGCAAGGTGAGCAACAAG | |
| | GCCCTGCCCGCCCCCATCGAGAAGACCATCAGCAAGG | |
| | CCAAGGGCCAGCCCCGGGAGCCCCAGGTGTACACCCT | |
| | GCCCCCCAGCCGGGACGAGCTGACCAAGAACCAGGTG | |
| | AGCCTGACCTGCCTGGTGAAGGGCTTCTACCCCAGCG | |
| | ACATCGCCGTGGAGTGGGAGAGCAACGGCCAGCCCGA | |
| | GAACAACTACAAGACCACCCCCCCCGTGCTGGACAGC | |
| | GACGGCAGCTTCTTCCTGTACAGCAAGCTGACCGTGG | |
| | ACAAGAGCCGGTGGCAGCAGGGCAACGTGTTCAGCTG | |
| | CAGCGTGATGCACGAGGCCCTGCACAACCACTACACC | |
| | CAGAAGAGCCTGAGCCTGAGCCCCGGCAAG | |
| Human IgG1 hinge domain | GAGCCCAAGAGCTGCGACAAGACCCACACCTGCCCC | 38 |
| human IgG4 hinge domain | GAGAGCAAGTACGGCCCCCCCTGCCCCAGCTGCCCCG | 39 |
| | CCCCCGAGTTCCTGGGCGGCCCCAGCGTGTTCCTGTT | |
| | CCCCCCCAAGCCCAAGGACACCCTGATGATCAGCCGG | |
| | ACCCCCGAGGTGACCTGCGTGGTGGTGGACGTGAGCC | |
| | AGGAGGACCCCGAGGTGCAGTTCAACTGGTACGTGGA | |
| | CGGCGTGGAGGTGCACAACGCCAAGACCAAGCCCCGG | |
| | GAGGAGCAGTTCAACAGCACCTACCGGGTGGTGAGCG | |
| | TGCTGACCGTGCTGCACCAGGACTGGCTGAACGGCAA | |
| | GGAGTACAAGTGCAAGGTGAGCAACAAGGGCCTGCCC | |
| | AGCAGCATCGAGAAGACCATCAGCAAGGCCAAGGGCC | |
| | AGCCCCGGGAGCCCCAGGTGTACACCCTGCCCCCCAG | |
| | CCAGGAGGAGATGACCAAGAACCAGGTGAGCCTGACC | |
| | TGCCTGGTGAAGGGCTTCTACCCCAGCGACATCGCCG | |
| | TGGAGTGGGAGAGCAACGGCCAGCCCGAGAACAACTA | |
| | CAAGACCACCCCCCCCGTGCTGGACAGCGACGGCAGC | |
| | TTCTTCCTGTACAGCCGGCTGACCGTGGACAAGAGCC | |
| | GGTGGCAGGAGGGCAACGTGTTCAGCTGCAGCGTGAT | |
| | GCACGAGGCCCTGCACAACCACTACACCCAGAAGAGC | |
| | CTGAGCCTGAGCCTGGGCAAG | |
| human IgG4 hinge domain | GAGAGCAAGTACGGCCCCCCCTGCCCC | 40 |

TRANSMEMBRANE

| | | |
|---|---|---|
| human CD8alpha | ATTTACATTTGGGCCCCTCTGGCTGGAACCTGCGGAG | 41 |
| transmembrane domain | TCCTGCTGCTGTCCCTGGTGATCACACTGTACTGT | |
| human CD28 transmembrane | TTCTGGGTGCTCGTTGTTGTTGGCGGCGTGCTGGCCT | 42 |
| domain | GTTACAGCCTGCTGGTTACCGTGGCCTTCATCATCTT | |
| | TTGGGTG | |
| human CD3ε transmembrane | GTGATGAGCGTGGCCACCATCGTGATCGTGGACATCT | 43 |
| domain | GTATCACCGGCGGACTGCTGCTGCTGGTGTACTACTG | |
| | GTCC | |
| human 2B4 transmembrane | TTCCTGGTCATCATCGTGATCCTGAGCGCCCTGTTCC | 92 |
| domain | TGGGCACCCTGGCCTGTTTCTGCGTG | |

COSTIMULATORY DOMAINS

| | | |
|---|---|---|
| human CD28 costimulatory | CGAAGCAAGCGGAGCCGGGGAGGACACAGCGACTACA | 44 |
| domain | TGAACATGACCCCTCGGAGGCCAGGCCCCACCAGAAA | |
| | GCACTACCAGCCCTACGCCCCTCCCCGGGACTTTGCC | |
| | GCCTATCGGAGC | |
| human CD28 costimulatory | CGAAGCAAGCGGAGCCGGCTGCTGCACAGCGACTACA | 45 |
| domain | TGAACATGACCCCTCGGAGGCCAGGCCCCACCAGAAA | |
| | GCACTACCAGCCCTACGCCCCTCCCCGGGACTTTGCC | |
| | GCCTATCGGAGC | |
| human 4-1BB costimulatory | AAGAGGGGCAGAAAGAAGCTGCTCTACATCTTCAAGC | 46 |
| domain | AGCCCTTTATGAGACCCGTGCAGACAACCCAGGAGGA | |
| | AGACGGATGCAGCTGCAGGTTCCCTGAGGAGGAGGAG | |
| | GGCGGCTGCGAACTG | |
| human DAP10 costimulatory | CTGTGCGCCCGGCCCCGGCGGAGCCCCGCCCAGGAGG | 47 |
| domain | ACGGCAAGGTGTACATCAACATGCCCGGCCGGGGC | |
| human DAP12 costimulatory | TACTTCCTGGGCCGGCTGGTGCCCCGGGGCCGGGGCG | 48 |
| domain | CCGCCGAGGCCGCCACCCGGAAGCAGCGGATCACCGA | |
| | GACCGAGAGCCCCTACCAGGAGCTGCAGGGCCAGCGG | |
| | AGCGACGTGTACAGCGACCTGAACACCCAGCGGCCCT | |
| | ACTACAAG | |
| human 2B4 costimulatory | TGGCGGCGGAAGCGGAAGGAGAAGCAGAGCGAGACCA | 49 |
| domain | GCCCCAAGGAGTTCCTGACCATCTACGAGGACGTGAA | |
| | GGACCTGAAGACCCGGCGGAACCACGAGCAGGAGCAG | |
| | ACCTTCCCCGGGGGGGGCAGCACCATCTACAGCATGA | |
| | TCCAGAGCCAGAGCAGCGCCCCCACCAGCCAGGAGCC | |
| | CGCCTACACCCTGTACAGCCTGATCCAGCCCAGCCGG | |
| | AAGAGCGGCAGCCGGAAGCGGAACCACAGCCCCAGCT | |
| | TCAACAGCACCATCTACGAGGTGATCGGCAAGAGCCA | |
| | GCCCAAGGCCCAGAACCCCGCCCGGCTGAGCCGGAAG | |
| | GAGCTGGAGAACTTCGACGTGTACAGC | |
| human OX40 costimulatory | GCCCTGTACCTGCTGCGGCGGGACCAGCGGCTGCCCC | 50 |
| domain | CCGACGCCCACAAGCCCCCCGGCGGCGGCAGCTTCCG | |
| | GACCCCCATCCAGGAGGAGCAGGCCGACGCCCACAGC | |
| | ACCCTGGCCAAGATC | |
| human CD27 costimulatory | CACCAGCGGCGGAAGTACCGGAGCAACAAGGGCGAGA | 51 |
| domain | GCCCCGTGGAGCCCGCCGAGCCCTGCCACTACAGCTG | |
| | CCCCCGGGAGGAGGAGGGCAGCACCATCCCCATCCAG | |
| | GAGGACTACCGGAAGCCCGAGCCCGCCTGCAGCCCC | |
| human CD27 costimulatory | CAGCGGCGGAAGTACCGGAGCAACAAGGGCGAGAGCC | 52 |
| domain | CCGTGGAGCCCGCCGAGCCCTGCCACTACAGCTGCCC | |
| | CCGGGAGGAGGAGGGCAGCACCATCCCCATCCAGGAG | |
| | GACTACCGGAAGCCCGAGCCCGCCTGCAGCCCC | |

ACTIVATION DOMAINS

| | | |
|---|---|---|
| human CD3zeta intracellular | GATATCAGGGTGAAGTTCAGCAGGAGCGCCGACGCCC | 53 |
| signaling domain | CCGCTTATCAACAGGGCCAGAACCAGCTGTACAACGA | |
| | GCTGAACCTCGGCAGAAGAGAGGAGTATGACGTGCTG | |
| | GACAAGAGGAGGGGCAGGGACCCTGAGATGGGCGGCA | |
| | AGCCTAGAAGAAAGAACCCCCAGGAAGGCCTCTACAA | |
| | CGAACTGCAGAAGGACAAGATGGCCGAGGCCTACAGC | |
| | GAGATCGGCATGAAAGGCGAGAGAAGGAGGGGAAAGG | |
| | GACATGACGGCCTGTACCAGGGACTCTCCACAGCCAC | |
| | CAAGGACACCTACGATGCCCTGCACATGCAGGCTCTG | |
| | CCCCCTAGA | |
| human CD3zeta intracellular | AGGGTGAAGTTCAGCAGGAGCGCCGACGCCCCCGCTT | 54 |
| signaling domain | ATCAACAGGGCCAGAACCAGCTGTACAACGAGCTGAA | |
| | CCTCGGCAGAAGAGAGGAGTATGACGTGCTGGACAAG | |
| | AGGAGGGGCAGGGACCCTGAGATGGGGGGCAAGCCTA | |
| | GAAGAAAGAACCCCCAGGAAGGCCTCTACAACGAACT | |
| | GCAGAAGGACAAGATGGCCGAGGCCTACAGCGAGATC | |
| | GGCATGAAAGGCGAGAGAAGGAGGGGAAAGGGACATG | |
| | ACGGCCTGTACCAGGGACTCTCCACAGCCACCAAGGA | |
| | CACCTACGATGCCCTGCACATGCAGGCTCTGCCCCCT | |
| | AGA | |
| CD3ε intracellular signaling | AAGAACCGGAAGGCCAAGGCCAAGCCAGTGACAAGAG | 55 |
| domain alternate sequence | GTGCTGGTGCTGGCGGCAGACAGAGAGGCCAGAACAA | |
| | AGAAAGACCTCCTCCTGTGCCTAATCCTGACTACGAG | |
| | CCCATCCGGAAGGGCCAGAGAGATCTGTATAGCGGCC | |
| | TC | |
| CD3ε intracellular signaling | AAGAACCGGAAGGCCAAGGCCAAGCCAGTGACAAGAG | 56 |
| domain | GTGCTGGTGCTGGCGGCAGACAGAGAGGCCAGAACAA | |
| | AGAAAGACCTCCTCCTGTGCCTAATCCTGACTACGAG | |
| | CCCATCCGGAAGGGCCAGAGAGATCTGTATAGCGGCC | |
| | TCAACCAGAGAAGAATC | |

### G. Exemplary CAR Constructs

### i) Anti-CD22 CAR Constructs

Disclosed herein are CARs that specifically bind to CD22. In some embodiments, the CAR (e.g. first CAR) comprises an antigen recognition domain that specifically binds human CD22, a hinge domain comprising or consisting of a CD8α hinge domain, a transmembrane domain comprising or consisting of a CD8α transmembrane domain, a costimulatory domain comprising or consisting of a 4-1BB costimulatory domain; and an intracellular signaling domain comprising or consisting of a CD3zeta activation domain. Also disclosed herein are nucleic acid sequences encoding said CARs. In some embodiments, a T cell or population of T cells described herein is genetically modified to express at least one of the exemplary anti-CD22 CAR constructs described herein.

### Exemplary Anti-CD22-CD3z CARs (First CARs)

An exemplary anti-CD22 CAR, also referred to as “2G-22BBz CAR”, “CAR1”, “CAR1-1”, “CD22 CAR”, “2nd generation CAR”, “2G CAR”, or “2G-22BBz” amino acid sequence is shown below. (CD8α signal peptide, CD22 scFv (m971), CD8α hinge, CD8α transmembrane domain, 4-BB signaling domain, CD3z signaling domain)

(SEQ ID NO: 57)
ASATMALPVTALLLPLALLLHAARPQVQLQQSGPGLVKPSQTLSLTCAI
SGDSVSSNSAAWNWIRQSPSRGLEWLGRTYYRSKWYNDYAVSVKSRITI
NPDTSKNQFSLQLNSVTPEDTAVYYCAREVTGDLEDAFDIWGQGTMVTV
SSGGGGSDIQMTQSPSSLSASVGDRVTITCRASQTIWSYLNWYQQRPGK
APNLLIYAASSLQSGVPSRFSGRGSGTDFTLTISSLQAEDFATYYCQQS
YSIPQTFGQGTKLEIKLETTTPAPRPPTPAPTIASQPLSLRPEACRPAA
GGAVHTRGLDFACDIYIWAPLAGTCGVLLLSLVITLYCKRGRKKLLYIF
KQPFMRPVQTTQEEDGCSCRFPEEEEGGCELDIRVKFSRSADAPAYQQG
QNQLYNELNLGRREEYDVLDKRRGRDPEMGGKPRRKNPQEGLYNELQKD
KMAEAYSEIGMKGERRRGKGHDGLYQGLSTATKDTYDALHMQALPPR

In some embodiments, the anti-CD22 CAR provided herein may comprise or consist of an amino acid sequence having at least 90, 91%, 92%, 93%, 94%, 95%, 96%, 97%, 98%, 99% or 100% identity with the amino acid sequence of SEQ ID NO: 57.

An exemplary anti-CD22 CAR, “CAR1”, “CAR1-2”, “CD22 CAR”, “2nd generation CAR”, “2G CAR”, or “2G-22BBz” amino acid sequence is shown below. (CD8α signal peptide, CD22 scFv (m971), CD8α hinge, CD8α transmembrane domain. 4-1BB signaling domain, (CD3z signaling domain)

(SEQ ID NO: 86)
MALPVTALLLPLALLLHAARPQVQLQQSGPGLVKPSQTLSLTCAISGDS
VSSNSAAWNWIRQSPSRGLEWLGRTYYRSKWYNDYAVSVKSRITINPDT
SKNQFSLQLNSVTPEDTAVYYCAREVTGDLEDAFDIWGQGTMVTVSSGG
GGSDIQMTQSPSSLSASVGDRVTITCRASQTIWSYLNWYQQRPGKAPNL
LIYAASSLQSGVPSRFSGRGSGTDFTLTISSLQAEDFATYYCQQSYSIP
QTFGQGTKLEIKLETTTPAPRPPTPAPTIASQPLSLRPEACRPAAGGAV
HTRGLDFACDIYIWAPLAGTCGVLLLSLVITLYCKRGRKKLLYIFKQPF
MRPVQTTQEEDGCSCRFPEEEEGGCELDIRVKFSRSADAPAYQQGQNQL
YNELNLGRREEYDVLDKRRGRDPEMGGKPRRKNPQEGLYNELQKDKMAE
AYSEIGMKGERRRGKGHDGLYQGLSTATKDTYDALHMQALPPR

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO 86.

An exemplary anti-CD22 CAR, “CAR1”, “CAR1-3”, “CD22 CAR”, “2nd generation CAR”, “2G CAR”, or “2G-22BBz” amino acid sequence is shown below. (CD8α signal peptide, high affinity CD22 scFv (m971), CD8α hinge, CD8α transmembrane domain. 4-BB signaling domain, CD3z signaling domain).

(SEQ ID NO: 98)
MALPVTALLLPLALLLHAARPQVQLQQSGPGMVKPSQTLSLTCAISGDS
VSSNSVAWNWIRQSPSRGLEWLGRTYYRSTWYNDYAVSMKSRITINPDT
NKNQFSLQLNSVTPEDTAVYYCAREVTGDLEDAFDIWGQGTMVTVSSGG
GGSGGGGSGGGGSDIQMIQSPSSLSASVGDRVTITCRASQTIWSYLNWY
RQRPGEAPNLLIYAASSLQSGVPSRFSGRGSGTDFTLTISSLQAEDFAT
YYCQQSYSIPQTFGQGTKLEIKLETTTPAPRPPTPAPTIASQPLSLRPE
ACRPAAGGAVHTRGLDFACDIYIWAPLAGTCGVLLLSLVITLYCKRGRK
KLLYIFKQPFMRPVQTTQEEDGCSCRFPEEEEGGCELDIRVKFSRSADA
PAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQA
LPPR

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO. 98.

An exemplary anti-CD22 CAR, “CAR1”, “CAR1-4”, “CD22 CAR”, “2nd generation CAR”, “2G CAR”, or “2G-22BBz” amino acid sequence is shown below. (CD8α signal peptide. His-tag, high affinity CD22 scFv (m971), CD8α hinge, CD8α transmembrane domain, 4-4BB signaling domain, CD3z signaling domain).

(SEQ ID NO: 100)
MALPVTALLLPLALLLHAARPHHHHHHQVQLQQSGPGMVKPSQTLSLTC
AISGDSVSSNSVAWNWIRQSPSRGLEWLGRTYYRSTWYNDYAVSMKSRI
TINPDTNKNQFSLQLNSVTPEDTAVYYCAREVTGDLEDAFDIWGQGTMV
TVSSGGGGSGGGGSGGGGSDIQMIQSPSSLSASVGDRVTITCRASQTIW
SYLNWYRQRPGEAPNLLIYAASSLQSGVPSRFSGRGSGTDFTLTISSLQ
AEDFATYYCQQSYSIPQTFGQGTKLEIKLETTTPAPRPPTPAPTIASQP
LSLRPEACRPAAGGAVHTRGLDFACDIYIWAPLAGTCGVLLLSLVITLY
CKRGRKKLLYIFKQPFMRPVQTTQEEDGCSCRFPEEEEGGCELDIRVKF
SRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYD
ALHMQALPPR

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO. 100.

### Exemplary Anti-CD22-CD3z CAR Polynucleotide Sequences (First CARs)

An exemplary anti-CD22 CAR, “CAR.”, “CD22 CAR”, “2nd generation CAR”, “2G CAR”, or “2G-22BBz” polynucleotide sequence is shown below. (CD8α signal peptide, CD22 scFv (m971), CD8α hinge, CD8α transmembrane domain. 4-1BB signaling domain, CD3z signaling domain)

(SEQ ID NO: 58)
GCTAGCGCCACCATGGCTCTGCCTGTGACAGCTCTGCTGCTGCCTCTGGC
CCTGCTGCTCCATGCTGCTAGACCTCAGGTGCAGCTCCAGCAGTCTGGCC
CAGGACTGGTCAAGCCTAGCCAGACCCTGAGCCTGACCTGCGCCATCAGC
GGCGACAGCGTGTCCTCTAACAGCGCCGCCTGGAACTGGATCAGACAGAG
CCCCAGCAGAGGCCTGGAATGGCTGGGCCGGACCTACTACCGGTCCAAGT
GGTACAACGACTACGCCGTGTCCGTGAAGTCCCGGATCACCATCAACCCC
GACACCAGCAAGAACCAGTTCTCCCTGCAGCTGAACAGCGTGACCCCTGA
GGACACCGCCGTGTACTACTGCGCCAGAGAAGTGACCGGCGACCTGGAAG
ATGCCTTCGACATCTGGGGCCAGGGCACCATGGTCACCGTGTCTAGCGGA
GGCGGCGGAAGCGACATCCAGATGACCCAGAGCCCTAGCTCCCTGAGCGC
CAGCGTGGGCGACAGAGTGACCATCACCTGTCGGGCCAGCCAGACCATCT
GGTCCTACCTGAATTGGTATCAGCAGCGGCCAGGCAAGGCCCCTAACCTG
CTGATCTATGCCGCCAGCAGCCTGCAGAGCGGCGTGCCAAGCAGATTCTC
TGGCAGAGGCTCCGGCACCGACTTCACCCTGACAATCAGTTCCCTGCAGG
CCGAGGACTTCGCCACCTACTACTGCCAGCAGTCCTACAGCATCCCTCAG
ACCTTCGGCCAGGGGACCAAGCTGGAAATCAAGCTCGAGACCACCACCCC
CGCCCCTAGGCCTCCCACACCTGCCCCCACAATCGCCTCCCAGCCTCTCA
GCCTGAGGCCTGAAGCTTGCAGGCCCGCTGCCGGAGGAGCTGTCCATACC
AGGGGACTCGACTTCGCCTGCGACATTTACATTTGGGCCCCTCTGGCTGG
AACCTGCGGAGTCCTGCTGCTGTCCCTGGTGATCACACTGTACTGTAAGA
GGGGCAGAAAGAAGCTGCTCTACATCTTCAAGCAGCCCTTTATGAGACCC
GTGCAGACAACCCAGGAGGAAGACGGATGCAGCTGCAGGTTCCCTGAGGA
GGAGGAGGGCGGCTGCGAACTGGATATCAGGGTGAAGTTCAGCAGGAGCG
CCGACGCCCCCGCTTATCAACAGGGCCAGAACCAGCTGTACAACGAGCTG
AACCTCGGCAGAAGAGAGGAGTATGACGTGCTGGACAAGAGGAGGGGCAG
GGACCCTGAGATGGGCGGCAAGCCTAGAAGAAAGAACCCCCAGGAAGGCC
TCTACAACGAACTGCAGAAGGACAAGATGGCCGAGGCCTACAGCGAGATC
GGCATGAAAGGCGAGAGAAGGAGGGGAAAGGGACATGACGGCCTGTACCA
GGGACTCTCCACAGCCACCAAGGACACCTACGATGCCCTGCACATGCAGG
CTCTGCCCCCTAGA

In some embodiments, the anti-CD22 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 58.

An exemplary anti-CD22 CAR, “CAR1”, “CD22 CAR”, “2nd generation CAR”, “2G CAR”, or “2G-22BBz” polynucleotide sequence is shown below (CD8α signal peptide. CD22 scFv (m971), CD8α hinge, CD8α transmembrane domain. 4-4BB signaling domain, CD3:

(SEQ ID NO: 87)
ATGGCTCTGCCTGTGACAGCTCTGCTGCTGCCTCTGGCCCTGCTGCTCCA
TGCTGCTAGACCTCAGGTGCAGCTCCAGCAGTCTGGCCCAGGACTGGTCA
AGCCTAGCCAGACCCTGAGCCTGACCTGCGCCATCAGCGGCGACAGCGTG
TCCTCTAACAGCGCCGCCTGGAACTGGATCAGACAGAGCCCCAGCAGAGG
CCTGGAATGGCTGGGCCGGACCTACTACCGGTCCAAGTGGTACAACGACT
ACGCCGTGTCCGTGAAGTCCCGGATCACCATCAACCCCGACACCAGCAAG
AACCAGTTCTCCCTGCAGCTGAACAGCGTGACCCCTGAGGACACCGCCGT
GTACTACTGCGCCAGAGAAGTGACCGGCGACCTGGAAGATGCCTTCGACA
TCTGGGGCCAGGGCACCATGGTCACCGTGTCTAGCGGAGGCGGCGGAAGC
GACATCCAGATGACCCAGAGCCCTAGCTCCCTGAGCGCCAGCGTGGGCGA
CAGAGTGACCATCACCTGTCGGGCCAGCCAGACCATCTGGTCCTACCTGA
ATTGGTATCAGCAGCGGCCAGGCAAGGCCCCTAACCTGCTGATCTATGCC
GCCAGCAGCCTGCAGAGCGGCGTGCCAAGCAGATTCTCTGGCAGAGGCTC
CGGCACCGACTTCACCCTGACAATCAGTTCCCTGCAGGCCGAGGACTTCG
CCACCTACTACTGCCAGCAGTCCTACAGCATCCCTCAGACCTTCGGCCAG
GGGACCAAGCTGGAAATCAAGCTCGAGACCACCACCCCCGCCCCTAGGCC
TCCCACACCTGCCCCCACAATCGCCTCCCAGCCTCTCAGCCTGAGGCCTG
AAGCTTGCAGGCCCGCTGCCGGAGGAGCTGTCCATACCAGGGGACTCGAC
TTCGCCTGCGACATTTACATTTGGGCCCCTCTGGCTGGAACCTGCGGAGT
CCTGCTGCTGTCCCTGGTGATCACACTGTACTGTAAGAGGGGCAGAAAGA
AGCTGCTCTACATCTTCAAGCAGCCCTTTATGAGACCCGTGCAGACAACC
CAGGAGGAAGACGGATGCAGCTGCAGGTTCCCTGAGGAGGAGGAGGGCGG
CTGCGAACTGGATATCAGGGTGAAGTTCAGCAGGAGCGCCGACGCCCCCG
CTTATCAACAGGGCCAGAACCAGCTGTACAACGAGCTGAACCTCGGCAGA
AGAGAGGAGTATGACGTGCTGGACAAGAGGAGGGGCAGGGACCCTGAGAT
GGGCGGCAAGCCTAGAAGAAAGAACCCCCAGGAAGGCCTCTACAACGAAC
TGCAGAAGGACAAGATGGCCGAGGCCTACAGCGAGATCGGCATGAAAGGC
GAGAGAAGGAGGGGAAAGGGACATGACGGCCTGTACCAGGGACTCTCCAC
AGCCACCAAGGACACCTACGATGCCCTGCACATGCAGGCTCTGCCCCCTA
GA

In some embodiments, the anti-CD22 CAR provided herein is encoded by a polynucleotide sequence comprising or consisting of an nucleic acid sequence having at least 90/o, 91%, 92%, 93%, 94%, 95%, 96%, 97%, 98%, 99% or 100% identity with the nucleic acid sequence of SEQ ID NO: 87

An exemplary anti-CD22 CAR, “CAR1”, “CAR1-3”, “CD22 CAR”, “2nd generation CAR”, “2G CAR”, or “2G-22BBz” polynucleotide sequence is shown below.

(SEQ ID NO: 99)
ATGGCACTGCCAGTGACTGCATTACTCTTGCCACTCGCGCTACTGTTACA
CGCAGCACGTCCACAAGTCCAATTGCAACAAAGCGGGCCGGGCATGGTGA
AACCGAGTCAAACGTTATCTCTTACGTGTGCGATTTCGGGGGATAGTGTC
AGCAGCAATTCAGTGGCGTGGAATTGGATTCGCCAATCGCCGAGTCGCGG
GTTGGAGTGGCTCGGGCGCACGTATTATCGCAGCACATGGTATAATGATT
ATGCGGTCAGCATGAAAAGCCGCATTACGATTAATCCGGATACGAACAAA
AATCAATTTAGCTTACAATTAAATTCCGTCACGCCGGAAGATACAGCGGT
CTATTATTGTGCGCGCGAGGTCACGGGGGATCTCGAAGACGCGTTTGATA
TTTGGGGGCAAGGGACCATGGTGACTGTCAGCTCTGGTGGAGGGGGCAGT
GGAGGTGGGGGATCGGGAGGTGGTGGCAGTGATATTCAAATGATCCAAAG
TCCATCCAGCCTATCCGCATCTGTCGGAGATCGCGTAACGATTACGTGCC
GCGCGAGTCAAACGATTTGGAGCTATCTGAACTGGTACCGGCAACGCCCG
GGCGAAGCGCCGAATCTCTTGATTTACGCGGCGTCCTCATTACAGTCGGG
TGTCCCGAGCCGCTTTAGCGGCCGCGGAAGCGGTACGGATTTTACGTTAA
CCATTAGCAGCCTCCAGGCGGAAGATTTTGCGACGTATTACTGTCAACAG
AGCTATAGCATTCCGCAGACGTTTGGTCAGGGCACGAAATTGGAGATTAA
ACTCGAGACCACCACCCCCGCCCCTAGGCCTCCCACACCTGCCCCCACAA
TCGCCTCCCAGCCTCTCAGCCTGAGGCCTGAAGCTTGCAGGCCCGCTGCC
GGAGGAGCTGTCCATACCAGGGGACTCGACTTCGCCTGCGACATTTACAT
TTGGGCCCCTCTGGCTGGAACCTGCGGAGTCCTGCTGCTGTCCCTGGTGA
TCACACTGTACTGTAAGAGGGGCAGAAAGAAGCTGCTCTACATCTTCAAG
CAGCCCTTTATGAGACCCGTGCAGACAACCCAGGAGGAAGACGGATGCAG
CTGCAGGTTCCCTGAGGAGGAGGAGGGCGGCTGCGAACTGGATATCAGGG
TGAAGTTCAGCAGGAGCGCCGACGCCCCCGCTTATCAACAGGGCCAGAAC
CAGCTGTACAACGAGCTGAACCTCGGCAGAAGAGAGGAGTATGACGTGCT
GGACAAGAGGAGGGGCAGGGACCCTGAGATGGGCGGCAAGCCTAGAAGAA
AGAACCCCCAGGAAGGCCTCTACAACGAACTGCAGAAGGACAAGATGGCC
GAGGCCTACAGCGAGATCGGCATGAAAGGCGAGAGAAGGAGGGGAAAGGG
ACATGACGGCCTGTACCAGGGACTCTCCACAGCCACCAAGGACACCTACG
ATGCCCTGCACATGCAGGCTCTGCCCCCTAGA

In some embodiments, the anti-CD22 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO. 99.

An exemplary anti-CD22 CAR, “CAR1”, “CAR1-4”, “CD22 CAR”, “2nd generation CAR”, “2G CAR”, or “2G-22BBZ” polynucleotide sequence is shown below.

(SEQ ID NO: 101)
ATGGCACTGCCAGTGACTGCATTACTCTTGCCACTCGCGCTACTGTTACA
CGCAGCACGTCCACATCACCATCACCATCACCAAGTCCAATTGCAACAAA
GCGGGCCGGGCATGGTGAAACCGAGTCAAACGTTATCTCTTACGTGTGCG
ATTTCGGGGGATAGTGTCAGCAGCAATTCAGTGGCGTGGAATTGGATTCG
CCAATCGCCGAGTCGCGGGTTGGAGTGGCTCGGGCGCACGTATTATCGCA
GCACATGGTATAATGATTATGCGGTCAGCATGAAAAGCCGCATTACGATT
AATCCGGATACGAACAAAAATCAATTTAGCITACAATTAAATTCCGTCAC
GCCGGAAGATACAGCGGTCTATTATTGTGCGCGCGAGGTCACGGGGGATC
TCGAAGACGCGTTTGATATTTGGGGGCAAGGGACCATGGTGACTGTCAGC
TCTGGTGGAGGGGGCAGTGGAGGTGGGGGATCGGGAGGTGGTGGCAGTGA
TATTCAAATGATCCAAAGTCCATCCAGCCTATCCGCATCTGTCGGAGATC
GCGTAACGATTACGTGCCGCGCGAGTCAAACGATTTGGAGCTATCTGAAC
TGGTACCGGCAACGCCCGGGCGAAGCGCCGAATCTCTTGATTTACGCGGC
GTCCTCATTACAGTCGGGTGTCCCGAGCCGCTTTAGCGGCCGCGGAAGCG
GTACGGATTTTACGTTAACCATTAGCAGCCTCCAGGCGGAAGATTTTGCG
ACGTATTACTGTCAACAGAGCTATAGCATTCCGCAGACGTTTGGTCAGGG
CACGAAATTGGAGATTAAACTCGAGACCACCACCCCCGCCCCTAGGCCTC
CCACACCTGCCCCCACAATCGCCTCCCAGCCTCTCAGCCTGAGGCCTGAA
GCTTGCAGGCCCGCTGCCGGAGGAGCTGTCCATACCAGGGGACTCGACTT
CGCCTGCGACATTTACATTTGGGCCCCTCTGGCTGGAACCTGCGGAGTCC
TGCTGCTGTCCCTGGTGATCACACTGTACTGTAAGAGGGGCAGAAAGAAG
CTGCTCTACATCTTCAAGCAGCCCTTTATGAGACCCGTGCAGACAACCCA
GGAGGAAGACGGATGCAGCTGCAGGTTCCCTGAGGAGGAGGAGGGCGGCT
GCGAACTGGATATCAGGGTGAAGTTCAGCAGGAGCGCCGACGCCCCCGCT
TATCAACAGGGCCAGAACCAGCTGTACAACGAGCTGAACCTCGGCAGAAG
AGAGGAGTATGACGTGCTGGACAAGAGGAGGGGCAGGGACCCTGAGATGG
GCGGCAAGCCTAGAAGAAAGAACCCCCAGGAAGGCCTCTACAACGAACTG
CAGAAGGACAAGATGGCCGAGGCCTACAGCGAGATCGGCATGAAAGGCGA
GAGAAGGAGGGGAAAGGGACATGACGGCCTGTACCAGGGACTCTCCACAG
CCACCAAGGACACCTACGATGCCCTGCACATGCAGGCTCTGCCCCCTAGA
AG

In some embodiments, the anti-CD22 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO. 101.

An exemplary anti-CD22 CAR “CAR2”, “CAR2-2” or “CD22 CAR” polynucleotide sequence is shown below.

(SEQ ID NO: 97)
ATGGCTCTGCCTGTGACAGCTCTGCTGCTGCCTCTGGCCCTGCTGCTCCA
TGCTGCTAGACCTCAGGTGCAGCTCCAGCAGTCTGGCCCAGGAATGGTCA
AGCCTAGCCAGACCCTGAGCCTGACCTGCGCCATCAGCGGCGACAGCGTG
TCCTCTAACAGCGTCGCCTGGAACTGGATCAGACAGAGCCCCAGCAGAGG
CCTGGAATGGCTGGGCCGGACCTACTACCGGTCCACGTGGTACAACGACT
ACGCCGTGTCCATGAAGTCCCGGATCACCATCAACCCCGACACCAACAAG
AACCAGTTCTCCCTGCAGCTGAACAGCGTGACCCCTGAGGACACCGCCGT
GTACTACTGCGCCAGAGAAGTGACCGGCGACCTGGAAGATGCCTTCGACA
TCTGGGGCCAGGGCACCATGGTCACCGTGTCTAGCGGAGGCGGCGGAAGC
GGTGGAGGCGGTAGCGGCGGTGGCGGTTCCGACATCCAGATGATCCAGAG
CCCTAGCTCCCTGAGCGCCAGCGTGGGCGACAGAGTGACCATCACCTGTC
GGGCCAGCCAGACCATCTGGTCCTACCTGAATTGGTATCGGCAGCGGCCA
GGCGAGGCCCCTAACCTGCTGATCTATGCCGCCAGCAGCCTGCAGAGCGG
CGTGCCAAGCAGATTCTCTGGCAGAGGCTCCGGCACCGACTTCACCCTGA
CAATCAGTTCCCTGCAGGCCGAGGACTTCGCCACCTACTACTGCCAGCAG
TCCTACAGCATCCCTCAGACCTTCGGCCAGGGGACCAAGCTGGAAATCAA
GTCTAGAATCGAAGTGATGTACCCTCCTCCTTACCTGGACAACGAGAAGT
CCAACGGCACCATCATCCACGTGAAGGGCAAGCACCTGTGTCCTTCTCCA
CTGTTCCCCGGACCTAGCAAGCCTTTCTGGGTGCTCGTTGTTGTTGGCGG
CGTGCTGGCCTGTTACAGCCTGCTGGTTACCGTGGCCTTCATCATCTTTT
GGGTCAAGAACCGGAAGGCCAAGGCCAAGCCIGTGACAAGAGGTGCTGGT
GCTGGCGGCAGACAGAGAGGCCAGAACAAAGAAAGACCTCCTCCTGTGCC
TAATCCTGACTACGAGCCCATCCGGAAGGGCCAGAGAGATCTGTATTCTG
GCCTGTGA

In some embodiments, the anti-CD22 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO 97.

An exemplary anti-CD22 CAR “CAR2”, “CAR2-3” or “CD22 CAR” polynucleotide sequence is shown below.

(SEQ ID NO: 104)
ATGGCTCTGCCTGTGACAGCTCTGCTGCTGCCTCTGGCCCTGCTGCTCCA
TGCTGCTAGACCTGACTACAAAGACGATGACGACAAGCAGGTGCAGCTCC
AGCAGTCTGGCCCAGGAATGGTCAAGCCTAGCCAGACCCTGAGCCTGACC
TGCGCCATCAGCGGCGACAGCGTGTCCTCTAACAGCGTCGCCTGGAACTG
GATCAGACAGAGCCCCAGCAGAGGCCTGGAATGGCTGGGCCGGACCTACT
ACCGGTCCACGTGGTACAACGACTACGCCGTGTCCATGAAGTCCCGGATC
ACCATCAACCCCGACACCAACAAGAACCAGTTCTCCCTGCAGCTGAACAG
CGTGACCCCTGAGGACACCGCCGTGTACTACTGCGCCAGAGAAGTGACCG
GCGACCTGGAAGATGCCTTCGACATCTGGGGCCAGGGCACCATGGTCACC
GTGTCTAGCGGAGGCGGCGGAAGCGGTGGAGGCGGTAGCGGCGGTGGCGG
TTCCGACATCCAGATGATCCAGAGCCCTAGCTCCCTGAGCGCCAGCGTGG
GCGACAGAGTGACCATCACCTGTCGGGCCAGCCAGACCATCTGGTCCTAC
CTGAATTGGTATCGGCAGCGGCCAGGCGAGGCCCCTAACCTGCTGATCTA
TGCCGCCAGCAGCCTGCAGAGCGGCGTGCCAAGCAGATTCTCTGGCAGAG
GCTCCGGCACCGACTTCACCCTGACAATCAGTTCCCTGCAGGCCGAGGAC
TTCGCCACCTACTACTGCCAGCAGTCCTACAGCATCCCTCAGACCTTCGG
CCAGGGGACCAAGCTGGAAATCAAGTCTAGAATCGAAGTGATGTACCCTC
CTCCTTACCTGGACAACGAGAAGTCCAACGGCACCATCATCCACGTGAAG
GGCAAGCACCTGTGTCCTTCTCCACTGTTCCCCGGACCTAGCAAGCCTTT
CTGGGTGCTCGTTGTTGTTGGCGGCGTGCTGGCCTGTTACAGCCTGCTGG
TTACCGTGGCCTTCATCATCTTTTGGGTCAAGAACCGGAAGGCCAAGGCC
AAGCCTGTGACAAGAGGTGCTGGTGCTGGCGGCAGACAGAGAGGCCAGAA
CAAAGAAAGACCTCCTCCTGTGCCTAATCCTGACTACGAGCCCATCCGGA
AGGGCCAGAGAGATCTGTATTCTGGCCTGTGA

In some embodiments, the anti-CD22 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 104.

### Exemplary Anti-CD22-CD3e CARs (Second CARs)

An exemplary anti-CD22 CAR “CAR2”, “CAR2-2” or “CD22 CAR” amino acid sequence is shown below. (CD8α signal peptide, high affinity CD22 scFv (m971), CD28 hinge, CD28 transmembrane domain. CD3e signaling domain)

(SEQ ID NO: 90)
MALPVTALLIPLALLLHAARPQVQLQQSGPGMVKPSQTLSLTCAISGDSV
SSNSVAWNWIRQSPSRGLEWLGRTYYRSTWYNDYAVSMKSRITINPDTNK
NQFSLQLNSVTPEDTAVYYCAREVTGDLEDAFDIWGQGTMVTVSSGGGGG
GGGSGGGGSDIQMIQSPSSLSASVGDRVTITCRASQTIWSYLNWYRQRPG
EAPNLLIYAASSLQSGVPSRFSGRGSGTDFTLTISSLQAEDFATYYCQQS
YSIPQTFGQGTKLEIKSRIEVMYPPPYLDNEKSNGTITHVKGKHLCPSPL
FPGPSKPFWVLVVVGGVLACYSLLVTVAFIIFWVKNRKAKAKPVTRGAGA
GGRQRGQNKERPPPVPNPDYEPIRKGQRDLYSGL

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO. 90.

An exemplary anti-CD22 CAR “CAR2”, “CAR2-3” or “CD22 CAR” amino acid sequence is shown below. (CD8α signal Peptide, FLAG-tag, high affinity CD22 scFv (m971), CD28 hinge, CD28 transmembrane domain, CD3e signaling domain)

(SEQ ID NO: 103)
MALPVTALLEPLALLLHAARPDYKDDDDKQVQLQQSGPGMVKPSQTLSLT
CAISGDSVSSNSVAWNWIRQSPSRGLEWLGRTYYRSTWYNDYAVSMKSRI
TINPDTNKNQFSLQLNSVTPEDTAVYYCAREVTGDLEDAFDIWGQGTMVT
VSSGGGGGGGGGGGGGSDIQMIQSPSSLSASVGDRVTITCRASQTIWSYL
NWYRQRPGEAPNLLIYAASSLQSGVPSRFSGRGSGTDFTLTISSLQAEDF
ATYYCQQSYSIPQTFGQGTKLEIKSRIEVMYPPPYLDNEKSNGTIIHVKG
KHLCPSPLFPGPSKPFWVLVVVGGVLACYSLLVTVAFIIFWVKNRKAKAK
PVIRGAGAGGRQRGQNKERPPPVPNPDYEPIRKGQRDLYSGE

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO 103.

### ii) Exemplary Anti-CD19 CAR Constructs (Second CARs)

Disclosed herein are CARs that specifically bind to CD19. In some embodiments, the CAR comprises an antigen recognition domain that specifically binds human CD19, a hinge domain comprising or consisting of a CD28 hinge domain, a transmembrane domain comprising or consisting of a CD3ε transmembrane domain; and an intracellular signaling domain comprising or consisting of a CD3ε intracellular signaling domain. Also disclosed herein are nucleic acid sequences encoding said CARs. In some embodiments, a T cell or population of T cells described herein is genetically modified to express at least one of the exemplary anti-CD19 CAR constructs described herein.

An exemplary anti-CD19 CAR “CAR2” or “CD19 CAR” amino acid sequence is shown below. (IgG signal peptide. CD19 scFv (FMC63), CD28 hinge, CD3ε transmembrane domain. CD3ε signaling domain)

(SEQ ID NO: 59)
MEFGLSWLFLVAILKGVQCSRDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGSTSGSGKPGSGEGSTKGEVKL
QESGPGLVAPSQSLSVTCTVSGVSLPDYGVSWIRQPPRKGLEWLGVIWGS
ETTYYNSALKSRLTIIKDNSKSQVFLKMNSLQTDDTAIYYCAKHYYYGGS
YAMDYWGQGTSVTVSRIEVMYPPPYLDNEKSNGTIIHVKGKHLCPSPLFP
GPSKPVMSVATIVIVDICITGGLLLLVYYWSKNRKAKAKPVTRGAGAGGR
QRGQNKERPPPVPNPDYEPIRKGQRDLYSGL

In some embodiments, the anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO. 59.

An exemplary anti-CD19 CAR “CAR2” or “CD19 CAR” polynucleotide sequence is shown below. (IgG signal peptide. CD19 scFv (FMC63), CD28 hinge, CD3ε transmembrane domain, CD3ε signaling domain)

(SEQ ID NO: 60)
ATGGAGTTTGGCCTGAGCTGGCTGTTCCTGGTGGCCATCCTCAAGGGCGT
GCAGTGCTCCAGGGACATCCAGATGACCCAGACCACAAGCAGCCTGAGCG
CTTCCCTCGGCGACAGGGTGACCATCTCCTGTAGAGCCTCCCAAGACATC
TCCAAGTACCTGAACTGGTACCAGCAGAAACCCGACGGCACCGTGAAGCT
GCTGATCTACCACACCAGCAGGCTGCATTCCGGCGTGCCCTCCAGATTTT
CCGGCAGCGGCTCTGGTACCGACTACAGCCTCACCATCAGCAACTTAGAA
CAGGAGGACATCGCCACATATTTCTGCCAACAGGGAAACACACTCCCCTA
TACCTTCGGCGGCGGCACAAAGTTAGAAATCACCGGCTCCACATCCGGCA
GCGGAAAACCTGGTTCTGGCGAGGGCAGCACCAAGGGCGAAGTGAAGCTG
CAGGAAAGCGGACCTGGACTGGTCGCTCCCAGCCAGAGCCTCAGCGTGAC
CTGTACAGTGAGCGGCGTGAGCCTGCCTGATTACGGCGTGAGCTGGATTA
GACAGCCTCCCAGGAAGGGCTTAGAATGGCTCGGCGTGATTTGGGGCAGC
GAGACAACCTACTATAACAGCGCCCTGAAGAGCAGGCTCACCATTATCAA
GGACAACAGCAAATCCCAGGTCTTCCTGAAGATGAACAGCCTCCAGACCG
ACGACACCGCCATCTACTACTGCGCCAAGCACTACTATTATGGCGGCTCC
TACGCCATGGACTACTGGGGCCAGGGCACCAGCGTGACAGTGTCTAGAAT
CGAAGTGATGTACCCTCCACCTTACCTGGACAACGAGAAGTCCAACGGCA
CCATCATCCACGTGAAGGGCAAGCACCTGTGTCCTTCTCCACTGTTCCCC
GGACCTAGCAAGCCTGTGATGAGCGTGGCCACCATCGTGATCGTGGACAT
CTGTATCACCGGCGGACTGCTGCTGCTGGTGTACTACTGGTCCAAGAACC
GGAAGGCCAAGGCCAAGCCAGTGACAAGAGGTGCTGGTGCTGGCGGCAGA
CAGAGAGGCCAGAACAAAGAAAGACCTCCTCCTGTGCCTAATCCTGACTA
CGAGCCCATCCGGAAGGGCCAGAGAGATCTGTATAGCGGCCTCTGA

In some embodiments, the anti-CD19 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 60.

Cleavage sequences can be used to create linked- or co-expression of genes in the constructs provided in the present disclosure. For example, cleavage sequences could be used to co-express genes (e.g. CAR1 and CAR2) by linking open reading frames to form a single cistron (e.g bicistronic CAR). In some aspects, cleavage sequences can comprise 2A self-cleaving peptide sequence elements. Exemplary 2A self-cleaving peptide sequence elements include but are not limited to T2A, P2A, E2A and F2A. In some embodiments, the cleavage sequence comprises a P2A sequence. In some embodiments, a cleavage sequence can comprise a furin cleavage peptide. In some embodiments, a cleavage sequence can comprise a furin cleavage peptide and a P2A sequence.

In some embodiments, P2A comprises or consists of an amino acid sequence having at least 90%, 91%, 92%, 93%, 94%, 95%, 96%, 97%, 98%, 99% or 100% identity with the amino acid sequence of GSGATNFSLLKQAGDVEENPGP (SEQ ID NO: 61).

In some embodiments, P2A comprises or consists of an amino acid sequence having at least 90%, 91%, 92%, 93%, 94%, 95%, 96%, 97%, 98%, 99% or 100% identity with the amino acid sequence of GSGATNFSLLKQAGDVEENPGP (SEQ ID NO: 62).

In some embodiments, T2A comprises or consists of an amino acid sequence having at least 90%, 91%, 92%, 93%, 94%, 95%, 96%, 97%, 98%, 99% or 100% identity with the amino acid sequence of GSGEGRGSLLTCGDVEENPGP (SEQ ID NO. 63).

In some embodiments, E2A comprises or consists of an amino acid sequence having at least 90%, 91%, 92%, 93%, 94%, 95%, 96%, 97%, 98%, 99% or 100% identity with the amino acid sequence of GSGQCTNYALLKLAGDVESNPGP (SEQ ID NO: 64).

In some embodiments, F2A comprises or consists of an amino acid sequence having at least 90%, 91%, 92%, 93%, 94%, 95%, 96%, 97%, 98%, 99% or 100% identity with the amino acid sequence of GSGVKQTLNFDLLKLAGDVESNPGP (SEQ ID NO: 65).

In some embodiments, a furin cleavage peptide comprises or consists of an amino acid sequence having at least 90%, 91%, 92%, 93%, 94%, 95%, 96%, 97%, 98%, 99% or 100% identity with the amino acid sequence of RKRRGSGTPDPW (SEQ ID NO. 66).

In some embodiments, a cleavage sequence comprises or consists of an amino acid sequence having at least 90%, 91%, 92%, 93%, 94%, 95%, 96%, 97%, 98%, 99% or 100% identity with the amino acid sequence of RKRRGSGTPDPWGSGATNFSLLKQAGDVEENPGP (SEQ ID NO. 67).

In some embodiments, the CARS described herein can be under the control of an inducible promoter for gene transcription. In some embodiments, the inducible promoter is an EF1a promoter. In some embodiments, the inducible promoter is a PGK promoter.

Exemplary sequences of constructs disclosed herein comprising an anti-CD22 CAR and an anti-CD19 CAR are shown below.

iii) Exemplary Bicistronic CAR Constructs

An exemplary bicistronic anti-CD22 CAR and anti-CD19 CAR “CAR1-linker-CAR2”, “CD3ε-CAR”, “CD3e CAR” or “22×19CD3e CAR” amino acid sequence is shown below (CAR1; Furin/P2A linker; CAR2).

(SEQ ID NO: 68)
ASATMALPVTALLLPLALLLHAARPQVQLQQSGPGLVKPSQTLSLTCAIS
GDSVSSNSAAWNWIRQSPSRGLEWLGRTYYRSKWYNDYAVSVKSRITINP
DTSKNQFSLQLNSVTPEDTAVYYCAREVTGDLEDAFDIWGQGTMVTVSSG
GGGSDIQMTQSPSSLSASVGDRVTITCRASQTIWSYLNWYQQRPGKAPNL
LIYAASSLQSGVPSRESGRGSGTDFTLTISSLQAEDFATYYCQQSYSIPQ
TFGQGTKLEIKLETTTPAPRPPTPAPTIASQPLSLRPEACRPAAGGAVHT
RGLDFACDIYIWAPLAGTCGVILLSLVITLYCKRGRKKLLYIFKQPFMRP
VQTTQEEDGCSCRFPEEEEGGCELDIRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPRRKRRGSGTPDPW
GSGATNFSLLKQAGDVEENPGPGSMEFGLSWLFLVAILKGVQCSRDIQMT
QTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLE
ITGSTSGSGKPGSGEGSTKGEVKLQESGPGLVAPSQSLSVTCTVSGVSLP
DYGVSWIRQPPRKGLEWLGVIWGSETTYYNSALKSRLTIIKDNSKSQVFL
KMNSLQTDDTAIYYCAKHYYYGGSYAMDYWGQGTSVTVSRIEVMYPPPYL
DNEKSNGTIIHVKGKHLCPSPLFPGPSKPVMSVATIVIVDICITGGLLLL
VYYWSKNRKAKAKPVTRGAGAGGRQRGQNKERPPPVPNPDYEPIRKGQRD
LYSGL

In some embodiments, the bicistronic anti-CD22 CAR and anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 68.

An exemplary bicistronic anti-CD22 CAR and anti-CD19 CAR “CAR1-linker-CAR2”, “CD3ε-CAR”, “CD3e CAR”, or “22×19CD3e CAR” amino acid sequence is shown below (CAR1; Furin/P2A linker; CAR2).

(SEQ ID NO: 88)
MALPVTALLLPLALLLHAARPQVQLQQSGPGLVKPSQTLSLTCAISGDSV
SSNSAAWNWIRQSPSRGLEWLGRTYYRSKWYNDYAVSVKSRITINPDTSK
NQFSLQLNSVTPEDTAVYYCAREVTGDLEDAFDIWGQGTMVTVSSGGGGS
DIQMTQSPSSISASVGDRVTITCRASQTIWSYLNWYQQRPGKAPNLLIYA
ASSLQSGVPSRESGRGSGTDFTLTISSLQAEDFATYYCQQSYSIPQTFGQ
GTKLEIKLETTTPAPRPPTPAPTIASQPLSLRPEACRPAAGGAVHTRGLD
FACDIYIWAPLAGTCGVLLLSLVITLYCKRGRKKLLYIFKQPFMRPVQTT
QEEDGCSCRFPEEEEGGCELDIRVKFSRSADAPAYQQGQNQLYNELNLGR
REEYDVLDKRRGRDPEMGGKPRRKNPQEGLYNELQKDKMAEAYSEIGMKG
ERRRGKGHDGLYQGLSTATKDTYDALHMQALPPRRKRRGSGTPDPWGSGA
TNFSLLKQAGDVEENPGPGSMEFGLSWLFLVAILKGVQCSRDIQMTQTTS
SLSASLGDRVTISCRASQDISKYLNWYQQKPDGTVKLLIYHTSRLHSGVP
SRFSGSGSGTDYSLTISNLEQEDIATYFCQQGNTLPYTFGGGTKLEITGS
TSGSGKPGSGEGSTKGEVKLQESGPGLVAPSQSLSVTCTVSGVSLPDYGV
SWIRQPPRKGLEWLGVIWGSETTYYNSALKSRLTIIKDNSKSQVELKMNS
LQTDDTAIYYCAKHYYYGGSYAMDYWGQGTSVTVSRIEVMYPPPYLDNEK
SNGTIIHVKGKHLCPSPLFPGPSKPVMSVATIVIVDICITGGLLLLVYYW
SKNRKAKAKPVTRGAGAGGRQRGQNKERPPPVPNPDYEPIRKGQRDLYSG
L

In some embodiments, the bicistronic anti-CD22 CAR and anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 88.

An exemplary bicistronic anti-CD22 CAR and anti-CD19 CAR “CAR1-linker-CAR2”, “CD3ε-CAR”, “CD3e CAR”, or “22×19CD3e CAR” polynucleotide sequence is shown below. (CAR1; furin/P2A linker; CAR2).

(SEQ ID NO: 69)
GCTAGCGCCACCATGGCTCTGCCTGTGACAGCTCTGCTGCTGCCTCTGGC
CCTGCTGCTCCATGCTGCTAGACCTCAGGTGCAGCTCCAGCAGTCTGGCC
CAGGACTGGTCAAGCCTAGCCAGACCCTGAGCCTGACCTGCGCCATCAGC
GGCGACAGCGTGTCCTCTAACAGCGCCGCCTGGAACTGGATCAGACAGAG
CCCCAGCAGAGGCCTGGAATGGCTGGGCCGGACCTACTACCGGTCCAAGT
GGTACAACGACTACGCCGTGTCCGTGAAGTCCCGGATCACCATCAACCCC
GACACCAGCAAGAACCAGTTCTCCCTGCAGCTGAACAGCGTGACCCCTGA
GGACACCGCCGTGTACTACTGCGCCAGAGAAGTGACCGGCGACCTGGAAG
ATGCCTTCGACATCTGGGGCCAGGGCACCATGGTCACCGTGTCTAGCGGA
GGCGGCGGAAGCGACATCCAGATGACCCAGAGCCCTAGCTCCCTGAGCGC
CAGCGTGGGCGACAGAGTGACCATCACCTGTCGGGCCAGCCAGACCATCT
GGTCCTACCTGAATTGGTATCAGCAGCGGCCAGGCAAGGCCCCTAACCTG
CTGATCTATGCCGCCAGCAGCCTGCAGAGCGGCGTGCCAAGCAGATTCTC
TGGCAGAGGCTCCGGCACCGACTTCACCCTGACAATCAGTTCCCTGCAGG
CCGAGGACTTCGCCACCTACTACTGCCAGCAGTCCTACAGCATCCCTCAG
ACCTTCGGCCAGGGGACCAAGCTGGAAATCAAGCTCGAGACCACCACCCC
CGCCCCTAGGCCTCCCACACCTGCCCCCACAATCGCCTCCCAGCCTCTCA
GCCTGAGGCCTGAAGCTTGCAGGCCCGCTGCCGGAGGAGCTGTCCATACC
AGGGGACTCGACTTCGCCTGCGACATTTACATTTGGGCCCCTCTGGCTGG
AACCTGCGGAGTCCTGCTGCTGTCCCTGGTGATCACACTGTACTGTAAGA
GGGGCAGAAAGAAGCTGCTCTACATCTTCAAGCAGCCCTTTATGAGACCC
GTGCAGACAACCCAGGAGGAAGACGGATGCAGCTGCAGGTTCCCTGAGGA
GGAGGAGGGCGGCTGCGAACTGGATATCAGGGTGAAGTTCAGCAGGAGCG
CCGACGCCCCCGCTTATCAACAGGGCCAGAACCAGCTGTACAACGAGCTG
AACCTCGGCAGAAGAGAGGAGTATGACGTGCTGGACAAGAGGAGGGGCAG
GGACCCTGAGATGGGCGGCAAGCCTAGAAGAAAGAACCCCCAGGAAGGCC
TCTACAACGAACTGCAGAAGGACAAGATGGCCGAGGCCTACAGCGAGATC
GGCATGAAAGGCGAGAGAAGGAGGGGAAAGGGACATGACGGCCTGTACCA
GGGACTCTCCACAGCCACCAAGGACACCTACGATGCCCTGCACATGCAGG
CTCTGCCCCCTAGAAGGAAGAGAAGAGGCTCTGGTACCCCCGATCCTTGG
GGAAGCGGCGCTACCAACTTCTCCCTGCTCAAGCAGGCTGGCGATGTGGA
GGAGAACCCCGGCCCCGGATCCATGGAGTTTGGCCTGAGCTGGCTGTTCC
TGGTGGCCATCCTCAAGGGCGTGCAGTGCTCCAGGGACATCCAGATGACC
CAGACCACAAGCAGCCTGAGCGCTTCCCTCGGCGACAGGGTGACCATCTC
CTGTAGAGCCTCCCAAGACATCTCCAAGTACCTGAACTGGTACCAGCAGA
AACCCGACGGCACCGTGAAGCTGCTGATCTACCACACCAGCAGGCTGCAT
TCCGGCGTGCCCTCCAGATTTTCCGGCAGCGGCTCTGGTACCGACTACAG
CCTCACCATCAGCAACTTAGAACAGGAGGACATCGCCACATATTTCTGCC
AACAGGGAAACACACTCCCCTATACCTTCGGCGGCGGCACAAAGTTAGAA
ATCACCGGCTCCACATCCGGCAGCGGAAAACCTGGTTCTGGCGAGGGCAG
CACCAAGGGCGAAGTGAAGCTGCAGGAAAGCGGACCTGGACTGGTCGCTC
CCAGCCAGAGCCTCAGCGTGACCTGTACAGTGAGCGGCGTGAGCCTGCCT
GATTACGGCGTGAGCTGGATTAGACAGCCTCCCAGGAAGGGCTTAGAATG
GCTCGGCGTGATTTGGGGCAGCGAGACAACCTACTATAACAGCGCCCTGA
AGAGCAGGCTCACCATTATCAAGGACAACAGCAAATCCCAGGTCTTCCTG
AAGATGAACAGCCTCCAGACCGACGACACCGCCATCTACTACTGCGCCAA
GCACTACTATTATGGCGGCTCCTACGCCATGGACTACTGGGGCCAGGGCA
CCAGCGTGACAGTGTCTAGAATCGAAGTGATGTACCCTCCACCTTACCTG
GACAACGAGAAGTCCAACGGCACCATCATCCACGTGAAGGGCAAGCACCT
GTGTCCTTCTCCACTGTTCCCCGGACCTAGCAAGCCTGTGATGAGCGTGG
CCACCATCGTGATCGTGGACATCTGTATCACCGGCGGACTGCTGCTGCTG
GTGTACTACTGGTCCAAGAACCGGAAGGCCAAGGCCAAGCCAGTGACAAG
AGGTGCTGGTGCTGGCGGCAGACAGAGAGGCCAGAACAAAGAAAGACCTC
CTCCTGTGCCTAATCCTGACTACGAGCCCATCCGGAAGGGCCAGAGAGAT
CTGTATAGCGGCCTC

In some embodiments, the bicistronic anti-CD22 CAR and anti-CD19 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 69.

An exemplary bicistronic anti-CD22 CAR and anti-CD19 CAR “CAR1-linker-CAR2”, “CD3ε-CAR”, “CD3e CAR”, or “22×19CD3e CAR” polynucleotide sequence is shown below. (CAR1; furin/P2A linker; CAR2).

(SEQ ID NO: 89)
ATGGCTCTGCCTGTGACAGCTCTGCTGCTGCCTCTGGCCCTGCTGCTCCA
TGCTGCTAGACCTCAGGTGCAGCTCCAGCAGTCTGGCCCAGGACTGGTCA
AGCCTAGCCAGACCCTGAGCCTGACCTGCGCCATCAGCGGCGACAGCGTG
TCCTCTAACAGCGCCGCCTGGAACTGGATCAGACAGAGCCCCAGCAGAGG
CCTGGAATGGCTGGGCCGGACCTACTACCGGTCCAAGTGGTACAACGACT
ACGCCGTGTCCGTGAAGTCCCGGATCACCATCAACCCCGACACCAGCAAG
AACCAGTTCTCCCTGCAGCTGAACAGCGTGACCCCTGAGGACACCGCCGT
GTACTACTGCGCCAGAGAAGTGACCGGCGACCTGGAAGATGCCTTCGACA
TCTGGGGCCAGGGCACCATGGTCACCGTGTCTAGCGGAGGCGGCGGAAGC
GACATCCAGATGACCCAGAGCCCTAGCTCCCTGAGCGCCAGCGTGGGCGA
CAGAGTGACCATCACCTGTCGGGCCAGCCAGACCATCTGGTCCTACCTGA
ATTGGTATCAGCAGCGGCCAGGCAAGGCCCCTAACCTGCTGATCTATGCC
GCCAGCAGCCTGCAGAGCGGCGTGCCAAGCAGATTCTCTGGCAGAGGCTC
CGGCACCGACTTCACCCTGACAATCAGTTCCCTGCAGGCCGAGGACTTCG
CCACCTACTACTGCCAGCAGTCCTACAGCATCCCTCAGACCTTCGGCCAG
GGGACCAAGCTGGAAATCAAGCTCGAGACCACCACCCCCGCCCCTAGGCC
TCCCACACCTGCCCCCACAATCGCCTCCCAGCCTCTCAGCCTGAGGCCTG
AAGCTTGCAGGCCCGCTGCCGGAGGAGCTGTCCATACCAGGGGACTCGAC
TTCGCCTGCGACATTTACATTTGGGCCCCTCTGGCTGGAACCTGCGGAGT
CCTGCTGCTGTCCCTGGTGATCACACTGTACTGTAAGAGGGGCAGAAAGA
AGCTGCTCTACATCTTCAAGCAGCCCTTTATGAGACCCGTGCAGACAACC
CAGGAGGAAGACGGATGCAGCTGCAGGTTCCCTGAGGAGGAGGAGGGCGG
CTGCGAACTGGATATCAGGGTGAAGTTCAGCAGGAGCGCCGACGCCCCCG
CTTATCAACAGGGCCAGAACCAGCTGTACAACGAGCTGAACCTCGGCAGA
AGAGAGGAGTATGACGTGCTGGACAAGAGGAGGGGCAGGGACCCTGAGAT
GGGCGGCAAGCCTAGAAGAAAGAACCCCCAGGAAGGCCTCTACAACGAAC
TGCAGAAGGACAAGATGGCCGAGGCCTACAGCGAGATCGGCATGAAAGGC
GAGAGAAGGAGGGGAAAGGGACATGACGGCCTGTACCAGGGACTCTCCAC
AGCCACCAAGGACACCTACGATGCCCTGCACATGCAGGCTCTGCCCCCTA
GAaggaagagaagaggctctggtacccccgatccttggggaagcggcgct
accaacttctccctgctcaagcaggctggcgatgtggaggagaaccccgg
ccccGGATCCATGGAGTTTGGCCTGAGCTGGCTGTTCCTGGTGGCCATCC
TCAAGGGCGTGCAGTGCTCCAGGGACATCCAGATGACCCAGACCACAAGC
AGCCTGAGCGCTTCCCTCGGCGACAGGGTGACCATCTCCTGTAGAGCCTC
CCAAGACATCTCCAAGTACCTGAACTGGTACCAGCAGAAACCCGACGGCA
CCGTGAAGCTGCTGATCTACCACACCAGCAGGCTGCATTCCGGCGTGCCC
TCCAGATTTTCCGGCAGCGGCTCTGGTACCGACTACAGCCTCACCATCAG
CAACTTAGAACAGGAGGACATCGCCACATATTTCTGCCAACAGGGAAACA
CACTCCCCTATACCTTCGGCGGCGGCACAAAGTTAGAAATCACCGGCTCC
ACATCCGGCAGCGGAAAACCTGGTTCTGGCGAGGGCAGCACCAAGGGCGA
AGTGAAGCTGCAGGAAAGCGGACCTGGACTGGTCGCTCCCAGCCAGAGCC
TCAGCGTGACCTGTACAGTGAGCGGCGTGAGCCTGCCTGATTACGGCGTG
AGCTGGATTAGACAGCCTCCCAGGAAGGGCTTAGAATGGCTCGGCGTGAT
TTGGGGCAGCGAGACAACCTACTATAACAGCGCCCTGAAGAGCAGGCTCA
CCATTATCAAGGACAACAGCAAATCCCAGGTCTTCCTGAAGATGAACAGC
CTCCAGACCGACGACACCGCCATCTACTACTGCGCCAAGCACTACTATTA
TGGCGGCTCCTACGCCATGGACTACTGGGGCCAGGGCACCAGCGTGACAG
TGTCTAGAATCGAAGTGATGTACCCTCCACCTTACCTGGACAACGAGAAG
TCCAACGGCACCATCATCCACGTGAAGGGCAAGCACCTGTGTCCTTCTCC
ACTGTTCCCCGGACCTAGCAAGCCTGTGATGAGCGTGGCCACCATCGTGA
TCGTGGACATCTGTATCACCGGCGGACTGCTGCTGCTGGTGTACTACTGG
TCCAAGAACCGGAAGGCCAAGGCCAAGCCAGTGACAAGAGGTGCTGGTGC
TGGCGGCAGACAGAGAGGCCAGAACAAAGAAAGACCTCCTCCTGTGCCTA
ATCCTGACTACGAGCCCATCCGGAAGGGCCAGAGAGATCTGTATAGCGGC
CTC

In some embodiments, the bicistronic anti-CD22 CAR and anti-CD19 CAR provided herein is encoded by a polynucleotide sequence comprising or consisting of an nucleic acid sequence having at least 90, 91%, 92%, 93%, 94%, 95%, 96%, 97%, 98%, 99% or 100% identity with the nucleic acid sequence of SEQ ID NO: 89.

An exemplary bicistronic anti-CD22 CAR and anti-CD22 CAR with 6-His and FLAG tags “CAR1-linker-CAR2”, “CD3ε-CAR”, “CD3e CAR”, or “22×22CD3e CAR” amino acid sequence is shown below. (CAR1; furin/P2A linker; CAR2).

(SEQ ID NO: 93)
MALPVTALLLPLALLLHAARPHHHHHHQVQLQQSGPGMVKPSQTLSLTCA
ISGDSVSSNSVAWNWIRQSPSRGLEWLGRTYYRSTWYNDYAVSMKSRITI
NPDINKNQFSLQINSVTPEDTAVYYCAREVIGDLEDAFDIWGQGTMVTVS
SGGGGGGGGSGGGGSDIQMIQSPSSLSASVGDRVTITCRASQTIWSYLNW
YRQRPGEAPNLLIYAASSLQSGVPSRESGRGSGTDFTLTISSLQAEDFAT
YYCQQSYSIPQTFGQGTKLEIKLETTTPAPRPPTPAPTIASQPLSIRPEA
CRPAAGGAVHTRGLDFACDIYIWAPLAGTCGVLLLSLVITLYCKRGRKKL
LYIFKQPFMRPVQTTQEEDGCSCREPEEEEGGCELDIRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPRR
KRRGSGTPDPWGSGATNFSLLKQAGDVEENPGPGSMALPVTALLLPLALL
LHAARPDYKDDDDKQVQLQQSGPGMVKPSQTLSLTCAISGDSVSSNSVAW
NWIRQSPSRGLEWIGRTYYRSTWYNDYAVSMKSRITINPDTNKNQFSLQL
NSVTPEDTAVYYCAREVTGDLEDAFDIWGQGTMVTVSSGGGGSGGGGSGG
GGSDIQMIQSPSSLSASVGDRVTITCRASQTIWSYLNWYRQRPGEAPNLL
IYAASSLQSGVPSRFSGRGSGTDFTLTISSLQAEDFATYYCQQSYSIPQT
FGQGTKLEIKSRIEVMYPPPYLDNEKSNGTIIHVKGKHICPSPLFPGPSK
PFWVLVVVGGVLACYSLLVTVAFIIFWVKNRKAKAKPVTRGAGAGGRQRG
QNKERPPPVPNPDYEPIRKGQRDLYSGL

In some embodiments, the bicistronic anti-CD22 CAR and anti-CD22 CAR with 6-His and FLAG tags provided herein may comprise or consist of an amino acid sequence having at least 90%, 91%, 92%, 93%, 94%, 95%, 96%, 97%, 98%, 99% or 100% identity with the amino acid sequence of SEQ ID NO: 93.

An exemplary bicistronic anti-CD22 CAR and anti-CD22 CAR with 6-His and FLAG tags “CAR1-linker-CAR2”, “CD3ε-CAR”, “CD3e CAR”, or “22×22CD3e CAR” polynucleotide sequence is shown below.

(SEQ ID NO: 94)
ATGGCACTGCCAGTGACTGCATTACTCTTGCCACTCGCGCTACTGTTACA
CGCAGCACGTCCACATCACCATCACCATCACCAAGTCCAATTGCAACAAA
GCGGGCCGGGCATGGTGAAACCGAGTCAAACGTTATCTCTTACGTGTGCG
ATTTCGGGGGATAGTGTCAGCAGCAATTCAGTGGCGTGGAATTGGATTCG
CCAATCGCCGAGTCGCGGGTTGGAGTGGCTCGGGCGCACGTATTATCGCA
GCACATGGTATAATGATTATGCGGTCAGCATGAAAAGCCGCATTACGATT
AATCCGGATACGAACAAAAATCAATTTAGCTTACAATTAAATTCCGTCAC
GCCGGAAGATACAGCGGTCTATTATTGTGCGCGCGAGGTCACGGGGGATC
TCGAAGACGCGTTTGATATTTGGGGGCAAGGGACCATGGTGACTGTCAGC
TCTGGTGGAGGGGGCAGTGGAGGTGGGGGATCGGGAGGTGGTGGCAGTGA
TATTCAAATGATCCAAAGTCCATCCAGCCTATCCGCATCTGTCGGAGATC
GCGTAACGATTACGTGCCGCGCGAGTCAAACGATTTGGAGCTATCTGAAC
TGGTACCGGCAACGCCCGGGCGAAGCGCCGAATCTCTTGATTTACGCGGC
GTCCTCATTACAGTCGGGTGTCCCGAGCCGCTTTAGCGGCCGCGGAAGCG
GTACGGATTTTACGTTAACCATTAGCAGCCTCCAGGCGGAAGATTTTGCG
ACGTATTACTGTCAACAGAGCTATAGCATTCCGCAGACGTTTGGTCAGGG
CACGAAATTGGAGATTAAACTCGAGACCACCACCCCCGCCCCTAGGCCTC
CCACACCTGCCCCCACAATCGCCTCCCAGCCTCTCAGCCTGAGGCCTGAA
GCTTGCAGGCCCGCTGCCGGAGGAGCTGTCCATACCAGGGGACTCGACTT
CGCCTGCGACATTTACATTTGGGCCCCTCTGGCTGGAACCTGCGGAGTCC
TGCTGCTGTCCCTGGTGATCACACTGTACTGTAAGAGGGGCAGAAAGAAG
CTGCTCTACATCTTCAAGCAGCCCTTTATGAGACCCGTGCAGACAACCCA
GGAGGAAGACGGATGCAGCTGCAGGTTCCCTGAGGAGGAGGAGGGGGGCT
GCGAACTGGATATCAGGGTGAAGTTCAGCAGGAGCGCCGACGCCCCCGCT
TATCAACAGGGCCAGAACCAGCTGTACAACGAGCTGAACCTCGGCAGAAG
AGAGGAGTATGACGTGCTGGACAAGAGGAGGGGCAGGGACCCTGAGATGG
GCGGCAAGCCTAGAAGAAAGAACCCCCAGGAAGGCCTCTACAACGAACTG
CAGAAGGACAAGATGGCCGAGGCCTACAGCGAGATCGGCATGAAAGGCGA
GAGAAGGAGGGGAAAGGGACATGACGGCCTGTACCAGGGACTCTCCACAG
CCACCAAGGACACCTACGATGCCCTGCACATGCAGGCTCTGCCCCCTAGA
AGGAAGAGAAGAGGCTCTGGTACCCCCGATCCTTGGGGAAGCGGCGCTAC
CAACTTCTCCCTGCTCAAGCAGGCTGGCGATGTGGAGGAGAACCCCGGCC
CCGGATCCATGGCTCTGCCTGTGACAGCTCTGCTGCTGCCTCTGGCCCTG
CTGCTCCATGCTGCTAGACCTGACTACAAAGACGATGACGACAAGCAGGT
GCAGCTCCAGCAGTCTGGCCCAGGAATGGTCAAGCCTAGCCAGACCCTGA
GCCTGACCTGCGCCATCAGCGGCGACAGCGTGTCCTCTAACAGCGTCGCC
TGGAACTGGATCAGACAGAGCCCCAGCAGAGGCCTGGAATGGCTGGGCCG
GACCTACTACCGGTCCACGTGGTACAACGACTACGCCGTGTCCATGAAGT
CCCGGATCACCATCAACCCCGACACCAACAAGAACCAGTTCTCCCTGCAG
CTGAACAGCGTGACCCCTGAGGACACCGCCGTGTACTACTGCGCCAGAGA
AGTGACCGGCGACCTGGAAGATGCCTTCGACATCTGGGGCCAGGGCACCA
TGGTCACCGTGTCTAGCGGAGGCGGCGGAAGCGGTGGAGGCGGTAGCGGC
GGTGGCGGTTCCGACATCCAGATGATCCAGAGCCCTAGCTCCCTGAGCGC
CAGCGTGGGCGACAGAGTGACCATCACCTGTCGGGCCAGCCAGACCATCT
GGTCCTACCTGAATTGGTATCGGCAGCGGCCAGGCGAGGCCCCTAACCTG
CTGATCTATGCCGCCAGCAGCCTGCAGAGCGGCGTGCCAAGCAGATTCTC
TGGCAGAGGCTCCGGCACCGACTTCACCCTGACAATCAGTTCCCTGCAGG
CCGAGGACTTCGCCACCTACTACTGCCAGCAGTCCTACAGCATCCCTCAG
ACCTTCGGCCAGGGGACCAAGCTGGAAATCAAGTCTAGAATCGAAGTGAT
GTACCCTCCTCCTTACCTGGACAACGAGAAGTCCAACGGCACCATCATCC
ACGTGAAGGGCAAGCACCTGTGTCCTTCTCCACTGTTCCCCGGACCTAGC
AAGCCTTTCTGGGTGCTCGTTGTTGTTGGCGGCGTGCTGGCCTGTTACAG
CCTGCTGGTTACCGTGGCCTTCATCATCTTTTGGGTCAAGAACCGGAAGG
CCAAGGCCAAGCCTGTGACAAGAGGTGCTGGTGCTGGCGGCAGACAGAGA
GGCCAGAACAAAGAAAGACCTCCTCCTGTGCCTAATCCTGACTACGAGCC
CATCCGGAAGGGCCAGAGAGATCTGTATTCTGGCCTGTGAG

In some embodiments, the bicistronic anti-CD22 CAR and anti-CD22 CAR with 6-His and FLAG tags provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 94.

An exemplary bicistronic anti-CD22 CAR and anti-CD22 CAR (without 6-His and FLAG tags) “CAR1-linker-CAR2”, “CD3ε-CAR”, “CD3e CAR”, or “22×22CD3e CAR” amino acid sequence is shown below. (CAR1; furin/P2A linker; CAR2).

(SEQ ID NO: 95)
MALPVTALLLPLALLLHAARPQVQLQQSGPGMVKPSQTLSLTCAISGDSV
SSNSVAWNWIRQSPSRGLEWLGRTYYRSTWYNDYAVSMKSRITINPDTNK
NQFSLQLNSVTPEDTAVYYCAREVTGDLEDAFDIWGQGIMVTVSSGGGGS
GGGGSGGGGSDIQMIQSPSSISASVGDRVTITCRASQTIWSYLNWYRQRP
GEAPNLLIYAASSLQSGVPSRESGRGSGTDFTLTISSLQAEDFATYYCQQ
SYSIPQTFGQGTKLEIKLETTTPAPRPPTPAPTIASQPLSLRPEACRPAA
GGAVHTRGLDFACDTYIWAPLAGTCGVLLLSLVITLYCKRGRKKLLYIFK
QPFMRPVQTTQEEDGCCRFPEEEEGGCELDIRVKFSRSADAPAYQQGQNQ
LYNELNLGRREEYDVLDKRRGRDPEMGGKPRRKNPQEGLYNELQKDKMAE
AYSEIGMKGERRRGKGHDGLYQGLSTATKDTYDALHMQALPPRRKRRGSG
TPDPWGSGATNFSLLKQAGDVEENPGPGSMALPVTALLLPLALLLHAARP
QVQLQQSGPGMVKPSQTLSLTCAISGDSVSSNSVAWNWIRQSPSRGLEWL
GRTYYRSTWYNDYAVSMKSRITINPDTNKNQFSLQINSVTPEDTAVYYCA
REVIGDLEDAFDIWGQGTMVTVSSGGGGSGGGGGGGGSDIQMIQSPSSIS
ASVGDRVTITCRASQTIWSYLNWYRQRPGEAPNLLIYAASSLQSGVPSRE
SGRGSGTDFTLTISSLQAEDFATYYCQQSYSIPQTFGQGTKLEIKSRIEV
MYPPPYLDNEKSNGTIIHVKGKHLCPSPLFPGPSKPFWVLVVVGGVLACY
SLLVTVAFIIFWVKNRKAKAKPVTRGAGAGGRQRGQNKERPPPVPNPDYE
PIRKGQRDLYSGL

In some embodiments, the bicistronic anti-CD22 CAR and anti-CD22 CAR (without 6-His and FLAG tags) provided herein may comprise or consist of an amino acid sequence having at least 90%, 91%, 92%, 93%, 94%, 95%, 96%, 97%, 98%, 99% or 100% identity with the amino acid sequence of SEQ ID NO: 95.

An exemplary bicistronic anti-CD22 CAR and anti-CD22 CAR (without 6-His and FLAG tags) “CAR1-linker-CAR2”, “CD3ε-CAR”, “CD3e CAR”, or “22×22CD3e CAR” polynucleotide sequence is shown below.

(SEQ ID NO: 96)
ATGGCACTGCCAGTGACTGCATTACTCTTGCCACTCGCGCTACTGTTACA
CGCAGCACGTCCACAAGTCCAATTGCAACAAAGCGGGCCGGGCATGGTGA
AACCGAGTCAAACGTTATCTCTTACGTGTGCGATTTCGGGGGATAGTGTC
AGCAGCAATTCAGTGGCGTGGAATTGGATTCGCCAATCGCCGAGTCGCGG
GTTGGAGTGGCTCGGGCGCACGTATTATCGCAGCACATGGTATAATGATT
ATGCGGTCAGCATGAAAAGCCGCATTACGATTAATCCGGATACGAACAAA
AATCAATTTAGCTTACAATTAAATTCCGTCACGCCGGAAGATACAGCGGT
CTATTATTGTGCGCGCGAGGTCACGGGGGATCTCGAAGACGCGTTTGATA
TTTGGGGGCAAGGGACCATGGTGACTGTCAGCTCTGGTGGAGGGGGCAGT
GGAGGTGGGGGATCGGGAGGTGGTGGCAGTGATATTCAAATGATCCAAAG
TCCATCCAGCCTATCCGCATCTGTCGGAGATCGCGTAACGATTACGTGCC
GCGCGAGTCAAACGATTTGGAGCTATCTGAACTGGTACCGGCAACGCCCG
GGCGAAGCGCCGAATCTCTTGATTTACGCGGCGTCCTCATTACAGTCGGG
TGTCCCGAGCCGCTTTAGCGGCCGCGGAAGCGGTACGGATTTTACGTTAA
CCATTAGCAGCCTCCAGGCGGAAGATTTTGCGACGTATTACTGTCAACAG
AGCTATAGCATTCCGCAGACGTTTGGTCAGGGCACGAAATTGGAGATTAA
ACTCGAGACCACCACCCCCGCCCCTAGGCCTCCCACACCTGCCCCCACAA
TCGCCTCCCAGCCTCTCAGCCTGAGGCCTGAAGCTTGCAGGCCCGCTGCC
GGAGGAGCTGTCCATACCAGGGGACTCGACTTCGCCTGCGACATTTACAT
TTGGGCCCCTCTGGCTGGAACCTGCGGAGTCCTGCTGCTGTCCCTGGTGA
TCACACTGTACTGTAAGAGGGGCAGAAAGAAGCTGCTCTACATCTTCAAG
CAGCCCTTTATGAGACCCGTGCAGACAACCCAGGAGGAAGACGGATGCAG
CTGCAGGTTCCCTGAGGAGGAGGAGGGGGGCTGCGAACTGGATATCAGGG
TGAAGTTCAGCAGGAGCGCCGACGCCCCCGCTTATCAACAGGGCCAGAAC
CAGCTGTACAACGAGCTGAACCTCGGCAGAAGAGAGGAGTATGACGTGCT
GGACAAGAGGAGGGGCAGGGACCCTGAGATGGGCGGCAAGCCTAGAAGAA
AGAACCCCCAGGAAGGCCTCTACAACGAACTGCAGAAGGACAAGATGGCC
GAGGCCTACAGCGAGATCGGCATGAAAGGCGAGAGAAGGAGGGGAAAGGG
ACATGACGGCCTGTACCAGGGACTCTCCACAGCCACCAAGGACACCTACG
ATGCCCTGCACATGCAGGCTCTGCCCCCTAGAAGGAAGAGAAGAGGCTCT
GGTACCCCCGATCCTTGGGGAAGCGGCGCTACCAACTTCTCCCTGCTCAA
GCAGGCTGGCGATGTGGAGGAGAACCCCGGCCCCGGATCCATGGCTCTGC
CTGTGACAGCTCTGCTGCTGCCTCTGGCCCTGCTGCTCCATGCTGCTAGA
CCTCAGGTGCAGCTCCAGCAGTCTGGCCCAGGAATGGTCAAGCCTAGCCA
GACCCTGAGCCTGACCTGCGCCATCAGCGGCGACAGCGTGTCCTCTAACA
GCGTCGCCTGGAACTGGATCAGACAGAGCCCCAGCAGAGGCCTGGAATGG
CTGGGCCGGACCTACTACCGGTCCACGTGGTACAACGACTACGCCGTGTC
CATGAAGTCCCGGATCACCATCAACCCCGACACCAACAAGAACCAGTTCT
CCCTGCAGCTGAACAGCGTGACCCCTGAGGACACCGCCGTGTACTACTGC
GCCAGAGAAGTGACCGGCGACCTGGAAGATGCCTTCGACATCTGGGGCCA
GGGCACCATGGTCACCGTGTCTAGCGGAGGCGGCGGAAGCGGTGGAGGCG
GTAGCGGCGGTGGCGGTTCCGACATCCAGATGATCCAGAGCCCTAGCTCC
CTGAGCGCCAGCGTGGGCGACAGAGTGACCATCACCTGTCGGGCCAGCCA
GACCATCTGGTCCTACCTGAATTGGTATCGGCAGCGGCCAGGCGAGGCCC
CTAACCTGCTGATCTATGCCGCCAGCAGCCTGCAGAGCGGCGTGCCAAGC
AGATTCTCTGGCAGAGGCTCCGGCACCGACTTCACCCTGACAATCAGTTC
CCTGCAGGCCGAGGACTTCGCCACCTACTACTGCCAGCAGTCCTACAGCA
TCCCTCAGACCTTCGGCCAGGGGACCAAGCTGGAAATCAAGTCTAGAATC
GAAGTGATGTACCCTCCTCCTTACCTGGACAACGAGAAGTCCAACGGCAC
CATCATCCACGTGAAGGGCAAGCACCTGTGTCCTTCTCCACTGTTCCCCG
GACCTAGCAAGCCTTTCTGGGTGCTCGTTGTTGTTGGCGGCGTGCTGGCC
TGTTACAGCCTGCTGGTTACCGTGGCCTTCATCATCTTTTGGGTCAAGAA
CCGGAAGGCCAAGGCCAAGCCTGTGACAAGAGGTGCTGGTGCTGGCGGCA
GACAGAGAGGCCAGAACAAAGAAAGACCTCCTCCTGTGCCTAATCCTGAC
TACGAGCCCATCCGGAAGGGCCAGAGAGATCTGTATTCTGGCCTGTGAG

In some embodiments, the bicistronic anti-CD22 CAR and anti-CD22 CAR (without 6-His and FLAG tags) provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 96.

### 4. CAR Expression Levels

The present disclosure provides a population of engineered T cells, wherein a plurality of the engineered T cells of the population comprise any chimeric stimulatory receptor (CAR) disclosed herein. The present disclosure also provides a composition comprising a population of T cells, wherein a plurality of the T cells of the population comprise a non-naturally occurring CAR comprising, consisting essentially of, or consisting of: a) a first chimeric antigen receptor (CAR) comprising an antigen recognition domain that binds to a first antigen, a transmembrane domain and a intracellular signaling domain; b) a second CAR comprising an antigen recognition domain that binds to a second antigen, a transmembrane domain and a CD3ε intracellular signaling domain. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the first CAR and the second CAR In some embodiments, each CAR polypeptide is expressed at a copy number of at least 1, 2, 3, 4, 5, 6, 7, 8, 9, 10, 20, 30, 40, 50, 60, 70, 80, 90 or 100 copies per cell. In some embodiments, the nucleic acid encoding the CAR is integrated into the genome at a copy number of at least 1, 2, 3, 4, 5, 6, 7, 8, 9, 10, 20 or 30 copies per cell.

In some embodiments, the ratio of the copy number of CAR1:CAR2 is about 1.1, 2:1, 3.1, 4:1, 5:1, 6:1, 7:1, 8.1, 9:1, 10:1, 1:2, 1:3, 1.4, 1:5, 1:6, 1:7, 1:8, 1.9 or 1.10.

### 5. Antigens

In some embodiments, provided herein are cells (e.g., T cells) expressing a first CAR targeting a first antigen (e.g. anti-CD22) and a second CAR targeting a second antigen (e.g. anti-CD19).

Among the antigens that may be targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may find use in the present method. Exemplary antigens include, but are not limited to, antigenic molecules from infectious agents, glycosylated antigens, TnAntigens, auto-/self-antigens, tumor-/cancer-associated antigens, and tumor neoantigens (Linnemann et al, 2015) In particular aspects, the antigens include those listed in Table 1.

In particular aspects, the antigens for targeting by two or more antigen recognition domains include, but are not limited to CD22 and CD19 (e.g., for B cell malignancies). The sequences for these antigens are known in the art, for example, CD22 (e.g., Accession No. NM_001772.4); CD19 (e.g., Accession No. NC_000023.11).

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in PCT Publication No. WO 99/40188); PRAME; BAGE: RAGE, Lage (also known as NY ESO 1), SAGE, and HAGE or GAGE. These non-limiting examples of tumor antigens are expressed in a wide range of tumor types such as melanoma, lung carcinoma, sarcoma, and bladder carcinoma See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g, src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, protenase3, hTERT, sarcoma translocation breakpoints, EphA2, ML-IAP, EpCAM, ERG (TMPRSS2 ETS fusion gene), NAI 7, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES 1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B 1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g, BK virus and JC virus), adenovirus, Staphylococcus species including Methicillin-resistant Staphylococcus aureus (MRS A), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, pl7, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), E1 and 1E2 (UL123 and UL122), protein products from the cluster of genes from UL28-UL150 (Rykman et al. 2006), envelope glycoprotein B (gB), gH, gN, and ppl50. As would be understood by the skilled person. CMV proteins for use as antigens described herein may be identified in public databases such as GENBANK®, SWISS-PROT® and TREMBL® (see e.g., Bennekov et al. 2004; Loewendorf et al. 2010; Marschall et al. 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof. NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof large protein (L), glycoprotein (G), nucleoprotein (N), phosphoprotein (P), and matrix protein (M) (see, e.g, Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007) As would be understood by the skilled person. Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneunmoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g, B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g, H. influenzae type b outer membrane protein), Helicobacter polypeptides, Klebsiella polypeptides, L-form bacteria polypeptides. Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides. Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g, S. pyogenes M proteins), group B streptococcus (S. agalactiae) polypeptides, Treponema polypeptides, and Yersmia polypeptides (e.g., Y. pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhino sporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides. Parafilaria polypeptides, Paragonimus polypeptides, Parascaris polypeptides. Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Tricho strongylus polypeptides, Trichuris polypeptides, Uncinaria polypeptides, and Wuchereria polypeptides. (e.g, P. falciparum circumsporozoite (PfCSP)), sporozoite surface protein 2 (PfSSP2), carboxyl terminus of liver state antigen 1 (PtLSAl c-term), and exported protein 1 (PfExp-1, Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas, ticks, including hard ticks and soft ticks, flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

### 6. Safety Switch Proteins

Although cellular therapies hold great promise for the treatment of human disease, significant toxicities from the cells themselves or from their transgene products have hampered clinical investigation. In some embodiments described herein, immune effector cells (e.g., T cells) comprising a CAR described herein that have been infused into a mammalian subject, e.g., a human, can be ablated in order to regulate the effect of such immune effector cells should toxicity arise from their use. In some embodiments, the immune cells of the present disclosure may comprise one or more suicide genes.

As used herein, the term “safety switch protein”, “suicide protein” or “kill switch protein” refers to an engineered protein designed to prevent potential toxicity or otherwise adverse effects of a cell therapy in some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch could mediate induction of apoptosis, inhibition of protein synthesis or DNA replication, growth arrest, transcriptional and post-transcriptional genetic regulation and/or antibody-mediated depletion. In some instances, the safety switch protein is activated by an exogenous molecule, e.g., a prodrug, that, when activated, triggers apoptosis and/or cell death of a therapeutic cell.

The term “suicide gene” or “kill switch gene” as used herein is defined as a gene which, upon administration of a prodrug, effects transition of a gene product to a compound which kills its host cell. Examples of suicide gene/prodrug combinations which may be used include, but are not limited to inducible caspase 9 (iCASP9) and rimiducid; RQR8 and rituximab; truncated version of EGFR variant III (EGFRv3) and cetuximab; Herpes Simplex Virus-thymidine kinase (HSV-tk) and ganciclovir, acyclovir, or FIAU; oxidoreductase and cycloheximide, cytosine deaminase and 5-fluorocytosine, thymidine kinase thymidilate kinase (Tdk::Tmk) and AZT, and deoxycytidine kinase and cytosine arabinoside. The E. coli purine nucleoside phosphorylase, a so-called suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene.

Exemplary suicide genes include but are not limited to inducible caspase 9 (or caspase 3 or 7), CD20, CD52, EGFRt, or, thymidine kinase, cytosine deaminase, HER1 and any combination thereof. Further suicide genes known in the art that may be used in the present disclosure include Purine nucleoside phosphorylase (PNP), Cytochrome p450 enzymes (CYP), Carboxypeptidases (CP), Carboxylesterase (CE). Nitroreductase (NTR), Guanine Ribosyltransferase (XGRTP), Glycosidase enzymes, Methionine-a, Y-lyase (MET), and Thymidine phosphorylase (TP).

### 7. T Cell Activity

In some embodiments, a population of genetically engineered T cells as disclosed herein exhibits T cell functions (e.g, effector functions). In some embodiments, the population is cytotoxic to CD22-expressing cells and CD19 expressing cells (e.g., CD22-positive tumor cells, CD22-low tumor cells, CD19 positive tumor cells, CD19 low tumor cells). Effector function of a T cell, for example, may be cytolytic activity or helper activity including the secretion of cytokines. In some embodiments, the population exhibits one or more T cell effector functions at a level that is least 3-4-fold higher than the functions exhibited by a population of T cells not expressing the CAR.

### III. METHODS

Chimeric antigen receptors may be readily inserted into and expressed by immune cells, (e.g, T cells) In certain embodiments, cells (e.g., immune cells such as T cells) are obtained from a donor subject. In some embodiments, the donor subject is human patient afflicted with a cancer or a tumor. In other embodiments, the donor subject is a human patient not afflicted with a cancer or a tumor. In some embodiments, an engineered cell is autologous to a subject. In some embodiments, an engineered cell is allogeneic to a subject.

The cell of the present disclosure may be obtained through any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. In certain embodiments, the cells collected by apheresis are washed to remove the plasma fraction, and placed in an appropriate buffer or media for subsequent processing. In some embodiments, the cells are washed with PBS. As will be appreciated, a washing step can be used, such as by using a semiautomated flowthrough centrifuge, e.g., the Cobe™ 2991 cell processor, the Baxter CytoMate™, or the like. In some embodiments, the washed cells are resuspended in one or more biocompatible buffers, or other saline solution with or without buffer. In certain embodiments, the undesired components of the apheresis sample are removed. Additional methods of isolating T cells for a T cell therapy are disclosed in U.S. Patent Publication No. 2013/0287748, which is herein incorporated by references in its entirety.

In certain embodiments, T cells are isolated from PBMCs by lysing the red blood cells and depleting the monocytes, e.g., by using centrifugation through a PERCOLL™ gradient. In some embodiments, a specific subpopulation of T cells, such as CD4⁺, CD8⁺, CD28⁺, CD45RA⁺, and CD45RO⁺ T cells is further isolated by positive or negative selection techniques known in the art. For example, enrichment of a T cell population by negative selection can be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected can be used. For example, to enrich for CD4⁺ cells by negative selection, a monoclonal antibody cocktail typically includes antibodies to CD8, CD11b, C114, CD16, CD20, and HLA-DR. In certain embodiments, flow cytometry and cell sorting are used to isolate cell populations of interest for use in the present disclosure.

In some embodiments, PBMCs are used directly for genetic modification with the immune cells (such as CARs or TCRs) using methods as described herein. In certain embodiments, after isolating the PBMCs. T lymphocytes are further isolated, and both cytotoxic and helper T lymphocytes are sorted into naive, memory, and effector T cell subpopulations either before or after genetic modification and/or expansion.

In some embodiments, CD8 cells are further sorted into naive, central memory, and effector cells by identifying cell surface antigens that are associated with each of these types of CD8⁺ cells. In some embodiments, the expression of phenotypic markers of central memory T cells includes CCR7, CD3, CD28, CD45RO, CD62L, and CD127 and are negative for granzyme B. In some embodiments, central memory T cells are CD8⁺, CD45RO⁺, and CD62L⁺ T cells. In some embodiments, effector T cells are negative for CCR7, CD28, CD62L, and CD 127 and positive for granzyme B and perforin. In certain embodiments, CD4⁺ T cells are further sorted into subpopulations. For example, CD4⁺ T helper cells can be sorted into naive, central memory, and effector cells by identifying cell populations that have cell surface antigens.

In some embodiments, the immune cells, e.g., T cells, are genetically modified following isolation using known methods, or the immune cells are activated and expanded (or differentiated in the case of progenitors) in vitro prior to being genetically modified. In another embodiment, the immune cells, e.g., T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, e.g., in U.S. Pat. Nos. 6,905,874, 6,867,041; and 6,797,514; and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2 Anti-CD3 and anti-CD28 antibodies attached to the same bead serve as a“surrogate” antigen presenting cell (APC). One example is. The Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

### IV. METHODS OF GENE DELIVERY AND CELL MODIFICATION

One of skill in the art would be well-equipped to construct a vector through standard recombinant techniques (see, for example, Sambrook et al., 2001 and Ausubel et al, 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e g, derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

### 1. Viral Vectors

Viral vectors encoding an antigen receptor, a cytokine and/or an functional effector element may be provided in certain aspects of the methods of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

An engineered virus vector may comprise long terminal repeats (LTRs), a cargo nucleotide sequence, or a cargo cassette. A viral vector-related “cargo cassette” as used herein refers to a nucleotide sequence comprising a left LTR at the 5′ end and a right LTR at the 3′ end, and a nucleotide sequence positioned between the left and right LTRs. The nucleotide sequence flanked by the LTRs is a nucleotide sequence intended for integration into acceptor DNA. A “cargo nucleotide sequence” refers to a nucleotide sequence (e.g., a nucleotide sequence intended for integration into acceptor DNA), flanked by an LTR at each end, wherein the LTRs are heterologous to the nucleotide sequence. A cargo cassette can be artificially engineered.

In some embodiments of the methods of the disclosure, introducing a nucleic acid sequence and/or a genomic editing construct into an immune cell ex vivo, in vivo, in vitro or in situ comprises a viral vector. In some embodiments, the viral vector is a non-integrating non-chromosomal vector. Exemplary non-integrating non-chromosomal vectors include, but are not limited to, adeno-associated virus (AAV), adenovirus, and herpes viruses. In some embodiments, the viral vector is an integrating chromosomal vector. Integrating chromosomal vectors include, but are not limited to, adeno-associated vectors (AAV), Lentiviruses, and gamma-retroviruses.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

A retroviral vector may also be, e.g., a gammaretroviral vector A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., Viruses. 2011 June; 3(6): 677-713.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

In some embodiments of the methods of the disclosure, introducing a nucleic acid sequence and/or a genomic editing construct into an immune cell ex vivo, in vivo, in vitro or in situ comprises a combination of vectors. Exemplary, non-limiting vector combinations include, viral and non-viral vectors, a plurality of non-viral vectors, or a plurality of viral vectors. Exemplary but non-limiting vectors combinations include: a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence, transiently integrates a nucleic acid sequence, produces site-specific integration a nucleic acid sequence, or produces a biased integration of a nucleic acid sequence. In some embodiments, the nucleic acid sequence is a transgene.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence. In some embodiments, the stable chromosomal integration can be a random integration, a site-specific integration, or a biased integration. In some embodiments, the site-specific integration can be non-assisted or assisted. In some embodiments, the assisted site-specific integration is co-delivered with a site-directed nuclease. In some embodiments, the site-directed nuclease comprises a transgene with 5′ and 3′ nucleotide sequence extensions that contain a percentage homology to upstream and downstream regions of the site of genomic integration. In some embodiments, the transgene with homologous nucleotide extensions enable genomic integration by homologous recombination, microhomology-mediated end joining, or nonhomologous end-joining. In some embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

In some embodiments, the site-specific transgene integration occurs at a site that disrupts expression of a target gene. In some embodiments, disruption of target gene expression occurs by site-specific integration at introns, exons, promoters, genetic elements, enhancers, suppressors, start codons, stop codons, and response elements. In some embodiments, exemplary target genes targeted by site-specific integration include but are not limited to any immunosuppressive gene, and genes involved in allo-rejection.

In some embodiments, the site-specific transgene integration occurs at a site that results in enhanced expression of a target gene. In some embodiments, enhancement of target gene expression occurs by site-specific integration at introns, exons, promoters, genetic elements, enhancers, suppressors, start codons, stop codons, and response elements.

### A. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

### (i) Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp-upstream of the start site, although a number of promoters have been shown to contain functional elements downstream of the start site as well. To bring a coding sequence “under the control of a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters, eukaryotic cell promoters, such as, e.g., beta actin promoter, GADPH promoter, metallothionein promoter; and concatenated response element promoters, such as cyclic AMP response element promoters (ere), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is EF1, EF1alpha, MND, CMV IE, dectin-1, dectin-2, human CD11c, F4/80, SM22, RSV, SV40, Ad MLP, beta-actin, MHC class I, MHC class II promoter, U6 promoter or H1 promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter) However, enhancer function is not necessarily restricted to such long distances as they may also function in close proximity to a given promoter.

### (ii) Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription functional effector elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A) or a P2A (e.g. porcine teschovirus-1 2A).

(iii) Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

### B. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### 2. Other Methods of Nucleic Acid Delivery

In addition to viral delivery of the nucleic acids encoding the antigen receptor, the following are additional methods of recombinant gene delivery to a given cell, (e.g. an NK cell) and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading, by liposome mediated transfection and receptor-mediated transfection; by microprojectile bombardment, by agitation with silicon carbide fibers; by Agrobacterium-mediated transformation, by desiccation/inhibition-mediated DNA uptake, and any combination of such methods. Through the application of techniques such as these, organelle(s), cell(s), tissue(s) or organism(s) may be stably or transiently transformed.

### A. Transposition Based Methods of Modification

Generally, the gene transfer system can include a transposon-based or a viral-based integration system.

In some embodiments, the gene transfer system comprises a transposon system. DNA transposons can translocate via a non-replicative “cut-and-paste” mechanism. This mechanism requires recognition of the two inverse terminal repeats (ITRs) by a catalytic enzyme, i.e., transposase, which can cleave its target and consequently release the DNA transposon from its donor template. Upon excision, the DNA transposons may subsequently integrate into the acceptor DNA that is cleaved by the same transposase. In some of their natural configurations, DNA transposons are flanked by two ITRs and may contain a gene encoding a transposase that catalyzes transposition.

Transposon systems offer many advantages for nucleic acid integration, e.g., as compared to viral vectors. For example, transposons can carry larger cargos, which can be advantageous for delivering one or more of the CARs, functional effector elements, and/or cytokines disclosed herein, to an immune cell (e.g., an NK cell) Further, transposons may comprise, for example, CRISPR tools (e.g., along with cargo), and thereby allow multiplex engineering of a cell.

A transposon system comprises (i) a plasmid backbone with inverse terminal repeats (ITRs) and (ii) a transposase enzyme that recognizes the ITRs. The term “inverse terminal repeats,” “inverted terminal repeats”, or “ITRs”, as used interchangeably herein, refers to short sequence repeats flanking the transposase gene in a natural transposon, or flanking a cargo polynucleotide sequence in an artificially engineered transposon. Two inverted terminal repeats are generally required for the mobilization of the transposon in the presence of a corresponding transposase. Inverted repeats as described herein may contain one or more direct repeat (DR) sequences. These DR sequences usually are embedded in the terminal inverted repeats (ITRs) of the elements. The compositions and methods of the present disclosure comprise, in various embodiments, one or more artificially engineered transposons. An engineered transposon may comprise ITRs, a cargo nucleotide sequence, or a cargo cassette. A transposon-related “cargo cassette” as used herein refers to a nucleotide sequence comprising a left ITR at the 5′ end and a right ITR at the 3′ end, and a nucleotide sequence positioned between the left and right ITRs. The nucleotide sequence flanked by the ITRs is a nucleotide sequence intended for integration into acceptor DNA. The cargo cassette can, in some embodiments, be comprised in a vector, such as plasmid. A “cargo nucleotide sequence” refers to a nucleotide sequence (e.g., a nucleotide sequence intended for integration into acceptor DNA), flanked by an ITR at each end, wherein the ITRs are heterologous to the nucleotide sequence. A cargo cassette can be artificially engineered.

### Transposons and Transposase

Exemplary transposon systems for use as described in the disclosure include, but are not limited to, piggyBac, hyperactive piggyBac, Sleeping Beauty (SB), hyperactive Sleeping Beauty (SB100x), SB11, SB110, Tn7, TcBuster, hyperactive TcBuster, Frog Prince, IS5, TnlO, Tn903, SPIN, hAT, Hermes, Hobo, AeBuster1, AeBuster2, AeBuster3, BtBuster1, BtBuster2, CfBuster1, CfBuster2, Tol2, mini-Tol2, Tc3, Mos1, MuA, Himar I, Helitron, and engineered versions of transposase family enzymes (Zhang et al. (2009) PLoS Genet. 5:e 1000689; Wilson et al. (2007) J. Microbiol. Methods 71: 332-5, the entire contents of which are incorporated by reference herein). Exemplary transposons also include the transposons of the hAT transposon superfamily described in Arensburger et al. (2011) Genetics 188(1): 45-57, the entire contents of which are incorporated by reference herein) or a SPACE INVADERS (SPIN) transposon (see, e.g., Pace et al. (2008) Proc. Natl. Acad. Sci. USA. 2008; 105(44):17023-17028, the entire contents of which are incorporated by reference herein).

In some embodiments, the gene transfer system can be delivered to the cell encoded in DNA, encoded in mRNA, as a protein, or as a nucleoprotein complex. Alternatively, the gene transfer system can be integrated into the genome of a host cell using, for example, a retro-transposon, random plasmid integration, recombinase-mediated integration, homologous recombination mediated integration, or non-homologous end joining mediated integration More examples of transposition systems that can be used with certain embodiments of the compositions and methods provided herein include Staphylococcus aureus Tn552 (Colegio et al. J. BacterioL, 183: 2384-8, 2001; Kirby C et al, Mol. Microbiol, 43: 173-86, 2002), Tyl (Devine & Boeke, Nucleic Acids Res. 22: 3765-72, 1994 and International Publication WO 95/23875), Transposon Tn7 (Craig, N L, Science. 271: 1512, 1996; Craig, N L, Review in: Curr Top Microbiol Immunol, 204:27-48, 1996), Tn/O and IS10 (Kleckner N, et al, Curr Top Microbiol Immunol, 204:49-82, 1996), Mariner transposase (Lampe D J, et al, EMBO J., 15: 5470-9, 1996), Tel (Plasterk R H, Curr. Topics Microbiol. Immunol, 204: 125-43, 1996), P Element (Gloor, G B, Methods Mol. Biol, 260: 97-114, 2004), Tn3 (Ichikawa & Ohtsubo, J Biol. Chem. 265: 18829-32, 1990), bacterial insertion sequences (Ohtsubo & Sekine, Curr. Top. Microbiol. Immunol. 204: 1-26, 1996), retroviruses (Brown, et al, Proc Natl Acad Sci USA, 86:2525-9, 1989), and retrotransposon of yeast (Boeke & Corces, Annu Rev Microbiol. 43:403-34, 1989). The entire contents of each of the foregoing references are incorporated by reference herein.

Transposition efficiency can be measured by the percent of successful transposition events occurring in a population of host cells normalized by the amount of transposon and transposase introduced into the population of host cells. In many instances, when the transposition efficiency of two or more transposases is compared, the same transposon construct is paired with each of the two or more transposases for transfection of the host cells under same or similar transfection conditions. The amount of transposition events in the host cells can be examined by various approaches. For example, the transposon construct may be designed to contain a reporter gene positioned between the inverted repeats, and transfected cells positive for the reporter gene can be counted as the cells where successful transposition events occurs, which can give an estimate of the amount of the transposition events Another non-limiting example includes sequencing of the host cell genome to examine the insertion of the cassette cargo of the transposon in some embodiments, when the transposition efficiency of two or more different transposons is compared, the same transposase can be paired with each of the different transposons for transfection of the host cells under same or similar transfection conditions. Similar approaches to the above, and other methods commonly known to one skilled in the art, may also be implemented for the comparison of transposition efficiency.

### Polynucleotides Encoding the Transposase System

One aspect of the present disclosure provides a polynucleotide comprising a nucleotide sequence that encodes for a transposase described herein. In some embodiments, the polynucleotide further comprises a nucleotide sequence of a transposon (e.g., an engineered transposon) recognizable by the transposase. In some embodiments, the polynucleotide is comprised in an expression vector. In some embodiments, the expression vector is a DNA plasmid. In some embodiments, the expression vector is a mini-circle vector. In some embodiments, the expression vector is a nanoplasmid.

The term “mini-circle vector” as used herein can refer to a small circular plasmid derivative that is free of most, if not all, prokaryotic vector parts (e.g., control sequences or non-functional sequences of prokaryotic origin).

For genome editing applications with transposons, in some embodiments, it may be desirable to design a transposon for use in a binary system based on two distinct plasmids, whereby the nucleic acid sequence encoding for the transposase is physically separated from the transposon nucleic acid sequence containing the gene of interest flanked by the inverted repeats. Co-delivery of the transposon and transposase-encoding plasmids into the target cells enables transposition via a conventional cut-and-paste mechanism. In some other embodiments, a transposon based system as described herein may comprise a polynucleotide comprising both a nucleic acid sequence encoding a transposase as described herein, and a nucleic acid sequence of a transposon as described herein, i.e., wherein the nucleic acid encoding for the transposase and the transposon nucleic acid are present in the same plasmid.

One of the limitations of application of plasmid vectors is that transgene expression duration from plasmid vectors is reduced due to promoter inactivation mediated by the bacterial region (i.e., the region encoding the bacterial replication origin and selectable marker) of the vector (Chen et al., 2004. Gene Ther 11:856-864; Suzuki et al., 2006. J Virol 80: 293-3300) This results in short duration transgene expression. A strategy to improve transgene expression duration is to remove the bacterial region of the plasmid. For example, minicircle vectors have been developed which do not contain a bacterial region. Removal of the bacterial region in minicircle vectors improved transgene expression duration (Chen et al., 2004). In minicircle vectors, the eukaryotic region polyadenylation signal is covalently linked to the eukaryotic region promoter through a short spacer typically less than 200 bp comprised of the recombined attachment sites. This linkage (spacer region) can tolerate a much longer spacer sequence since while long spacers >1 kb in length resulted in transgene expression silencing in vivo, shorter spacers <500 bp exhibited similar transgene expression patterns to conventional minicircle DNA vectors (Lu et al. 2012. Mol Ther. 20.2111-9).

In some embodiments, a vector useful in various aspects of the disclosure is a nanoplasmid vector. The term “nanoplasmid vector” as used herein, refers to a vector combining an RNA selectable marker with a R6K, ColE2 or ColE2 related replication origin. Nanoplasmid vectors can be selected from the nanoplasmid vectors disclosed in any of International PCT Publication No. WO2014/035457, International PCT Publication No. WO2014/077866, and International PCT Publication No. WO2019/183248, each of which is incorporated in its entirety herein by reference. For example, International PCT Publication No. WO2014/035457 discloses minimalized nanoplasmid vectors that utilize RNA-OUT antibiotic-free selection and replace the large 1000 bp pUC replication origin with a novel, 300 bp, R6K origin, which result in improved expression from the plasmid. Reduction of the spacer region linking the 5′ and 3′ ends of the transgene expression cassette to <500 bp with R6K origin-RNA-OUT backbones improved expression duration to that of conventional minicircle DNA vectors. The 1.1 kb pFAR4 vector pUC-origin tRNA antibiotic free selection spacer has improved expression duration compared to a 2.2 kb pUC origin-kanR antibiotic selection marker spacer region (Quiviger et al., 2014. Gene Therapy 21: 1001-1007). This indicates that improved expression duration can be obtained with some bacterial regions up to 1.1 kb. Expression level improvement compared to plasmid vectors is also observed with some spacer regions<1.1 kb. For example, pVAX1 derivatives with the 2 kb bacterial backbone reduced to 1.2, 1.1 or 0.7 kb show >2-fold improved expression compared to the parent pVAX1 vector. NTC8685 derivatives with the 1.5 kb bacterial backbone reduced to 0.9 kb, 466 bp or 281 bp (nanoplasmid vectors) show >2-fold improved expression compared to the parent NTC8685 vector.

In some embodiments, the nanoplasmid vector is useful for viral and non-viral gene therapy, viral and non-viral cell therapy, and more particularly, for improving viral and non-viral vector manufacturing yield and quality, for reducing transfection associated toxicity, for improving transposition from non-viral transposon vectors, for improving packaging titers from viral vectors, for improving expression of viral and non-viral vector encoded transgenes, and for eliminating antibiotic resistance marker gene transfer by viral and non-viral vectors, as described in International PCT Publication No. WO2019/183248, which is incorporated in its entirety herein by reference.

In some embodiments, the nanoplasmid vector comprises modifications that improve the replication of the vector. In some embodiments, the nanoplasmid vector utilizes a Pol III-dependent origin of replication to replicate. In some embodiments, the nanoplasmid vector utilizes a Pol I-dependent origin of replication to replicate. In some embodiments, the nanoplasmid vector comprises an antibiotic selectable marker. In some embodiments, the nanoplasmid vector does not comprise an antibiotic selectable marker. In some embodiments, the nanoplasmid vector comprises an RNA selectable marker.

### B. Other Methods of Modification

In some embodiments of the methods of the disclosure, a modified immune cell of the disclosure may be produced by introducing a transgene into an immune cell of the disclosure. The introducing step may comprise delivery of a nucleic acid sequence and/or a genomic editing construct via a non-transposition delivery system.

In some embodiments of the methods of the disclosure, introducing a nucleic acid sequence and/or a genomic editing construct into an immune cell ex vivo, in vivo, in vitro or in situ comprises one or more of topical delivery, adsorption, absorption, electroporation, spin-fection, co-culture, transfection, mechanical delivery, sonic delivery, vibrational delivery, magnetofection or by nanoparticle-mediated delivery. In some embodiments of the methods of the disclosure, introducing a nucleic acid sequence and/or a genomic editing construct into an immune cell ex vivo, in vivo, in vitro or in situ comprises liposomal transfection, calcium phosphate transfection, fugene transfection, and dendrimer-mediated transfection. In some embodiments of the methods of the disclosure, introducing a nucleic acid sequence and/or a genomic editing construct into an immune cell ex vivo, in vivo, in vitro or in situ by mechanical transfection comprises cell squeezing, cell bombardment, or gene gun techniques. In some embodiments of the methods of the disclosure, introducing a nucleic acid sequence and/or a genomic editing construct into an immune cell ex vivo, in vivo, in vitro or in situ by nanoparticle-mediated transfection comprises liposomal delivery, delivery by micelles, and delivery by polymerosomes.

In some embodiments of the methods of the disclosure, introducing a nucleic acid sequence and/or a genomic editing construct into an immune cell ex vivo, in vivo, in vitro or in situ comprises a non-viral vector. In some embodiments, the non-viral vector comprises a nucleic acid. In some embodiments, the non-viral vector comprises plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In some embodiments, the non-viral vector comprises a transposon of the disclosure.

In some embodiments of the methods of the disclosure, enzymes may be used to create strand breaks in the host genome to facilitate delivery or integration of the transgene. In some embodiments, enzymes create single-strand breaks. In some embodiments, enzymes create double-strand breaks. In some embodiments, examples of break-inducing enzymes include but are not limited to, transposases, integrases, endonucleases, meganucleases, megaTALs, CRISPR-Cas9, CRISPR-CasX, transcription activator-like effector nucleases (TALEN) or zinc finger nucleases (ZFN). In some embodiments, break-inducing enzymes can be delivered to the cell encoded in DNA, encoded in mRNA, as a protein, as a nucleoprotein complex with a guide RNA (gRNA).

In some embodiments of the methods of the disclosure, the site-specific transgene integration is controlled by a vector-mediated integration site bias. In some embodiments vector-mediated integration site bias is controlled by the chosen lentiviral vector. In some embodiments vector-mediated integration site bias is controlled by the chosen gamma-retroviral vector.

In some embodiments of the methods of the disclosure, the site-specific transgene integration site is a non-stable chromosomal insertion. In some embodiments, the integrated transgene may become silenced, removed, excised, or further modified.

In some embodiments of the methods of the disclosure, the genome modification is a non-stable integration of a transgene. In some embodiments, the non-stable integration can be a transient non-chromosomal integration, a semi-stable non chromosomal integration, a semi-persistent non-chromosomal insertion, or a non-stable chromosomal insertion. In some embodiments, the transient non-chromosomal insertion can be epi-chromosomal or cytoplasmic.

In some embodiments, the transient non-chromosomal insertion of a transgene does not integrate into a chromosome and the modified genetic material is not replicated during cell division.

In some embodiments of the methods of the disclosure, the genome modification is a semi-stable or persistent non-chromosomal integration of a transgene. In some embodiments, a DNA vector encodes a Scaffold/matrix attachment region (S-MAR) module that binds to nuclear matrix proteins for episomal retention of a non-viral vector allowing for autonomous replication in the nucleus of dividing cells.

In some embodiments of the methods of the disclosure, the genome modification is a non-stable chromosomal integration of a transgene. In some embodiments, the integrated transgene may become silenced, removed, excised, or further modified.

In some embodiments of the methods of the disclosure, the modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. In some embodiments, the modification to the genome by transgene insertion can occur via CRISPR-Cas9, CRISPR-CasX, TALEN or ZFNs.

### C. Nanoparticle Delivery

Poly(histidine) (i.e., poly(L-histidine)), is a pH-sensitive polymer due to the imidazole ring providing an electron lone pair on the unsaturated nitrogen. That is, poly(histidine) has amphoteric properties through protonation-deprotonation. The various embodiments enable intracellular delivery of gene editing tools by complexing with poly(histidine)-based micelles. In particular, the various embodiments provide triblock copolymers made of a hydrophilic block, a hydrophobic block, and a charged block. In some embodiments, the hydrophilic block may be poly(ethylene oxide) (PEO), and the charged block may be poly(L-histidine). An example tri-block copolymer that may be used in various embodiments is a PEO-b-PLA-b-PHIS, with variable numbers of repeating units in each block varying by design. The gene editing tools may be various molecules that are recognized as capable of modifying, repairing, adding and/or silencing genes in various cells. The correct and efficient repair of double-strand breaks (DSBs) in DNA is critical to maintaining genome stability in cells. Structural damage to DNA may occur randomly and unpredictably in the genome due to any of a number of intracellular factors (e.g., nucleases, reactive oxygen species, etc.) as well as external forces (e.g., ionizing radiation, ultraviolet (UV) radiation, etc.). In particular, correct and efficient repair of double-strand breaks (DSBs) in DNA is critical to maintaining genome stability. Accordingly, cells naturally possess a number of DNA repair mechanisms, which can be leveraged to alter DNA sequences through controlled DSBs at specific sites. Genetic modification tools may therefore be composed of programmable, sequence-specific DNA-binding modules associated with a nonspecific DNA nuclease, introducing DSBs into the genome. For example, CRISPR, mostly found in bacteria, are loci containing short direct repeats, and are part of the acquired prokaryotic immune system, conferring resistance to exogenous sequences such as plasmids and phages. RNA-guided endonucleases are programmable genetic engineering tools that are adapted from the CRISPR/CRISPR-associated protein 9 (Cas9) system, which is a component of prokaryotic innate immunity.

Diblock copolymers that may be used as intermediates for making triblock copolymers of the embodiment micelles may have hydrophilic biocompatible poly(ethylene oxide) (PEO), which is chemically synonymous with PEG, coupled to various hydrophobic aliphatic poly(anhydrides), poly(nucleic acids), poly(esters), poly(ortho esters), poly(peptides), poly(phosphazenes) and poly(saccharides), including but not limited by poly(lactide) (PLA), poly(glycolide) (PLGA), poly(lactic-co-glycolic acid) (PLGA), poly(ε-caprolactone) (PCL), and poly(trimethylene carbonate) (PTMC). Polymeric micelles comprised of 1000% PEGylated surfaces possess improved in vitro chemical stability, augmented in vivo bioavailability, and prolonged blood circulatory half-lives. For example, aliphatic polyesters, constituting the polymeric micelle's membrane portions, are degraded by hydrolysis of their ester linkages in physiological conditions such as in the human body. Because of their biodegradable nature, aliphatic polyesters have received a great deal of attention for use as implantable biomaterials in drug delivery devices, bioresorbable sutures, adhesion barriers, and as scaffolds for injury repair via tissue engineering.

In various embodiments, molecules required for gene editing (i.e., gene editing tools) may be delivered to cells using one or more micelle formed from self-assembled triblock copolymers containing poly(histidine). The term “gene editing” as used herein refers to the insertion, deletion or replacement of nucleic acids in genomic DNA so as to add, disrupt or modify the function of the product that is encoded by a gene. Various gene editing systems require, at a minimum, the introduction of a cutting enzyme (e.g., a nuclease or recombinase) that cuts genomic DNA to disrupt or activate gene function.

Further, in gene editing systems that involve inserting new or existing nucleotides/nucleic acids, insertion tools (e.g. DNA template vectors, transposable elements (transposons or retrotransposons) must be delivered to the cell in addition to the cutting enzyme (e.g. a nuclease, recombinase, integrase or transposase). Examples of such insertion tools for a recombinase may include a DNA vector. Other gene editing systems require the delivery of an integrase along with an insertion vector, a transposase along with a transposon/retrotransposon, etc. In some embodiments, an example recombinase that may be used as a cutting enzyme is the CRE recombinase. In various embodiments, example integrases that may be used in insertion tools include viral based enzymes taken from any of a number of viruses including, but not limited to, AAV, gamma retrovirus, and lentivirus. Example transposons/retrotransposons that may be used in insertion tools include, but are not limited to, the piggyBac® transposon, Sleeping Beauty transposon, TcBuster transposon and the L1 retrotransposon.

In certain embodiments of the methods of the disclosure, the transgene is delivered in vivo. In certain embodiments of the methods of the disclosure, in vivo transgene delivery can occur by: topical delivery, adsorption, absorption, electroporation, spin-fection, co-culture, transfection, mechanical delivery, sonic delivery, vibrational delivery, magnetofection or by nanoparticle-mediated delivery. In certain embodiments of the methods of the disclosure, in vivo transgene delivery by transfection can occur by liposomal transfection, calcium phosphate transfection, fugene transfection, and dendrimer-mediated transfection. In certain embodiments of the methods of the disclosure, in vivo mechanical transgene delivery can occur by cell squeezing, bombardment, and gene gun. In certain embodiments of the methods of the disclosure, in vivo nanoparticle-mediated transgene delivery can occur by liposomal delivery, delivery by micelles, and delivery by polymerosomes. In various embodiments, nucleases that may be used as cutting enzymes include, but are not limited to, Cas9, transcription activator-like effector nucleases (TALENs) and zinc finger nucleases.

In various embodiments, the gene editing systems described herein, particularly proteins and/or nucleic acids, may be complexed with nanoparticles that are poly(histidine)-based micelles. In particular, at certain pHs, poly(histidine)-containing triblock copolymers may assemble into a micelle with positively charged poly(histidine) units on the surface, thereby enabling complexing with the negatively-charged gene editing molecule(s). Using these nanoparticles to bind and release proteins and/or nucleic acids in a pH-dependent manner may provide an efficient and selective mechanism to perform a desired gene modification. In particular, this micelle-based delivery system provides substantial flexibility with respect to the charged materials, as well as a large payload capacity, and targeted release of the nanoparticle payload. In one example, site-specific cleavage of the double stranded DNA may be enabled by delivery of a nuclease using the poly(histidine)-based micelles.

The various embodiments enable intracellular delivery of gene editing tools by complexing with poly(histidine)-based micelles. In particular, the various embodiments provide triblock copolymers made of a hydrophilic block, a hydrophobic block, and a charged block. In some embodiments, the hydrophilic block may be poly(ethylene oxide) (PEO), and the charged block may be poly(L-histidine). An example tri-block copolymer that may be used in various embodiments is a PEO-b-PLA-b-PHIS, with variable numbers of repeating units in each block varying by design. Without wishing to be bound by a particular theory, it is believed that believed that in the micelles that are formed by the various embodiment triblock copolymers, the hydrophobic blocks aggregate to form a core, leaving the hydrophilic blocks and poly(histidine) blocks on the ends to form one or more surrounding layer.

In certain embodiments of the methods of the disclosure, non-viral vectors are used for transgene delivery. In certain embodiments, the non-viral vector is a nucleic acid. In certain embodiments, the nucleic acid non-viral vector is plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In certain embodiments, the non-viral vector is a transposon. In certain embodiments, the transposon is TcBuster.

In certain embodiments of the methods of the disclosure, transgene delivery can occur via viral vector. In certain embodiments, the viral vector is a non-integrating non-chromosomal vectors. Non-integrating non-chromosomal vectors can include adeno-associated virus (AAV), adenovirus, and herpes viruses. In certain embodiments, the viral vector is an integrating chromosomal vectors. Integrating chromosomal vectors can include adeno-associated vectors (AAV), Lentiviruses, and gamma-retroviruses.

In certain embodiments of the methods of the disclosure, transgene delivery can occur by a combination of vectors. Exemplary but non-limiting vector combinations can include: viral plus non-viral vectors, more than one non-viral vector, or more than one viral vector. Exemplary but non-limiting vectors combinations can include: DNA-derived plus RNA-derived vectors, RNA plus reverse transcriptase, a transposon and a transposase, a non-viral vectors plus an endonuclease, and a viral vector plus an endonuclease.

In certain embodiments of the methods of the disclosure, the genome modification can be a stable integration of a transgene, a transient integration of a transgene, a site-specific integration of a transgene, or a biased integration of a transgene.

In certain embodiments of the methods of the disclosure, the genome modification can be a stable chromosomal integration of a transgene. In certain embodiments, the stable chromosomal integration can be a random integration, a site-specific integration, or a biased integration. In certain embodiments, the site-specific integration can be non-assisted or assisted. In certain embodiments, the assisted site-specific integration is co-delivered with a site-directed nuclease. In certain embodiments, the site-directed nuclease comprises a transgene with 5′ and 3′ nucleotide sequence extensions that contain homology to upstream and downstream regions of the site of genomic integration. In certain embodiments, the transgene with homologous nucleotide extensions enable genomic integration by homologous recombination, microhomology-mediated end joining, or nonhomologous end-joining. In certain embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

In certain embodiments, the site-specific transgene integration occurs at a site that disrupts expression of a target gene. In certain embodiments, disruption of target gene expression occurs by site-specific integration at introns, exons, promoters, genetic elements, enhancers, suppressors, start codons, stop codons, and response elements. In certain embodiments, exemplary target genes targeted by site-specific integration include but are not limited to any immunosuppressive gene, and genes involved in allo-rejection.

In certain embodiments, the site-specific transgene integration occurs at a site that results in enhanced expression of a target gene. In certain embodiments, enhancement of target gene expression occurs by site-specific integration at introns, exons, promoters, genetic elements, enhancers, suppressors, start codons, stop codons, and response elements.

In certain embodiments of the methods of the disclosure, enzymes may be used to create strand breaks in the host genome to facilitate delivery or integration of the transgene. In certain embodiments, enzymes create single-strand breaks. In certain embodiments, enzymes create double-strand breaks. In certain embodiments, examples of break-inducing enzymes include but are not limited to: transposases, integrases, endonucleases, meganucleases, megaTALs, CRISPR-Cas9, CRISPR-CasX, transcription activator-like effector nucleases (TALEN) and zinc finger nucleases (ZFN). In certain embodiments, break-inducing enzymes can be delivered to the cell encoded in DNA, encoded in mRNA, as a protein, as a nucleoprotein complex with a guide RNA (gRNA).

In certain embodiments of the methods of the disclosure, the site-specific transgene integration is controlled by a vector-mediated integration site bias. In certain embodiments vector-mediated integration site bias is controlled by the chosen lentiviral vector. In certain embodiments vector-mediated integration site bias is controlled by the chosen gamma-retroviral vector.

In certain embodiments of the methods of the disclosure, the site-specific transgene integration site is a non-stable chromosomal insertion. In certain embodiments, the integrated transgene may become silenced, removed, excised, or further modified. In certain embodiments of the methods of the disclosure, the genome modification is a non-stable integration of a transgene. In certain embodiments, the non-stable integration can be a transient non-chromosomal integration, a semi-stable non chromosomal integration, a semi-persistent non-chromosomal insertion, or a non-stable chromosomal insertion. In certain embodiments, the transient non-chromosomal insertion can be epi-chromosomal or cytoplasmic. In certain embodiments, the transient non-chromosomal insertion of a transgene does not integrate into a chromosome and the modified genetic material is not replicated during cell division.

In certain embodiments of the methods of the disclosure, the genome modification is a semi-stable or persistent non-chromosomal integration of a transgene. In certain embodiments, a DNA vector encodes a Scaffold/matrix attachment region (S-MAR) module that binds to nuclear matrix proteins for episomal retention of a non-viral vector allowing for autonomous replication in the nucleus of dividing cells.

In certain embodiments of the methods of the disclosure, the genome modification is a non-stable chromosomal integration of a transgene. In certain embodiments, the integrated transgene may become silenced, removed, excised, or further modified.

In certain embodiments of the methods of the disclosure, the modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. In certain embodiments, the modification to the genome by transgene insertion can occur via CRISPR-Cas9, CRISPR-CasX, TALEN or ZFNs.

In certain embodiments of the methods of the disclosure, a cell with an in vivo or ex vivo genomic modification can be a germline cell or a somatic cell. In certain embodiments the modified cell can be a human, non-human, mammalian, rat, mouse, or dog cell. In certain embodiments, the modified cell can be differentiated, undifferentiated, or immortalized. In certain embodiments, the modified undifferentiated cell can be a stem cell. In certain embodiments, the modified cell can be differentiated, undifferentiated, or immortalized. In certain embodiments, the modified undifferentiated cell can be an induced pluripotent stem cell. In certain embodiments, the modified cell can be a T cell, a hematopoietic stem cell, a natural killer cell, a macrophage, a dendritic cell, a monocyte, a megakaryocyte, or an osteoclast. In certain embodiments, the modified cell can be modified while the cell is quiescent, in an activated state, resting, in interphase, in prophase, in metaphase, in anaphase, or in telophase. In certain embodiments, the modified cell can be fresh, cryopreserved, bulk, sorted into sub-populations, from whole blood, from leukapheresis, or from an immortalized cell line.

### B. ZFPs and ZFNs

In some embodiments, the DNA-targeting molecule includes a DNA-binding protein such as one or more zinc finger protein (ZFP) or transcription activator-like protein (TAL), fused to an effector protein such as an endonuclease Examples include ZFNs, TALEs, and TALENs.

In some embodiments, the DNA-targeting molecule comprises one or more zinc-finger proteins (ZFPs) or domains thereof that bind to DNA in a sequence-specific manner A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type liS restriction enzyme and one or more zinc finger binding domains, which may or may not be engineered. In some embodiments, the cleavage domain is from the Type liS restriction endonuclease Fok I. Fok I generally catalyzes double-stranded cleavage of DNA, at 9 nucleotides from its recognition site on one strand and 13 nucleotides from its recognition site on the other.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al, Trends in Biotechnology, 2013, 31(7), 397-405) In some embodiments, commercially available zinc fingers are used or are custom designed. (See, for example, Sigma-Aldrich catalog numbers CSTZFND, CSTZFN, CTil-IKT, and PZD0020).

### C. TALs, TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U. S. Patent Publication No 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NO binds to T and non-canonical (atypical) RVDs are also known. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ) Generally, NHEJ is an imperfect repair process that often results in changes to the DNA sequence at the site of the cleavage. In some aspects, repair mechanisms involve rejoining of what remains of the two DNA ends through direct re-ligation or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. (Gaj et al., 2013). A library of TALENs targeting 18,740 human protein-coding genes has been constructed (Kim et at, 2013) Custom-designed TALE arrays are commercially available through Cellectis Bioresearch (Paris. France), Transposagen Biopharmaceuticals (Lexington, KY, USA), and Life Technologies (Grand Island, NY, USA). Specifically, TALENs that target CD38 are commercially available (See Gencopoeia, catalog numbers HTN222870-1. HTN222870-2, and HTN222870-3). Exemplary molecules are described, e.g., in U.S. Patent Publication Nos. US 2014/0120622, and 2013/0315884.

In some embodiments the TALEN s are introduced as trans genes encoded by one or more plasmid vectors. In some aspects, the plasmid vector can contain a selection marker which provides for identification and/or selection of cells which received said vector.

### D. Meganucleases and MegaTALs

In certain embodiments, the nuclease comprises a meganuclease (homing endonuclease) or a portion thereof that exhibits cleavage activity. In some embodiments, a “meganuclease,” also referred to as a “homing endonuclease,” refers to an endodeoxyribonuclease characterized by a large recognition site (double stranded DNA sequences of about 12 to about 40 base pairs). Naturally-occurring meganucleases recognize 15-40 base-pair cleavage sites and are commonly grouped into four families' the LAGLIDADG family, the GIY-YIG family, the His-Cyst box family and the HNH family Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. No. 5,420,032: U.S. Pat. No. 6,833,252; Belfort et al. (1997) Nucleic Acids Res. 25: 3379-3388: Dujon et al. (1989) Gene 82: 115-118: Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol Biol. 280: 345-353 and the New England Biolabs catalogue.

DNA-binding domains from naturally-occurring meganucleases, primarily from the LAGLIDADG family, have been used to promote site-specific genome modification in plants, yeast, Drosophila, mammalian cells and mice, but this approach has been limited to the modification of either homologous genes that conserve the meganuclease recognition sequence (Monet et al (1999). Biochem. Biophysics. Res. Common 255: 88-93) or to pre-engineered genomes into which a recognition sequence has been introduced (Route et al (1994). Mol. Cell. Biol. 14: 8096-106, Chilton et al. (2003), Plant Physiology. 133: 956-65; Puchta et al. (1996), Proc Natl. Acad. Sci USA 93: 5055-60, Rong et al. (2002), Genes Dev. 16: 1568-81; Gouble et al. (2006), J. Gene Med. 8(5):616-622) Accordingly, attempts have been made to engineer meganucleases to exhibit novel binding specificity at medically or biotechnologically relevant sites (Porteus et al. (2005), Nat. Biotechnol. 23: 967-73; Sussman et al. (2004), J. Mol. Biol. 342: 31-41; Epinat et al. (2003), Nucleic Acids Res. 31: 2952-62; Chevalier et al. (2002) Molec. Cell 10.895-905, Epinat et al. (2003) Nucleic Acids Res. 31.2952-2962, Ashworth et al. (2006) Nature 441:656-659; Paques et al. (2007) Current Gene Therapy 7:49-66; U.S. Patent Publication Nos. 20070117128; 20060206949, 20060153826; 20060078552; and 20040002092). In addition, naturally-occurring or engineered DNA-binding domains from meganucleases can be operably linked with a cleavage domain from a heterologous nuclease (e.g., FokI) and/or cleavage domains from meganucleases can be operably linked with a heterologous DNA-binding domain (e.g., ZFP or TALE).

In any of the nucleases described herein, the nuclease can comprise an engineered TALE DNA-binding domain and a nuclease domain (e.g., endonuclease and/or meganuclease domain), also referred to as TALENs. Methods and compositions for engineering these TALEN proteins for robust, site specific interaction with the target sequence of the user's choosing have been published (see U.S. Pat. No. 8,586,526). In some embodiments, the TALEN comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al., (2013) Nucl Acid Res: 1-13, doi: 10.1093/nar/gkt1224). In addition, the nuclease domain may also exhibit DNA-binding functionality.

E. RGENs (CRISPR/Cas systems)

In some embodiments, the alteration is carried out using one or more DNA-binding nucleic acids, such as alteration via an RNA-guided endonuclease (RGEN) For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR, complex, such as at least 50%, 60%, 70%, 80%, 90%, 95% or 99% of sequence complementarity along the length of the tracr mate sequence when optimally aligned.

The components of a CRISPR system can be implemented in any suitable manner, meaning that the components of such systems including the RNA-guided nuclease (e.g., Cas enzyme) and gRNA can be delivered, formulated or administered in any suitable form to the cells. For example, the RNA-guided nuclease may be delivered to a cell complexed with a gRNA (e.g., as a ribonucleoprotein (RNP) complex), the RNA-guided nuclease may be delivered to a cell separate (e.g., uncomplexed) to a gRNA, the RNA-guided nuclease may be delivered to a cell as a polynucleotide (e.g., DNA or RNA) encoding the nuclease that is separate from a gRNA, or both the RNA-guided nuclease and the gRNA molecule may be delivered as polynucleotides encoding each component.

One or more vectors driving expression of one or more elements of the CRISPR system can be introduced into the cell such that expression of the elements of the CRISPR system direct formation of the CRISPR complex at one or more target sites Components can also be delivered to cells as ribonucleoprotein complexes, proteins, DNA, and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. In addition, a nucleic acid encoding the endonuclease (e.g., a Cas enzyme such as Cas8 or Cas9) may be delivered with gRNAs. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csnl and Csxl2), Cas10, CasX, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csxl7, Csxl4, CsxlO, Csx16, CsaX, Csx3, Csx1, Csx15, Csf1, Csf2, Csf3, Csf4, homologs thereof, or modified versions thereof. These enzymes are known; for example, the amino acid sequence of S pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR, enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand) In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.). SOAP (available at soap.genomics.org.cn), and Maq (available at maq.sourceforge.net).

The CRISPR enzyme may be part of a fusion protein comprising one or more heterologous protein domains A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### VII. METHODS OF USE

In some embodiments, the present disclosure provides methods for immunotherapy comprising administering an effective amount of the immune cells of the present disclosure. In one embodiments, a medical disease or disorder is treated by transfer of an immune cell population that elicits an immune response. In certain embodiments of the present disclosure, cancer or infection is treated by transfer of an immune cell population that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and viral infections.

Tumors for which the present treatment methods are useful include any malignant cell type, such as those found in a solid tumor or a hematological tumor. In some embodiments, the cancer is a CD22-positive cancer. In some embodiments, the cancer has a low expression of CD22 (e.g. a CD22 low cancer) In some embodiments, the cancer is a CD19-positive cancer. In some embodiments, the cancer has a low expression of CD19 (e g, a CD19 low cancer).

Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include but are not limited to tumors of the bone marrow, T or B cell malignancies, myeloid malignancies, leukemias, lymphomas, blastomas, myelomas. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant: cholangiocarcinoma; hepatocellular carcinoma, combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma, papillary and follicular adenocarcinoma; nonencapsulating sclerosing carcinoma; adrenal cortical carcinoma; endometroid carcinoma; skin appendage carcinoma, apocrine adenocarcinoma; sebaceous adenocarcinoma; ceruminous adenocarcinoma, mucoepidermoid carcinoma; cystadenocarcinoma; papillary cystadenocarcinoma; papillary serous cystadenocarcinoma; mucinous cystadenocarcinoma, mucinous adenocarcinoma; signet ring cell carcinoma; infiltrating duct carcinoma; medullary carcinoma; lobular carcinoma; inflammatory carcinoma; paget's disease, mammary; acinar cell carcinoma, adenosquamous carcinoma; adenocarcinoma w/squamous metaplasia; thymoma, malignant; ovarian stromal tumor, malignant, thecoma, malignant; granulosa cell tumor, malignant; androblastoma, malignant; Sertoli cell carcinoma, leydig cell tumor, malignant; lipid cell tumor, malignant; paraganglioma, malignant; extra-mammary paraganglioma, malignant, pheochromocytoma; glomangiosarcoma, malignant melanoma; amelanotic melanoma, superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma, fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant, mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma, glioma, malignant: ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; T lymphoblastic leukemia; T lymphoblastic lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL, high grade lymphoblastic NHL; high grade small non-cleaved cell NHL, bulky disease NHL; mantle cell lymphoma, AIDS-related lymphoma; Waldenstrom's macroglobulinemia; malignant histiocytosis; multiple myeloma; mast cell sarcoma, immunoproliferative small intestinal disease; leukemia, lymphoid leukemia; plasma cell leukemia; erythroleukemia; lymphosarcoma cell leukemia, myeloid leukemia, basophilic leukemia; eosinophilic leukemia; monocytic leukemia; mast cell leukemia, megakaryoblastic leukemia; myeloid sarcoma; hairy cell leukemia; chronic lymphocytic leukemia (CLL); chronic myeloid leukemia, acute lymphoblastic leukemia (ALL); acute lymphoblastic lymphoma, acute myeloid leukemia (AML); myelodysplastic syndrome (MDS); myeloproliferative neoplasms; chronic myeloblasts leukemia; diffuse large B-cell lymphoma (DLBCL); peripheral T-cell lymphoma (PTCL); or anaplastic large cell lymphoma (ALCL).

Particular embodiments concern methods of treatment of leukemia. Leukemia is a cancer of the blood or bone marrow and is characterized by an abnormal proliferation (production by multiplication) of blood cells, usually immature white blood cells (leukocytes). It is part of the broad group of diseases called hematological neoplasms. Leukemia is a broad term covering a spectrum of diseases. Leukemia is clinically and pathologically split into its acute and chronic forms and/or by and the cell type of origin (myeloid or lymphoid). In some embodiments, the leukemia is an antigen-low leukemia. In some embodiments, the leukemia is a CD22-low leukemia.

In certain embodiments of the present disclosure, immune cells are delivered to an individual in need thereof, such as an individual that has cancer or an infection. The cells then enhance the individual's immune system to attack or directly attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, 8, 9, 10, 11, or 12 or more weeks.

Certain embodiments of the present disclosure provide methods for treating or preventing an immune-mediated disorder. In one embodiment, the subject has an autoimmune disease. Non-limiting examples of autoimmune diseases include alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Bechet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erythematosus. Meniere's disease, mixed connective tissue disease, multiple sclerosis, type 1 or immune-mediated diabetes mellitus, myasthenia gravis, nephrotic syndrome (such as minimal change disease, focal glomerulosclerosis, or membranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosus, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease GVHD is a possible complication of any transplant that uses or contains stem cells from either a related or an unrelated donor. There are two kinds of GVHD, acute and chronic. Acute GVHD appears within the first three months following transplantation Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild, stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In some embodiments, the subject can be administered nonmyeloablative lymphodepleting immunotherapy prior to the immune cell therapy. The nonmyeloablative lymphodepleting immunotherapy can be any suitable such therapy, which can be administered by any suitable route. The nonmyeloablative lymphodepleting immunotherapy can comprise, for example, the administration of an anti-CD52 agent or anti-CD20 agent. In some embodiments, the lymphodepleting immunotherapy is an anti-CD52 antibody. In some embodiments, the anti-CD52 antibody is alemtuzumab. In some embodiments, the lymphodepleting immunotherapy is an anti-CD20 antibody Exemplary anti-CD20 antibodies include, but are not limited to rituximab, ofatumumab, ocrelizumab, obinutuzumab, ibritumomab or iodine i131 tositumomab. An exemplary route of administering anti-CD52 agent or anti-CD20 agent is intravenously. Likewise, any suitable dose of anti-CD52 agent or anti-agent can be administered.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The immune cell population can be administered in treatment regimens consistent with the disease, for example a single or a few doses over one to several weeks to ameliorate a disease state or periodic doses over an extended time to inhibit disease progression and prevent disease recurrence. The precise dose to be employed in the formulation will also depend on the route of administration, and the seriousness of the disease or disorder. The therapeutically effective amount of immune cells will be dependent on the subject being treated, the severity and type of the affliction, and the manner of administration. In some embodiments, doses that could be used in the treatment of human subjects range from at least 3.8×10⁴, at least 3 8×10⁵, at least 3.8×10⁶, at least 3.8×10⁷, at least 3.8×10⁹, at least 3.8×10⁹, or at least 3.8×10¹⁰ immune cells/m². In a certain embodiment, the dose used in the treatment of human subjects ranges from about 3.8×10⁹ to about 3.8×10⁹ immune cells/m². In additional embodiments, a therapeutically effective amount of immune cells can vary from about 5×10⁶ cells per kg body weight to about 7.5×10⁸ cells per kg body weight, such as from about 2×10⁷ cells to about 5×10⁸ cells per kg body weight, or from about 5×10⁷ cells to about 2×10⁸ cells per kg body weight, or from about 5×10⁶ cells per kg body weight to about 1×10⁷ cells per kg body weight. In some embodiments, a therapeutically effective amount of immune cells ranges from about 1×10⁵ cells per kg body weight to about 10×10⁹ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetyls alicylic acid, ibuprofen or naproxen sodium), cytokine antagonists (for example, anti-TNF and anti-IL-6), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

### 1. Pharmaceutical Compositions

Also provided herein are pharmaceutical compositions and formulations comprising immune cells (e.g., T cells) and a pharmaceutically acceptable carrier.

In some embodiments, a pharmaceutical composition comprises a dose ranging from about 1×10⁵ T cells to about 1×10⁹ T cells. In some embodiments, the dose is about 1×10⁵, 1×10⁶, 1×10⁷, 1×10⁸ or 1×10⁹ T cells. In some embodiments, a pharmaceutical composition comprises a dose ranging from about 5×10⁵ T cells to about 10×10¹² T cells.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA, sugars such as sucrose, mannitol, trehalose or sorbitol; salt-forming counter-ions such as sodium, metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### 2. Combination Therapies

In some embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RN A therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1), eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate, an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, decitabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone, teniposide; edatrexate; daunomycin; aminopterin; xeloda; ibandronate; irinotecan (e.g., CPT-11); topoisomerase inhibitor RFS 2000: difluoromethylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above. In some embodiments, azacitidine is administered at 75 mgs/m² subcutaneously.

### B. Radiotherapy

Other factors that cause DNA damage and have been used extensively include what are commonly known as γ-rays. X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### C. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target Various effector cells include cytotoxic T cells and NK cells.

Antibody-drug conjugates have emerged as a breakthrough approach to the development of cancer therapeutics Cancer is one of the leading causes of deaths in the world Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al, 1998); cytokine therapy, e.g., interferons α, β, and γ, IL-1, GM-CSF, and TNF (Bukowski et al. 1998, Davidson et at, 1998; Hellstrand et al, 1998); gene therapy, e.g., TNF, IL-1, IL-2, and p53 (Qin et al, 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al, 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA) In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718, Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2 In another embodiment, a PDL1 binding antagonist is a molecule that inhibits the binding of PDL1 to its binding partners. In a specific aspect, PDL1 binding partners are PD-1 and/or B7-1 In another embodiment, the PDL2 binding antagonist is a molecule that inhibits the binding of PDL2 to its binding partners. In a specific aspect, a PDL2 binding partner is PD-1 The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference Other PD-1 axis antagonists for use in the methods provided herein are known in the art such as described in U S. Patent Application No US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-0l 1.

In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224 Nivolumab, also known as MDX-1106-04. MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD 152. The complete cDNA sequence of human CTLA-4 has the Genbank accession number L15006. CTLA-4 is found on the surface of T cells and acts as an “off switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17). 10067-10071; Camacho et al. (2004)/Clin Oncology 22(145) Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos WO1995001994 and WO1998042752; all incorporated herein by reference, and immunoadhesins such as described in U.S. Pat. No. 8,329,867, incorporated herein by reference.

### 3. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 4. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VIII. DOSAGE REGIMENS

In one embodiment, the immune effector cells (e.g., T cells) are modified by engineering/introducing chimeric antigen receptors (e.g., anti-CD22 and anti-CD19 CAR) into said immune effector cells and then infused into a subject. In some embodiments, immune effector cells are modified by engineering/introducing a chimeric receptor, and functional effector element and/or a cytokine into the immune effector cells and then infused within about 0 days, within about 1 day, within about 2 days, within about 3 days, within about 4 days, within about 5 days, within about 6 days or within about 7 days into a subject.

In some embodiments, an amount of modified effector cells is administered to a subject in need thereof and the amount is determined based on the efficacy and the potential of inducing a cytokine-associated toxicity. In another embodiment, the modified effector cells are CAR⁺ and CD56⁺ cells. In some embodiments, an amount of modified effector cells comprises about 104 to about 10⁹ modified effector cells/kg. In some cases, an amount of modified effector cells comprises about 10⁴ to about 10⁵ modified effector cells/kg. In some cases, an amount of modified effector cells comprises about 10⁵ to about 10⁶ modified effector cells/kg. In some cases, an amount of modified effector cells comprises about 10⁶ to about 10⁷ modified effector cells/kg. In some cases, an amount of modified effector cells comprises about 10′ to about 10⁸ modified effector cells/kg. In some cases, an amount of modified effector cells comprises about 10⁸ to about 10⁹ modified effector cells/kg. In some cases, am amount of modified effector cells comprises about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, about 1×10⁹ modified effector cells/kg.

In one embodiment, the modified immune effector cells are targeted to the cancer via regional delivery directly to the tumor tissue. For example, in ovarian or renal cancer, the modified immune effector cells can be delivered intraperitoneally (IP) to the abdomen or peritoneal cavity. Such IP delivery can be performed via a port or pre-existing port placed for delivery of chemotherapy drugs. Other methods of regional delivery of modified immune effector cells can include catheter infusion into resection cavity, ultrasound guided intratumoral injection, hepatic artery infusion or intrapleural delivery.

In one embodiment, a subject in need thereof, can begin therapy with a first dose of modified immune effector cells delivered via IV followed by a second dose of modified immune effector cells delivered via IV. In one embodiment, a subject in need thereof, can begin therapy with a first dose of modified immune effector cells delivered via IP followed by a second dose of modified immune effector cells delivered via IV. In a further embodiment, the second dose of modified immune effector cells can be followed by subsequent doses which can be delivered via IV or IP. In one embodiment, the duration between the first and second or further subsequent dose can be about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days. In one embodiment, the duration between the first and second or further subsequent dose can be about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In some embodiments, the duration between the first and second or further subsequent dose can be about: 0, 1, 2, 3, 4, 5, 6, 7, 8, 9 or 10 years.

In another embodiment, a catheter can be placed at the tumor or metastasis site for further administration of 1, 2, 3, 4, 5, 6, 7, 8, 9, 10 doses of modified immune effector cells. In some cases, doses of modified effector cells can comprise about 10² to about 10⁹ modified effector cells/kg. In cases where toxicity is observed, doses of modified effector cells can comprise about 10² to about 10⁵ modified effector cells/kg. In some cases, doses of modified effector cells can start at about 10² modified effector cells/kg and subsequent doses can be increased to about: 10⁴, 10⁵, 10⁶, 10⁷, 10⁸ or 10⁹ modified effector cells/kg.

### IX. ARTICLES OF MANUFACTURE OR KITS

An article of manufacture or a kit is provided comprising immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or poly olefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### X. REFERENCES

Maude, S. L. et al. Chimeric Antigen Receptor T Cells for Sustained Remissions in Leukemia. N. Engl. J. Med. 371, 1507-1517 (2014).
Lee, D. W. et al. T cells expressing CD19 chimeric antigen receptors for acute lymphoblastic leukaemiain children and young adults: a phase 1 dose-escalation trial. Lancet 385, 517-528 (2015).
Park, J. H. et al. Long-Term Follow-up of CD19 CAR Therapy in Acute Lymphoblastic Leukemia. N. Engl. J. Med. 378, 449-459 (2018).
Gardner, R. et al. Acquisition of a CD19-negative myeloid phenotype allows immune escape of MLL-rearranged B-ALL from CD19 CAR-T-cell therapy. Blood 127, 2406-2410 (2016).
Turtle, C. J. et al. CD19 CAR-T cells of defined CD4+:CD8+ composition in adult B cell ALL patients. J. Clin. Invest. 126, 2123-2138 (2016).
Gardner, R A. et al. Intent to treat leukemia remission by CD19 CAR T cells of defined formulation and dose in children and young adults. Blood blood-2017-02-769208 (2017). doi:10.1182/blood-2017-02-769208
Maude, S. L. et al. Tisagenlecleucel in Children and Young Adults with B-Cell Lymphoblastic Leukemia. N. Engl. J. Med. 378, 439-448 (2018).
Pasquini, M. C. et al. Real-world evidence of tisagenlecleucel for pediatric acute lymphoblastic leukemia and non-Hodgkin lymphoma. Blood Adv. 4, (2020).
Wudhikarn, K. et al. Interventions and Outcomes of Adult Patients with B-ALL Progressing After CD19Chimeric Antigen Receptor T Cell Therapy. Blood (2021). doi:10.1182/blood.2020009515
Myers, R. M. et al. Humanized CD19-Targeted Chimeric Antigen Receptor (CAR) T Cells in CAR-Naive and CAR-Exposed Children and Young Adults With Relapsed or Refractory Acute Lymphoblastic Leukemia. J. Clin. Oncol. JCO.20.03458 (2021). doi:10.1200/JCO.20.03458
Fry, T. J. et al. CD22-targeted CAR T cells induce remission in B-ALL that is naive or resistant to CD19-targeted CAR immunotherapy. Nat. Med. 24, 20-28 (2018).
Shah, N. N. et al. CD4/CD8 T-Cell Selection Affects Chimeric Antigen Receptor (CAR) T-Cell Potency and Toxicity: Updated Results From a Phase I Anti-CD22 CAR T-Cell Trial. J. Clin. Oncol. 38, 1938-1950 (2020).
Ramakrishna, S. et al. Modulation of Target Antigen Density Improves CAR T-cell Functionality and Persistence. Clin. Cancer Res. (2019). doi:10.1158/1078-0432.CCR-18-3784
Kohler, M. E., Walsh, Z., Degolier, K. & Fry, T. J. Enhancing the Antigen-Sensitivity of the CD22 CAR through Modulation of the Affinity and Linker-Length of the Single-Chain Fragment Variable. Blood 136, 41-42 (2020).
Walsh, Z., Yang, Y. & Kohler, M. E. Immunobiology of chimeric antigen receptor T cells and novel

### XI. EXAMPLES

### Example 1—Bicistronic CD3ε-CARs Increases Antigen Sensitivity and Efficacy in Vivo

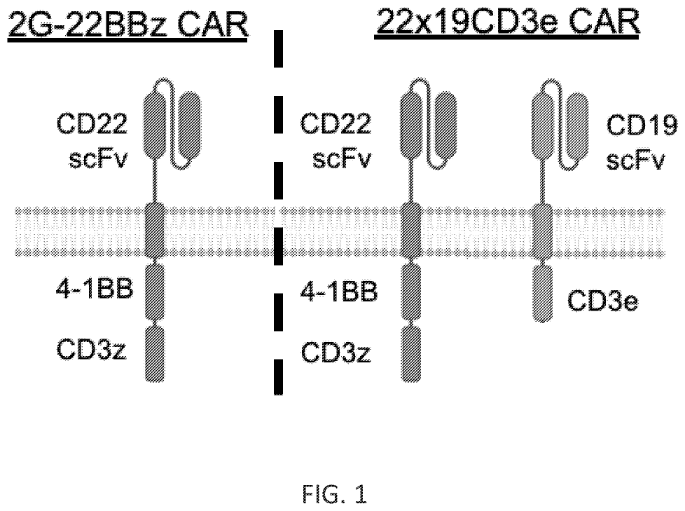

The bicistronic “22×19CD3e” CAR (also referred to as bicistronic “CD3ε-CAR”) construct was generated by cloning a 2^{nd} generation CAR targeting CD22 (SEQ ID NO: 86) with the m971 scFv, a CD8a TM domain, a 4-1BB costimulatory domain and the signaling domain of CD3-zeta into a bicistronic construct with a CAR targeting CD19 (SEQ ID NO: 59) with the FMC63 scFv, a CD28 transmembrane domain and a CD3ε intracellular domain (FIG. 1, right panel). This was cloned into a 3^{rd} Generation Lentivirus transfer plasmid and replication-incompetent Lentivirus was generated by established lentivirus protocols. T cells from a healthy donor were activated and transduced with lentivirus encoding CD3ε-CART construct using standard transduction techniques. In parallel, T cells from the same healthy donor were transduced with a standard 2^{nd} generation CD22 CAR with a 4-1BB costimulatory domain (“2G-22BBz” which is identical to the first CAR of the CD3ε-CART format) using the same transduction techniques. FIG. 1 shows a schematic depicting a bicistronic 22×19CD3ε CAR (SEQ ID NO: 88, which has a first CAR of SEQ ID NO: 86 and a second CAR of SEQ ID NO: 59) and a standard 2G-22BBz CAR construct (SEQ ID NO: 86).

NALM6 is a B cell acute lymphoblastic leukemia cell line which naturally expresses high levels of the CD22 antigen (“CD22-High NALM6” cells). This cell line has been engineered to express low levels of the CD22 antigen (“CD22-Low NALM6” cells) which are resistant to the standard 2^{nd} generation CD22 CAR (2G-22BBz) which has been shown to have efficacy in clinical trials.

NSG mice were inoculated with 10⁶ CD22-Low NALM6 cells and treated with either 3×10⁶ 2G-22BBz CAR T cells or 3×10⁶ 22×19CD3ε CAR T cells and leukemia progression/regression was monitored by bioluminescent imaging (BLI) twice weekly for 42 days, and compared to mice that were inoculated with 10⁶ CD22-Low NALM6 cells and received no CAR T cells (No Tx).

Mice were engrafted with 10⁶ of either CD22-Low NALM6 or CD22-High NALM6 cells on Day −3. All leukemia cells express the luciferase enzyme, which allows for in vivo bioluminescent imaging (BLI) to monitor the total amount of leukemia in the mouse. On Day 0, mice were injected with 3×10⁶ 2G-22BBz CAR T cells or 22×19CD3ε CAR (comprising SEQ ID NO: 88) T cells. A group of mice received no treatment (No Tx) to show the natural progression of untreated leukemia. Mice were monitored by BLI twice weekly to monitor leukemia burden.

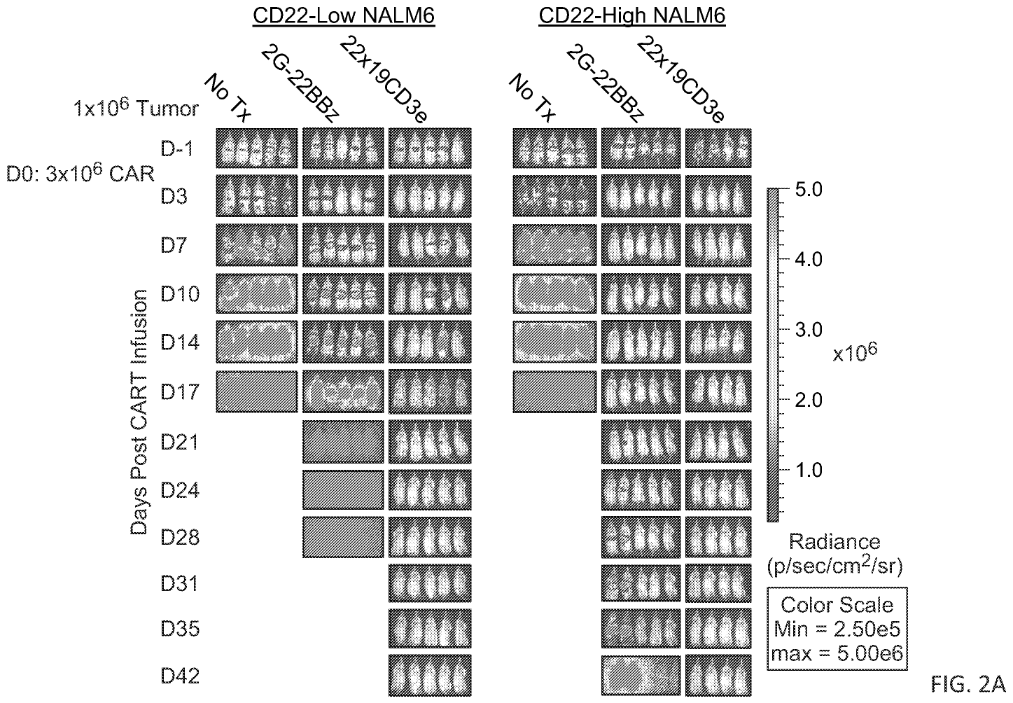

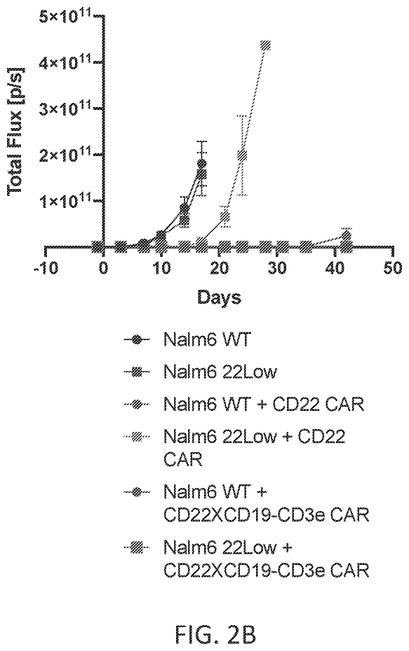

As shown in FIG. 2A, mice treated with 22×19CD3ε CAR T cells were able to efficiently eradicate leukemia from mice regardless of the amount of CD22 antigen present on the leukemia. Conversely, mice treated with the standard 2G-22BBz CAR T cells showed minimal slowing of CD22-Low leukemia progression and an initial clearance of CD22-High leukemia followed by relapse of the CD22-High leukemia. As shown in FIG. 2A (left, “CD22-Low NALM6”), only mice treated with 22×19CD3ε CAR T cells were able to clear the CD22-Low NALM6 leukemia cells, whereas mice treated with 2G-22BBz CAR T cells had leukemia progression due to the level of CD22 being too low to be effectively targeted by this CAR. All five mice receiving bicistronic 22×19CD3ε CAR-T cell treatment survived 42 days following treatment with CAR T cells, and leukemia cells were undetectable by BLI by day 24. These results demonstrate that bicistronic 22×19CD3ε CAR T cells are effective against CD22-Low leukemia that is otherwise untreatable with standard 2^{nd} generation CAR T cell therapies.

NSG Mice were also inoculated with 10⁶ CD22-High NALM6 cells and treated with either 3×10⁶ 2G-22BBz CAR T cells or 3×10⁶ 22×19CD3ε CAR T cells and leukemia progression/regression was monitored by BLI twice weekly for out to 42 days. As shown in FIG. 2A (right; “CD22-High NALM6”), the 2G-22BBz CAR T cells are initially effective in clearing the leukemia, but ultimately, a subset of mice relapse when receiving 2G-22BBz CAR T cells. In contrast, mice treated with the same number of 22×19CD3e CAR T cells cleared CD22-High leukemia without relapse or recurrence of disease 42 days after receiving CAR T cells. These data demonstrate that the 22×19CD3ε CAR of the present disclosure is more efficacious in vivo in settings of low- and high-levels of antigen.

These data demonstrate the enhanced ability of 22×19CD3e CAR T cells to eradicate leukemia that is not completely eradicated by standard 2G-22BBz CAR T cells.

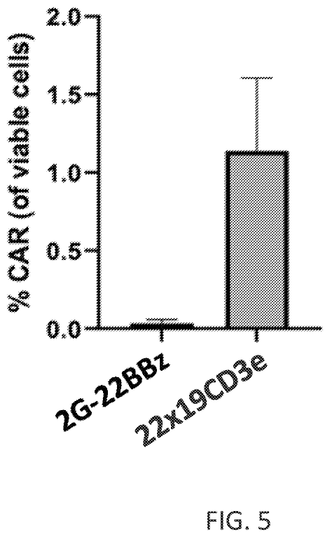

On day 50 following treatment in mice receiving CD22-High NALM6 cells, bone marrow samples from the two cohorts of mice were analyzed by flow cytometry to determine the percentage of CAR T cells present in the marrow. The percentage of viable cells that were CAR T cells was analyzed. As shown in FIG. 5, mice receiving 22×19CD3ε (“CD3e”) CAR T cells showed increased amounts of CAR T cells as a percentage of viable cells (about 1.2%) in the bone marrow compared to mice receiving 2G-22BBz (“2G”) CAR T cells.

Without wishing to be bound by theory, these results indicate that the bicistronic 22×19CD3e CAR constructs of the present disclosure are more effective at treating leukemia, including leukemia characterized by low-antigen expression, as compared to standard 2^{nd} generation CAR constructs, and that the bicistronic 22×19CD3ε CAR constructs exhibit increased persistence as compared to existing 2^{nd} generation CAR constructs.

### Example 2—22×19CD3ε-CAR T Cells have Higher Cytotoxicity Against CD22-Low and CD22-High NALM6 Leukemia

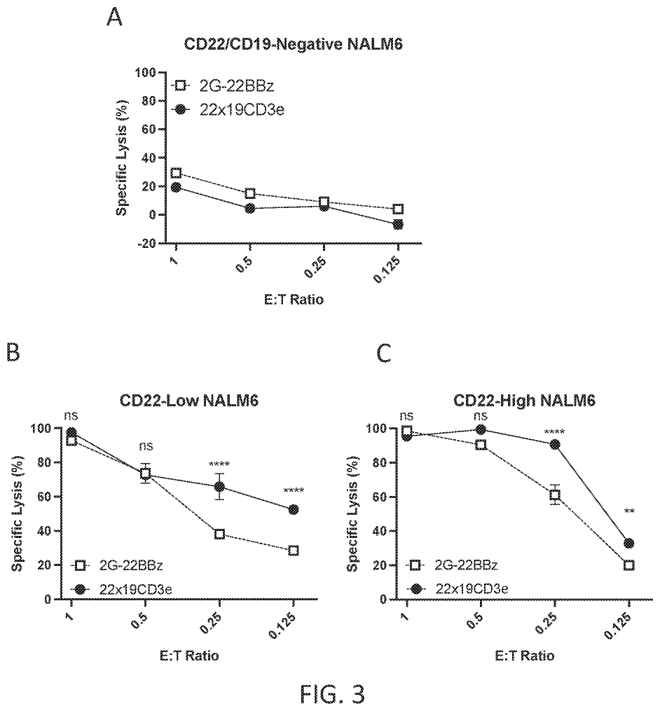

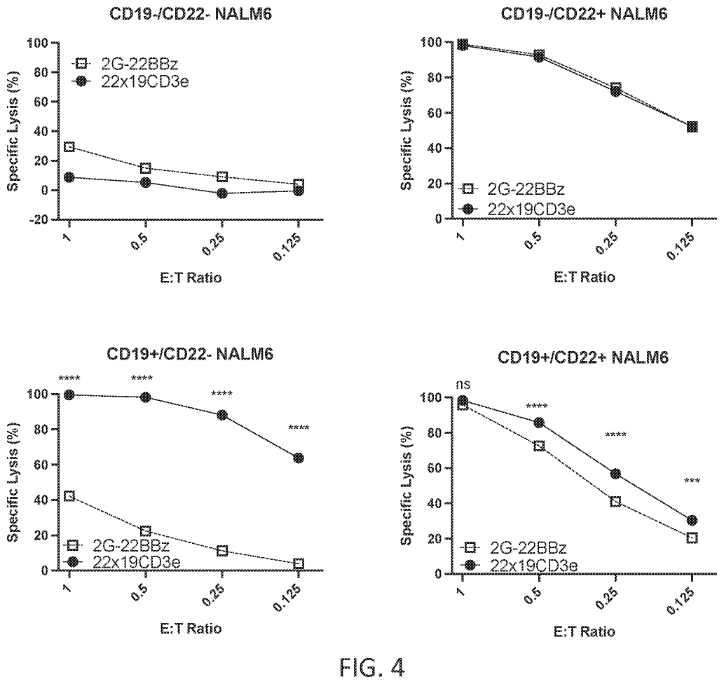

T cells from the same healthy donor were transduced in parallel with the standard 2nd generation 2G-22BBz construct or the bicistronic 22×19CD3e construct of the present disclosure according to standard laboratory protocols. The resultant CAR T cells were co-cultured with NALM6 cells that either expressed no antigen (CD22/CD19-Negative), low levels of antigen (CD22-Low) or wild type levels of antigen (CD22-High) for 16 hour at various ratios (1, 0.5, 0.25, and 0.125) of CAR T cells (Effector) to NALM6 cells (Targets) (E:T ratio) and killing of the NALM6 cells (specific lysis) was measured at the end of the co-culture (FIGS. 3A-C). Luciferase activity of surviving NALM6 cells was used to calculate the killing efficiency (Specific Lysis) of each type of CAR T cell.

As shown in FIGS. 3B and 3C, bicistronic 22×19CD3ε CAR T cells (black circles) were more effective at killing CD22-Low (E:T ratio of 0.25 and 0.125) and CD22-High (ET ratio of 0.5, 0.25 and 0.125) NALM6 cells at low E:T ratios when compared to standard 2G-22BBz CAR T cells (white squares). CD22/CD19-Negative NALM6, which acts as a measure of non-specific killing by the CAR T cells, was not different between 2G-22BBz (white squares) or 22×19CD3e (black circles) CAR T cells, indicating the expression of 22×19CD3e does not increase indiscriminate toxicity of the CAR T cells. Upon co-culture with CD22-Low NALM6 or CD22-High NALM6, the 22×19CD3e CAR T cells showed statistically significant increased killing of NALM6 cells relative to 2G-22BBz CAR T cells at lower E:T ratios. “ns” refers to statistically “non-significant” changes between the tested CAR T cell groups. “**” refers to a statically significant difference between 2G-22BBz and 22×19CD3e CAR T cell killing with a p-value of <0.01. “****” refers to a statically significant difference between 2G-22BBz and 22×19CD3e CAR T cell killing with a p-value of <0.0001.

### Example 3—22×19CD3E CAR T Cells Exhibit Cytotoxicity Against Leukemia Through Targeting Either the CD22 or the CD19 Antigens

T cells from a healthy donor were transduced in parallel with either the standard second generation 2G-22BBz CAR or the bicistronic 22×19CD3ε CAR according to standard laboratory protocols. The resultant CAR T cells were co-cultured with NALM6 cells engineered to expressed no antigen (CD19−/CD22− NALM6), CD22 only (CD19−/CD22+NALM6), CD19 only (CD19+/CD22− NALM6), or both antigens (CD19+/CD22+NALM6). CAR T cells (Effector; E) to NALM6 cells (Targets;T) (E:T ratio) co-cultured at various E:T ratios (1, 0.5, 0.25, and 0.125) and killing of NALM6 cells (specific lysis %) was measured after 16 hours (FIG. 4). Luciferase activity of surviving NALM6 cells was used to calculate the killing efficiency (Specific Lysis) of each type of CAR T cell.

As shown in FIG. 4, bicistronic 22×19CD3E CAR T cells (black circles) were able to kill NALM6 cells that expressed either CD22 (top, right) or CD19 (bottom, left) alone, whereas 2G-22BBz CAR T cells could only kill CD22+NALM6. CD19−/CD22− NALM6, which acts as a measure of non-specific killing by the CAR T cells was not significantly different between 2G-22BBz (white squares) or 22×19CD3e (black circles) CAR T cells, indicating the bicistronic 22×19CD3ε CAR expression does not increase indiscriminate toxicity of the CAR T cells. CD19−/CD22+NALM6 cells were killed equally well by 2G-22BBz and 22×19CD3e CAR T cells at all E:T ratios, indicating no antigen-independent activation of the second CAR of the 22×19CD3e CAR construct could be observed. CD19+/CD22−NALM6 cells were efficiently targeted by the 22×19CD3e CAR T cells of the present disclosure but not targeted by standard 2G-22BBz CAR T cells, indicating the second CAR of the 22×19CD3e construct is sufficient to elicit cytotoxicity. CD19+/CD22+NALM6 cells were killed by both tested CAR T cells, but the 22×19CD3e CAR T cells of the present disclosure demonstrated a statistically significant enhancement of leukemia cell killing when compared to the standard 2G-22BBz CAR T cells at lower E:T ratios. “ns” refers to statistically “non-significant” changes between the tested CAR T cell groups. “***” refers to a statically significant difference between 2G-22BBz and 22×19CD3e CAR T cell killing with a p-value of <0.001. “****” refers to a statically significant difference between 2G-22BBz and 22×19CD3e CAR T cell killing with a p-value of <0.0001. Taken together, this data demonstrates that the 22×19CD3e CAR can function as an “OR-CAR” with the ability to independently target two separate antigens, which broadens the specificity of the CAR-T therapeutic. Furthermore, these experiments again demonstrate enhanced killing by the 22×19CD3ε CAR T cells relative to 2G-22BBz CAR T cells when targeting both antigens simultaneously.

### Example 4—Increased Persistence of Bicistronic 22×19CD3ε CAR-T Cells. In Vivo

NSG Mice were inoculated with 10⁶ CD22-High NALM6 cells on Day −3 and treated with either 3×10⁶ 2G-22BBz CAR T cells or 3×10⁶ 22×19CD3ε CAR T cells on Day 0. On day 50 following treatment, bone marrow samples from the two cohorts of mice were analyzed by flow cytometry to determine the percentage of CAR T cells present in the marrow.

Mice were engrafted with 10⁶ CD22-High NALM6 cells on Day −3. All leukemia cells express the luciferase enzyme, which allows for in vivo bioluminescent imaging (BLI) to monitor the total amount of leukemia in the mouse. On Day 0, mice were injected with 3×10⁶ 2G-22BBz CAR T cells or 22×19CD3e CAR T cells. On Day 49 all mice were euthanized, and the bone marrow was collected and analyzed by flow cytometry for persistent human CAR T cells.

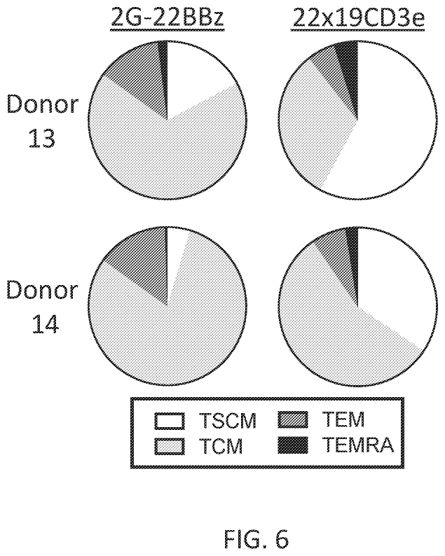

As shown in FIG. 5, the bicistronic 22×19CD3ε CART cells persist better in vivo than do standard 2G-22BBz CAR T cells. About 1.2% of viable cells in the bone marrow samples were 22×19CD3ε CAR T cells. These data demonstrate the enhanced persistence of 22×19CD3e CAR T cells relative to standard 2G-22BBz CAR T cells. This is likely due to the 22×19CD3ε CAR T cell product containing a higher amount of T cells with a T stem cell memory (TSCM) phenotype than what is seen in 2G-22BBz CAR T cell products (FIG. 6).

T cells from two independent healthy donors were transduced with either the 2G-22BBz CAR construct or bicistronic 22×19CD3e CAR construct to generate CAR T cells in parallel for comparison of the final products. Upon completion of CAR T cell production and expansion, CAR T cell products were analyzed by flow cytometry for expression of CAR, CD4, CD8, CD62L, CD45RA and CD95 to determine the proportion of CAR T cells with a T stem cell memory (TSCM), T central memory (TCM), T effector memory (TEM) or T effector memory with re-expression of CD45RA (TEMRA) phenotype (FIG. 6). Bicistronic 22×19CD3e CAR T cells were found to contain more CD4+ T cells with a TSCM phenotype than CD4+2G-22BBz CAR T cells. As T cells with the TSCM phenotype have been shown to have greater potential for persistence and improved in vivo efficacy upon adoptive transfer, this data explains the enhanced in vivo persistence of 22×19CD3e CAR T cells (shown in FIG. 5) and the increased efficacy of 22×19CD3e CAR T cells (shown in FIG. 2).

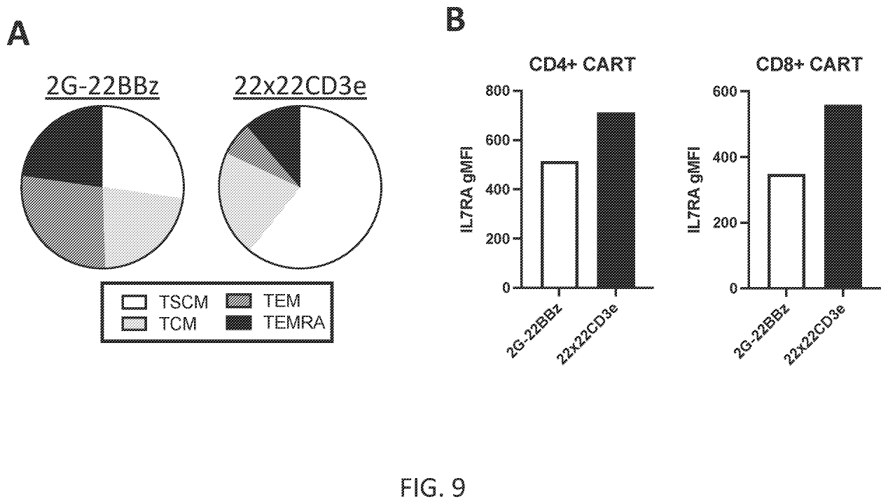

The promotion of this TSCM phenotype in the CAR product may be due to the incorporation of the CD3ε domain into the second CAR of the disclosed bicistronic construct, as the TSCM phenotype remains predominant in bicistronic CD3e CAR T cells, when the antigen specificity of the disclosed bicistronic CAR is changed from CD22 and CD19 to CD22 and CD22 (FIG. 9A).

### Example 5—Bicistronic CD3e-CAR Construct Increases Antigen Sensitivity when Targeting a Single Antigen

Results from prior experiments demonstrate that 22×19CD3ε CAR T cells are more effective at killing and eradicating CD22-Low leukemia in vitro and in vivo, as shown in FIG. 3 and FIG. 2, respectively.

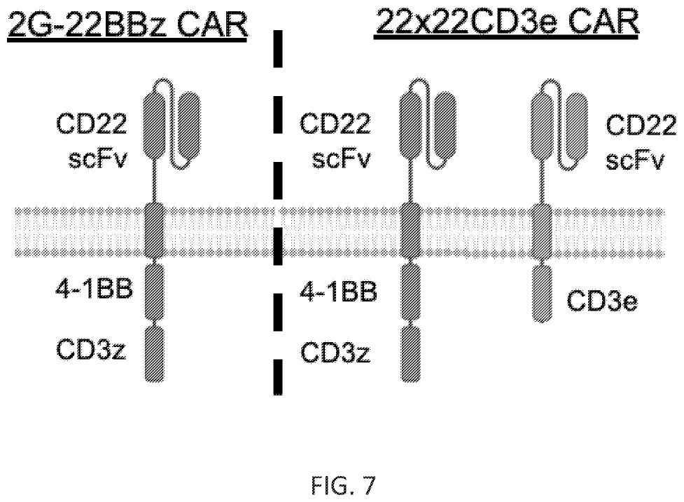

A second bicistronic construct (“22×22CD3ε CAR”) was constructed (SEQ ID NO: 95), in which the first CAR of the bicistronic construct targets the CD22 antigen (SEQ ID NO: 98) and comprises a CD22-binding scFv, a CD8 extracellular stalk and transmembrane domain, a 4-1BB costimulatory domain and a CD3z signaling domain (FIG. 7). This first CAR of the bicistronic construct (SEQ ID NO: 98) was paired with the second CAR of the bicistronic construct (SEQ ID NO: 90), which also targets CD22 through the same scFv and contains an extracellular stalk and transmembrane domain from the CD28 molecule and the signaling domain of the CD3ε protein. FIG. 7 shows a schematic of an exemplary bicistronic 22×22CD3ε CAR construct (bicistronic CAR of SEQ ID NO: 95, having a first CAR of SEQ ID NO: 98 and a second CAR of SEQ ID NO: 90; or bicistronic CAR of SEQ ID NO: 93, having a first CAR of SEQ ID NO: 100 and a second CAR of SEQ ID NO: 103) compared to the standard 2G-22BBz CAR construct (SEQ ID NO: 86).

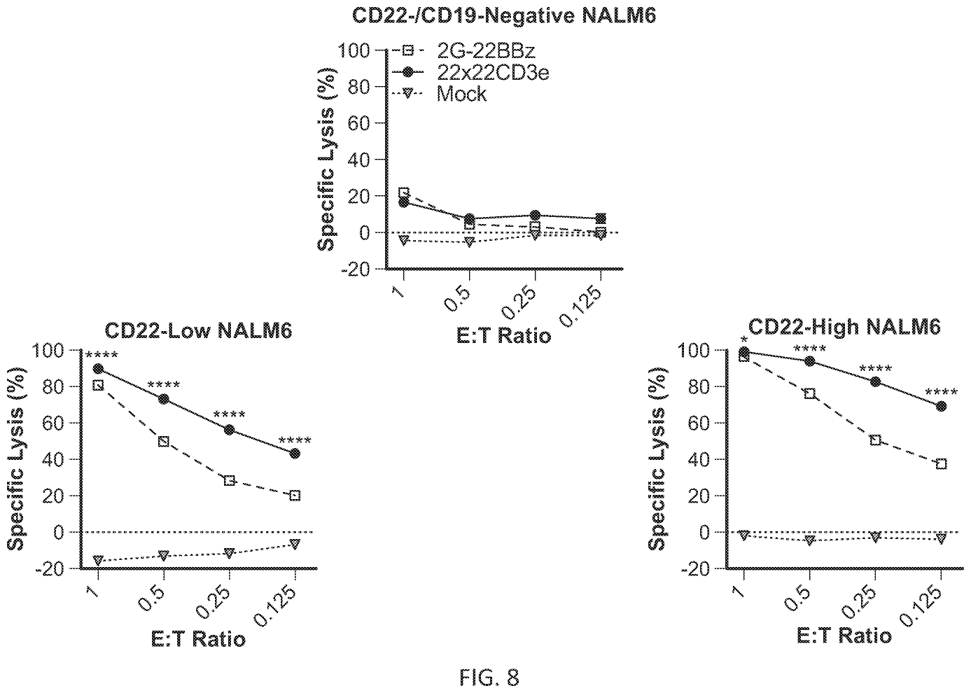

This 22×22CD3ε CAR is restricted solely to the recognition of the CD22 antigen and was transduced into T cells from a healthy donor according to standard protocols in parallel with the standard 2G-22BBz CAR. The resultant CAR T cells were tested for their ability to kill CD22-Low and CD22-High NALM6 cells at various E:T ratios during a 16 hour co-culture. 2G-22BBz and 22×22CD3e CAR T cells were co-cultured with NALM6 cells engineered to express no CD22 or CD19 antigen (CD22/CD19-Negative NALM6), low levels of CD22 antigen (CD22-Low NALM6) or wild type levels of CD22 antigen (CD22-High NALM6). CAR T cells (Effector; E) and NALM6 cells (Target; T) were co-incubated at multiple E:T ratios (1, 0.5, 0.25, and 0.125) and specific lysis of target NALM6 cells was assessed (FIG. 8).

Co-culture took place over 16 hour at various ratios of Effector (CAR T cells) to Targets (NALM6 cells) (E:T Ratio) and luciferase activity of surviving NALM6 cells was used to calculate the killing efficiency (Specific Lysis) of each type of CAR T cell. As shown in FIG. 8, the 22×22CD3ε CAR T cells (black circles) of the present disclosure demonstrated a statistically significant enhancement in the killing of NALM6 cells relative to 2G-22BBz CAR T cells (white squares) at an E:T ratio of 1, 0.5, 0.25 and 0.125. This occurred when the target antigen, CD22, was expressed at low levels (CD22-Low NALM6) indicating the ability of the 22×22CD3ε CAR to enhance the antigen-sensitivity of CAR T cells.

For CD22/CD19-Negative NALM6 cells, which acts as a measure of non-specific killing by the CAR T cells, there was no difference between 2G-22BBz (white squares) or 22×22CD3e (black circles) CAR T cells and no material difference from mock T cells (grey triangles), indicating the 22×22CD3e CAR expression does not increase indiscriminate toxicity of the CAR T cells when solely targeting the CD22 antigen. Upon co-culture with CD22-Low NALM6 or CD22-High NALM6, the 22×22CD3e CAR T cells showed statistically significant increased killing of NALM6 cells relative to 2G-22BBz CAR T cells at all E:T ratios. “*” refers to a statically significant difference between 2G-22BBz and 22×22CD3e CAR T cell killing with a p-value of <0.05. “****” refers to a statically significant difference between 2G-22BBz and 22×19CD3e CAR T cell killing with a p-value of <0.0001.

T cells from a healthy donor were transduced with either the 2G-22BBz CAR construct or the 22×22CD3e CAR construct of this present disclosure to generate CAR T cells in parallel for comparison of the final products. Upon completion of CAR T cell production and expansion, CAR T cell products were analyzed by flow cytometry for expression of CAR, CD4, CD8, CD62L, CD45RA, and CD95 to determine the proportion of CAR T cells with a T stem cell memory (TSCM), T central memory (TCM), T effector memory (TEM) or T effector memory with re-expression of CD45RA (TEMRA) phenotype (FIG. 9A). CAR T cells were also stained for expression of the IL-7 Receptor alpha-chain (IL7RA) (FIG. 9B).

FIG. 9A shows that 22×22CD3e CAR T cells contain more CD4+ T cells with a TSCM phenotype compared to CD4+ 2G-22BBz CAR T cells. FIG. 9B shows levels of IL7RA on both CD4+ and CD8+ 22×22CD3e CAR T cells compared to 2G-22BBz CAR T cells. As T cells with the TSCM phenotype have been shown to have greater potential for persistence and improved in vivo efficacy upon adoptive transfer and as IL7RA expression has been correlated with enhanced T cell persistence, this data suggests that the enhanced persistence and in vivo efficacy seen with the 22×19CD3e CAR T cells is inherent to the present disclosure of a CD3e-containing CAR in combination with a 2^{nd} generation CAR and is independent of which antigens are targeted with this bicistronic strategy.

What is claimed is:

1. A genetically modified immune cell comprising:
a) a first chimeric antigen receptor (CAR) comprising an antigen binding domain that binds to a first antigen, a transmembrane domain and an intracellular signaling domain;
b) a second CAR comprising an antigen binding domain that binds to a second antigen, a transmembrane domain and a CD3ε intracellular signaling domain.

2. The genetically modified immune cell of claim 1, wherein the first antigen and the second antigen are different.

3. The genetically modified immune cell of claim 1, wherein the first antigen and the second antigen are the same.

4. The genetically modified immune cell of any one of the preceding claims, wherein the intracellular signaling domain of the first CAR comprises a CD3zeta intracellular signaling domain.

5. The genetically modified immune cell of claim 4, wherein the CD3zeta intracellular signaling domain comprises the amino acid sequence of SEQ ID NO: 25 or SEQ ID NO: 26, preferably wherein the CD3zeta intracellular signaling domain comprises the amino acid sequence of SEQ ID NO: 25.

6. The genetically modified immune cell of any one of the preceding claims, wherein the intracellular signaling domain of the first CAR further comprises at least one additional intracellular signaling domains selected from the group consisting of a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8α intracellular signaling domain, an OX40 intracellular signaling domain, a 4-4BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain and any combination thereof.

7. The genetically modified immune cell of claim 6, wherein the at least one additional intracellular signaling domain is a 4-1BB intracellular signaling domain comprising the amino acid sequence of SEQ ID NO: 18.

8. The genetically modified immune cell of any one of the preceding claims, wherein the CD3ε intracellular signaling domain of the second CAR comprises the amino acid sequence of SEQ ID NO: 27 or SEQ ID NO: 28, preferably wherein the CD3ε intracellular signaling domain of the second CAR comprises the amino acid sequence of SEQ ID NO: 28.

9. The genetically modified immune cell of any one of the preceding claims, wherein the transmembrane domain of the first CAR and/or the second CAR is derived from a transmembrane domain selected from the group consisting of a CD8α transmembrane domain, a CD28 transmembrane domain, a CD3z transmembrane domain, a CD3ε transmembrane domain, a CD4 transmembrane domain, a 4-1BB transmembrane domain, a OX40 transmembrane domain, a ICOS transmembrane domain, a PD-1 transmembrane domain, a LAG-3 transmembrane domain, a 2B4 transmembrane domain, a BTLA transmembrane domain and any combination thereof.

10. The genetically modified immune cell of claim 9, wherein the transmembrane domain of the first CAR is derived from a CD8alpha transmembrane domain comprising the amino acid sequence of SEQ ID NO: 13.

11. The genetically modified immune cell of claim 9 or claim 10, wherein the transmembrane domain of the second CAR is derived from a CD3ε transmembrane domain comprising the amino acid sequence of SEQ ID NO: 15.

12. The genetically modified immune cell of any one of claims 1-11, wherein the antigen binding domain of the first CAR and/or the antigen binding domain of the second CAR is an antibody, an antibody fragment, a single chain antibody, a single domain antibody, an scFv, a VH or a VHH or antigen binding fragment thereof.

13. The genetically modified immune cell of any one of claims 1-12, wherein the antigen binding domain of the first CAR and/or the antigen binding domain of the second CAR further comprises a leader domain selected from the group consisting of a CD8α leader domain and a IgG signal peptide domain.

14. The genetically modified immune cell of claim 13, wherein the leader domain is a CD8α leader domain comprising the amino acid sequence of SEQ ID NO: 1 or SEQ ID NO: 2.

15. The genetically modified immune cell of claim 13, wherein the leader domain is a IgG signal peptide domain comprising the amino acid sequence of SEQ ID NO: 4.

16. The genetically modified immune cell of any one of claims 1-15, wherein the first antigen and the second antigen is a tumor associated antigen.

17. The genetically modified immune cell of claim 16, wherein the tumor associated antigen is selected from a group consisting of CD19, CD22, CD20, CD138, BCMA, CD33, CD123, FLT, CLL, CD56, CD34, CD117, CD14, CD133, CD44v6, CD47, CD64, CD96, CD97, CD99, CD45, CD9, Muc1, Lewis-Y, IL1RAP, FR-beta, CD5, CD7, CD38, CD30, B7-H3, HER2, CD44v6, CEA, c-Met, EGFRvIII, Epcam, EphA2, FR-alpha, GD2, GPC3, IL13R-alpha2, IL11R-alpha, L1-CAM, mesothelin, MUC1, MUC16, NKGD2 and PSCA.

18. The genetically modified immune cell of claim 17, wherein the first antigen is CD22.

19. The genetically modified immune cell of claim 17 or claim 18, wherein the second antigen is CD19.

20. The genetically modified immune cell of claim 17 or 18, wherein the second antigen is CD22.

21. The genetically modified immune cell of any one of claims 1-20, wherein the immune cell is a T-cell, a Natural Killer (NK) cell, a Natural Killer (NK)-like cell, a Cytokine Induced Killer (CIK) cell, a hematopoietic progenitor cell, a peripheral blood (PB) derived T cell or an umbilical cord blood (UCB) derived T-cell.

22. The genetically modified immune cell of claim 21, wherein the immune cell is a T-cell.

23. The genetically modified immune cell of any one of the preceding claims, wherein the first CAR comprises the amino acid sequence of SEQ ID NO: 57, 86, 98, 100.

24. The genetically modified immune cell of any one of the preceding claims, wherein the second CAR comprises the amino acid sequence of SEQ ID NO: 59, 90 or 103.

25. The genetically modified immune cell of claim 1, wherein the first CAR comprises the amino acid sequence of SEQ ID NO: 86 and the second CAR comprises SEQ ID NO: 59.

26. The genetically modified immune cell of claim 1, wherein the first CAR comprises the amino acid sequence of SEQ ID NO: 98 and the second CAR comprises SEQ ID NO: 90.

27. A composition comprising the genetically modified immune cell of any one of claims 1-26 and a pharmaceutically acceptable carrier.

28. A composition comprising a population of cells, wherein the plurality of cells of the population comprises the genetically modified immune cell of any one of claims 1-26.

29. The composition of claim 28, wherein the plurality of the cells of the population comprises at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90/c, 95%, 97%, 99% or any percentage in between of the genetically modified immune cell of any one of claims 1-18.

30. A polynucleotide encoding the first CAR and the second CAR of the genetically modified immune cell of any one of claims 1-26.

31. The polynucleotide of claim 30, wherein a nucleic acid sequence encoding a self-cleaving peptide sequence is located in between the nucleic acid sequence encoding the first CAR and the nucleic acid sequence encoding the second CAR.

32. The polynucleotide of claim 31, wherein the self-cleaving peptide sequence comprises the amino acid sequence of SEQ ID NO: 67.

33. The polynucleotide of any one of claims 30-32, wherein the first CAR and the second CAR encoded on a single vector.

34. The polynucleotide of claim 33, wherein the vector is a viral vector, a lentivirus vector, a non-viral vector or a transposon.

35. The polynucleotide of claim 34, wherein the vector is a bicistronic lentiviral vector.

36. A method of producing a population of genetically modified immune cells, comprising:
a) introducing into a plurality of immune cells a composition comprising the polynucleotide sequence of any one of claims 30-35, thereby generating a population of genetically modified immune cells;
b) culturing the population of genetically modified immune cells under conditions suitable for integration of the polynucleotide sequence;
c) expanding and/or selecting at least one cell from the population of genetically modified immune cells that expresses the first CAR and the second CAR on the cell surface.

37. A method of treating cancer in a subject in need thereof comprising administrating the composition of any one of claims 27-29.

38. The method of claim 37, wherein the administration of a composition comprising a modified immune cell comprising first CAR and the second CAR increases the immune response against a target cell in comparison to the administration of a composition comprising a modified immune cell comprising a first CAR alone.

39. The method of claim 38, wherein the increased immune response at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between greater than a composition comprising a modified immune cell comprising a first CAR alone.

40. The method of any one of claims 37-39, wherein the cancer is a solid tumor, a B cell malignancy, a myeloid malignancy, a T-cell malignancy, acute lymphoblastic leukemia, acute lymphoblastic lymphoma, Non-Hodgkin lymphoma, Hodgkins lymphoma, chronic lymphocytic leukemia, multiple myeloma, acute myeloid leukemia, myelodysplastic syndrome, myeloproliferative neoplasms, chronic myeloid leukemia, T lymphoblastic leukemia, T lymphoblastic lymphoma or Anaplastic Large Cell Leukemia.

41. The method of any one of claims 37-40, wherein the cancer has a low cell surface expression of the first antigen and/or a low cell surface expression of the second antigen.


SEQUENCE LISTING (104 sequences)

SEQ ID NO: 1 - protein, 25 aa, Homo sapiens
ASATMALPVTALLLPLALLLHAARP

SEQ ID NO: 2 - protein, 21 aa, Homo sapiens
MALPVTALLLPLALLLHAARP

SEQ ID NO: 3 - protein, 23 aa, Homo sapiens
GSMEFGLSWLFLVAILKGVQCSR

SEQ ID NO: 4 - protein, 21 aa, Homo sapiens
MEFGLSWLFLVAILKGVQCSR

SEQ ID NO: 5 - protein, 47 aa, Homo sapiens
LETTTPAPRPPTPAPTIASQPLSLRPEACRPAAGGAVHTRGLDFACD

SEQ ID NO: 6 - protein, 45 aa, Homo sapiens
TTTPAPRPPTPAPTIASQPLSLRPEACRPAAGGAVHTRGLDFACD

SEQ ID NO: 7 - protein, 41 aa, Homo sapiens
SRIEVMYPPPYLDNEKSNGTIIHVKGKHLCPSPLFPGPSKP

SEQ ID NO: 8 - protein, 39 aa, Homo sapiens
IEVMYPPPYLDNEKSNGTIIHVKGKHLCPSPLFPGPSKP

SEQ ID NO: 9 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 12 aa, Homo sapiens
EPKSCDKTHTCP

SEQ ID NO: 11 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 12 aa, Homo sapiens
ESKYGPPCPSCP

SEQ ID NO: 13 - protein, 24 aa, Homo sapiens
IYIWAPLAGTCGVLLLSLVITLYC

SEQ ID NO: 14 - protein, 27 aa, Homo sapiens
FWVLVVVGGVLACYSLLVTVAFIIFWV

SEQ ID NO: 15 - protein, 26 aa, Homo sapiens
VMSVATIVIVDICITGGLLLLVYYWS

SEQ ID NO: 16 - protein, 41 aa, Homo sapiens
RSKRSRGGHSDYMNMTPRRPGPTRKHYQPYAPPRDFAAYRS

SEQ ID NO: 17 - protein, 41 aa, Homo sapiens
RSKRSRLLHSDYMNMTPRRPGPTRKHYQPYAPPRDFAAYRS

SEQ ID NO: 18 - protein, 42 aa, Homo sapiens
KRGRKKLLYIFKQPFMRPVQTTQEEDGCSCRFPEEEEGGCEL

SEQ ID NO: 19 - protein, 24 aa, Homo sapiens
LCARPRRSPAQEDGKVYINMPGRG

SEQ ID NO: 20 - protein, 52 aa, Homo sapiens
YFLGRLVPRGRGAAEAATRKQRITETESPYQELQGQRSDVYSDLNTQRPYYK

SEQ ID NO: 21 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 22 - protein, 42 aa, Homo sapiens
ALYLLRRDQRLPPDAHKPPGGGSFRTPIQEEQADAHSTLAKI

SEQ ID NO: 23 - protein, 49 aa, Homo sapiens
HQRRKYRSNKGESPVEPAEPCHYSCPREEEGSTIPIQEDYRKPEPACSP

SEQ ID NO: 24 - protein, 48 aa, Homo sapiens
QRRKYRSNKGESPVEPAEPCHYSCPREEEGSTIPIQEDYRKPEPACSP

SEQ ID NO: 25 - protein, 114 aa, Homo sapiens
DIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 55 aa, Homo sapiens
KNRKAKAKPVTRGAGAGGRQRGQNKERPPPVPNPDYEPIRKGQRDLYSGLNQRRI

SEQ ID NO: 28 - protein, 50 aa, Homo sapiens
KNRKAKAKPVTRGAGAGGRQRGQNKERPPPVPNPDYEPIRKGQRDLYSGL

SEQ ID NO: 29 - DNA, 75 bp, Homo sapiens
  source 1..75: mol_type = genomic DNA
gctagcgccaccatggctctgcctgtgacagctctgctgctgcctctggccctgctgctccatgctgctagacct

SEQ ID NO: 30 - DNA, 63 bp, Homo sapiens
  source 1..63: mol_type = genomic DNA
atggctctgcctgtgacagctctgctgctgcctctggccctgctgctccatgctgctagacct

SEQ ID NO: 31 - DNA, 69 bp, Homo sapiens
  source 1..69: mol_type = genomic DNA
ggatccatggagtttggcctgagctggctgttcctggtggccatcctcaagggcgtgcagtgctccagg

SEQ ID NO: 32 - DNA, 63 bp, Homo sapiens
  source 1..63: mol_type = genomic DNA
atggagtttggcctgagctggctgttcctggtggccatcctcaagggcgtgcagtgctccagg

SEQ ID NO: 33 - DNA, 141 bp, Homo sapiens
  source 1..141: mol_type = genomic DNA
ctcgagaccaccacccccgcccctaggcctcccacacctgcccccacaatcgcctcccagcctctcagcctgaggcctgaagcttgcaggcccgctgccggaggagctgtccataccaggggactcgacttcgcctgcgac

SEQ ID NO: 34 - DNA, 135 bp, Homo sapiens
  source 1..135: mol_type = genomic DNA
accaccacccccgcccctaggcctcccacacctgcccccacaatcgcctcccagcctctcagcctgaggcctgaagcttgcaggcccgctgccggaggagctgtccataccaggggactcgacttcgcctgcgac

SEQ ID NO: 35 - DNA, 123 bp, Homo sapiens
  source 1..123: mol_type = genomic DNA
tctagaatcgaagtgatgtaccctccaccttacctggacaacgagaagtccaacggcaccatcatccacgtgaagggcaagcacctgtgtccttctccactgttccccggacctagcaagcct

SEQ ID NO: 36 - DNA, 117 bp, Homo sapiens
  source 1..117: mol_type = genomic DNA
atcgaagtgatgtaccctccaccttacctggacaacgagaagtccaacggcaccatcatccacgtgaagggcaagcacctgtgtccttctccactgttccccggacctagcaagcct

SEQ ID NO: 37 - DNA, 696 bp, Homo sapiens
  source 1..696: mol_type = genomic DNA
gagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 38 - DNA, 36 bp, Homo sapiens
  source 1..36: mol_type = genomic DNA
gagcccaagagctgcgacaagacccacacctgcccc

SEQ ID NO: 39 - DNA, 687 bp, Homo sapiens
  source 1..687: mol_type = genomic DNA
gagagcaagtacggccccccctgccccagctgccccgcccccgagttcctgggcggccccagcgtgttcctgttcccccccaagcccaaggacaccctgatgatcagccggacccccgaggtgacctgcgtggtggtggacgtgagccaggaggaccccgaggtgcagttcaactggtacgtggacggcgtggaggtgcacaacgccaagaccaagccccgggaggagcagttcaacagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccggctgaccgtggacaagagccggtggcaggagggcaacgtgttcagctgcagcgtgatgcacgaggccctgcacaaccactacacccagaagagcctgagcctgagcctgggcaag

SEQ ID NO: 40 - DNA, 27 bp, Homo sapiens
  source 1..27: mol_type = genomic DNA
gagagcaagtacggccccccctgcccc

SEQ ID NO: 41 - DNA, 72 bp, Homo sapiens
  source 1..72: mol_type = genomic DNA
atttacatttgggcccctctggctggaacctgcggagtcctgctgctgtccctggtgatcacactgtactgt

SEQ ID NO: 42 - DNA, 81 bp, Homo sapiens
  source 1..81: mol_type = genomic DNA
ttctgggtgctcgttgttgttggcggcgtgctggcctgttacagcctgctggttaccgtggccttcatcatcttttgggtg

SEQ ID NO: 43 - DNA, 78 bp, Homo sapiens
  source 1..78: mol_type = genomic DNA
gtgatgagcgtggccaccatcgtgatcgtggacatctgtatcaccggcggactgctgctgctggtgtactactggtcc

SEQ ID NO: 44 - DNA, 123 bp, Homo sapiens
  source 1..123: mol_type = genomic DNA
cgaagcaagcggagccggggaggacacagcgactacatgaacatgacccctcggaggccaggccccaccagaaagcactaccagccctacgcccctccccgggactttgccgcctatcggagc

SEQ ID NO: 45 - DNA, 123 bp, Homo sapiens
  source 1..123: mol_type = genomic DNA
cgaagcaagcggagccggctgctgcacagcgactacatgaacatgacccctcggaggccaggccccaccagaaagcactaccagccctacgcccctccccgggactttgccgcctatcggagc

SEQ ID NO: 46 - DNA, 126 bp, Homo sapiens
  source 1..126: mol_type = genomic DNA
aagaggggcagaaagaagctgctctacatcttcaagcagccctttatgagacccgtgcagacaacccaggaggaagacggatgcagctgcaggttccctgaggaggaggagggcggctgcgaactg

SEQ ID NO: 47 - DNA, 72 bp, Homo sapiens
  source 1..72: mol_type = genomic DNA
ctgtgcgcccggccccggcggagccccgcccaggaggacggcaaggtgtacatcaacatgcccggccggggc

SEQ ID NO: 48 - DNA, 156 bp, Homo sapiens
  source 1..156: mol_type = genomic DNA
tacttcctgggccggctggtgccccggggccggggcgccgccgaggccgccacccggaagcagcggatcaccgagaccgagagcccctaccaggagctgcagggccagcggagcgacgtgtacagcgacctgaacacccagcggccctactacaag

SEQ ID NO: 49 - DNA, 360 bp, Homo sapiens
  source 1..360: mol_type = genomic DNA
tggcggcggaagcggaaggagaagcagagcgagaccagccccaaggagttcctgaccatctacgaggacgtgaaggacctgaagacccggcggaaccacgagcaggagcagaccttccccggcggcggcagcaccatctacagcatgatccagagccagagcagcgcccccaccagccaggagcccgcctacaccctgtacagcctgatccagcccagccggaagagcggcagccggaagcggaaccacagccccagcttcaacagcaccatctacgaggtgatcggcaagagccagcccaaggcccagaaccccgcccggctgagccggaaggagctggagaacttcgacgtgtacagc

SEQ ID NO: 50 - DNA, 126 bp, Homo sapiens
  source 1..126: mol_type = genomic DNA
gccctgtacctgctgcggcgggaccagcggctgccccccgacgcccacaagccccccggcggcggcagcttccggacccccatccaggaggagcaggccgacgcccacagcaccctggccaagatc

SEQ ID NO: 51 - DNA, 147 bp, Homo sapiens
  source 1..147: mol_type = genomic DNA
caccagcggcggaagtaccggagcaacaagggcgagagccccgtggagcccgccgagccctgccactacagctgcccccgggaggaggagggcagcaccatccccatccaggaggactaccggaagcccgagcccgcctgcagcccc

SEQ ID NO: 52 - DNA, 144 bp, Homo sapiens
  source 1..144: mol_type = genomic DNA
cagcggcggaagtaccggagcaacaagggcgagagccccgtggagcccgccgagccctgccactacagctgcccccgggaggaggagggcagcaccatccccatccaggaggactaccggaagcccgagcccgcctgcagcccc

SEQ ID NO: 53 - DNA, 342 bp, Homo sapiens
  source 1..342: mol_type = genomic DNA
gatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctaga

SEQ ID NO: 54 - DNA, 336 bp, Homo sapiens
  source 1..336: mol_type = genomic DNA
agggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctaga

SEQ ID NO: 55 - DNA, 150 bp, Homo sapiens
  source 1..150: mol_type = genomic DNA
aagaaccggaaggccaaggccaagccagtgacaagaggtgctggtgctggcggcagacagagaggccagaacaaagaaagacctcctcctgtgcctaatcctgactacgagcccatccggaagggccagagagatctgtatagcggcctc

SEQ ID NO: 56 - DNA, 165 bp, Homo sapiens
  source 1..165: mol_type = genomic DNA
aagaaccggaaggccaaggccaagccagtgacaagaggtgctggtgctggcggcagacagagaggccagaacaaagaaagacctcctcctgtgcctaatcctgactacgagcccatccggaagggccagagagatctgtatagcggcctcaaccagagaagaatc

SEQ ID NO: 57 - protein, 488 aa, synthetic construct
  REGION 1..488: note = anti-CD22 CAR-2G-22BBz-1 (CAR1-1)
ASATMALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 1464 bp, synthetic construct
  misc_feature 1..1464: note = anti-CD22CAR-2G-22BBz-1-NT
  source 1..1464: mol_type = other DNA
gctagcgccaccatggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagctcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctaga

SEQ ID NO: 59 - protein, 381 aa, synthetic construct
  REGION 1..381: note = anti-CD19 CAR (CAR2-1)
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSRIEVMYPPPYLDNEKSNGTIIHVKGKHLCPSPLFPGPSKPVMSVATIVIVDICITGGLLLLVYYWSKNRKAKAKPVTRGAGAGGRQRGQNKERPPPVPNPDYEPIRKGQRDLYSGL

SEQ ID NO: 60 - DNA, 1146 bp, synthetic construct
  misc_feature 1..1146: note = anti-CD19 CAR (CAR2-1)-NT
  source 1..1146: mol_type = other DNA
atggagtttggcctgagctggctgttcctggtggccatcctcaagggcgtgcagtgctccagggacatccagatgacccagaccacaagcagcctgagcgcttccctcggcgacagggtgaccatctcctgtagagcctcccaagacatctccaagtacctgaactggtaccagcagaaacccgacggcaccgtgaagctgctgatctaccacaccagcaggctgcattccggcgtgccctccagattttccggcagcggctctggtaccgactacagcctcaccatcagcaacttagaacaggaggacatcgccacatatttctgccaacagggaaacacactcccctataccttcggcggcggcacaaagttagaaatcaccggctccacatccggcagcggaaaacctggttctggcgagggcagcaccaagggcgaagtgaagctgcaggaaagcggacctggactggtcgctcccagccagagcctcagcgtgacctgtacagtgagcggcgtgagcctgcctgattacggcgtgagctggattagacagcctcccaggaagggcttagaatggctcggcgtgatttggggcagcgagacaacctactataacagcgccctgaagagcaggctcaccattatcaaggacaacagcaaatcccaggtcttcctgaagatgaacagcctccagaccgacgacaccgccatctactactgcgccaagcactactattatggcggctcctacgccatggactactggggccagggcaccagcgtgacagtgtctagaatcgaagtgatgtaccctccaccttacctggacaacgagaagtccaacggcaccatcatccacgtgaagggcaagcacctgtgtccttctccactgttccccggacctagcaagcctgtgatgagcgtggccaccatcgtgatcgtggacatctgtatcaccggcggactgctgctgctggtgtactactggtccaagaaccggaaggccaaggccaagccagtgacaagaggtgctggtgctggcggcagacagagaggccagaacaaagaaagacctcctcctgtgcctaatcctgactacgagcccatccggaagggccagagagatctgtatagcggcctctga

SEQ ID NO: 61 - protein, 22 aa, synthetic construct
  REGION 1..22: note = P2A-1
GSGATNFSLLKQAGDVEENPGP

SEQ ID NO: 62 - protein, 22 aa, synthetic construct
  REGION 1..22: note = P2A-2
GSGATNFSLLKQAGDVEENPGP

SEQ ID NO: 63 - protein, 21 aa, synthetic construct
  REGION 1..21: note = T2A
GSGEGRGSLLTCGDVEENPGP

SEQ ID NO: 64 - protein, 23 aa, synthetic construct
  REGION 1..23: note = E2A
GSGQCTNYALLKLAGDVESNPGP

SEQ ID NO: 65 - protein, 25 aa, synthetic construct
  REGION 1..25: note = F2A
GSGVKQTLNFDLLKLAGDVESNPGP

SEQ ID NO: 66 - protein, 12 aa, synthetic construct
  REGION 1..12: note = furin cleavage peptide
RKRRGSGTPDPW

SEQ ID NO: 67 - protein, 34 aa, synthetic construct
  REGION 1..34: note = self-cleaving peptide sequence
RKRRGSGTPDPWGSGATNFSLLKQAGDVEENPGP

SEQ ID NO: 68 - protein, 905 aa, synthetic construct
  REGION 1..905: note = bicistronic 22x19CD3e CAR-1
ASATMALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSRIEVMYPPPYLDNEKSNGTIIHVKGKHLCPSPLFPGPSKPVMSVATIVIVDICITGGLLLLVYYWSKNRKAKAKPVTRGAGAGGRQRGQNKERPPPVPNPDYEPIRKGQRDLYSGL

SEQ ID NO: 69 - DNA, 2715 bp, synthetic construct
  misc_feature 1..2715: note = bicistronic 22x19CD3e CAR-1-NT
  source 1..2715: mol_type = other DNA
gctagcgccaccatggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagctcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccggatccatggagtttggcctgagctggctgttcctggtggccatcctcaagggcgtgcagtgctccagggacatccagatgacccagaccacaagcagcctgagcgcttccctcggcgacagggtgaccatctcctgtagagcctcccaagacatctccaagtacctgaactggtaccagcagaaacccgacggcaccgtgaagctgctgatctaccacaccagcaggctgcattccggcgtgccctccagattttccggcagcggctctggtaccgactacagcctcaccatcagcaacttagaacaggaggacatcgccacatatttctgccaacagggaaacacactcccctataccttcggcggcggcacaaagttagaaatcaccggctccacatccggcagcggaaaacctggttctggcgagggcagcaccaagggcgaagtgaagctgcaggaaagcggacctggactggtcgctcccagccagagcctcagcgtgacctgtacagtgagcggcgtgagcctgcctgattacggcgtgagctggattagacagcctcccaggaagggcttagaatggctcggcgtgatttggggcagcgagacaacctactataacagcgccctgaagagcaggctcaccattatcaaggacaacagcaaatcccaggtcttcctgaagatgaacagcctccagaccgacgacaccgccatctactactgcgccaagcactactattatggcggctcctacgccatggactactggggccagggcaccagcgtgacagtgtctagaatcgaagtgatgtaccctccaccttacctggacaacgagaagtccaacggcaccatcatccacgtgaagggcaagcacctgtgtccttctccactgttccccggacctagcaagcctgtgatgagcgtggccaccatcgtgatcgtggacatctgtatcaccggcggactgctgctgctggtgtactactggtccaagaaccggaaggccaaggccaagccagtgacaagaggtgctggtgctggcggcagacagagaggccagaacaaagaaagacctcctcctgtgcctaatcctgactacgagcccatccggaagggccagagagatctgtatagcggcctc

SEQ ID NO: 70 - protein, 124 aa, synthetic construct
  REGION 1..124: note = m971 antibody-VH
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 71 - protein, 107 aa, synthetic construct
  REGION 1..107: note = m971 antibody-VL
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 72 - protein, 10 aa, synthetic construct
  REGION 1..10: note = m971-CDRH1
GDSVSSNSAA

SEQ ID NO: 73 - protein, 9 aa, synthetic construct
  REGION 1..9: note = m971-CDRH2
TYYRSKWYN

SEQ ID NO: 74 - protein, 14 aa, synthetic construct
  REGION 1..14: note = m971-CDRH3
AREVTGDLEDAFDI

SEQ ID NO: 75 - protein, 6 aa, synthetic construct
  REGION 1..6: note = m971-CDRL1
QTIWSY

SEQ ID NO: 76 - skipped

SEQ ID NO: 77 - protein, 9 aa, synthetic construct
  REGION 1..9: note = m971-CDRL3
QQSYSIPQT

SEQ ID NO: 78 - protein, 118 aa, synthetic construct
  REGION 1..118: note = FMC63 antibody-VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTV

SEQ ID NO: 79 - protein, 107 aa, synthetic construct
  REGION 1..107: note = FMC63 antibody-VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 80 - protein, 8 aa, synthetic construct
  REGION 1..8: note = FMC63-CDRH1
GVSLPDYG

SEQ ID NO: 81 - protein, 7 aa, synthetic construct
  REGION 1..7: note = FMC63-CDRH2
IWGSETT

SEQ ID NO: 82 - protein, 14 aa, synthetic construct
  REGION 1..14: note = FMC63-CDRH3
AKHYYYGGSYAMDY

SEQ ID NO: 83 - protein, 6 aa, synthetic construct
  REGION 1..6: note = FMC63-CDRL1
QDISKY

SEQ ID NO: 84 - skipped

SEQ ID NO: 85 - protein, 9 aa, synthetic construct
  REGION 1..9: note = FMC63-CDRL3
QQGNTLPYT

SEQ ID NO: 86 - protein, 484 aa, synthetic construct
  REGION 1..484: note = anti-CD22 CAR-2G-22BBz-2 (CAR1-2)
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - DNA, 1452 bp, synthetic construct
  misc_feature 1..1452: note = anti-CD22 CAR-2G-22BBz-2-NT
  source 1..1452: mol_type = other DNA
atggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagctcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctaga

SEQ ID NO: 88 - protein, 901 aa, synthetic construct
  REGION 1..901: note = bicistronic 22x19CD3e CAR-2
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSRIEVMYPPPYLDNEKSNGTIIHVKGKHLCPSPLFPGPSKPVMSVATIVIVDICITGGLLLLVYYWSKNRKAKAKPVTRGAGAGGRQRGQNKERPPPVPNPDYEPIRKGQRDLYSGL

SEQ ID NO: 89 - DNA, 2703 bp, synthetic construct
  misc_feature 1..2703: note = bicistronic 22x19CD3e CAR-2-NT
  source 1..2703: mol_type = other DNA
atggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagctcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccggatccatggagtttggcctgagctggctgttcctggtggccatcctcaagggcgtgcagtgctccagggacatccagatgacccagaccacaagcagcctgagcgcttccctcggcgacagggtgaccatctcctgtagagcctcccaagacatctccaagtacctgaactggtaccagcagaaacccgacggcaccgtgaagctgctgatctaccacaccagcaggctgcattccggcgtgccctccagattttccggcagcggctctggtaccgactacagcctcaccatcagcaacttagaacaggaggacatcgccacatatttctgccaacagggaaacacactcccctataccttcggcggcggcacaaagttagaaatcaccggctccacatccggcagcggaaaacctggttctggcgagggcagcaccaagggcgaagtgaagctgcaggaaagcggacctggactggtcgctcccagccagagcctcagcgtgacctgtacagtgagcggcgtgagcctgcctgattacggcgtgagctggattagacagcctcccaggaagggcttagaatggctcggcgtgatttggggcagcgagacaacctactataacagcgccctgaagagcaggctcaccattatcaaggacaacagcaaatcccaggtcttcctgaagatgaacagcctccagaccgacgacaccgccatctactactgcgccaagcactactattatggcggctcctacgccatggactactggggccagggcaccagcgtgacagtgtctagaatcgaagtgatgtaccctccaccttacctggacaacgagaagtccaacggcaccatcatccacgtgaagggcaagcacctgtgtccttctccactgttccccggacctagcaagcctgtgatgagcgtggccaccatcgtgatcgtggacatctgtatcaccggcggactgctgctgctggtgtactactggtccaagaaccggaaggccaaggccaagccagtgacaagaggtgctggtgctggcggcagacagagaggccagaacaaagaaagacctcctcctgtgcctaatcctgactacgagcccatccggaagggccagagagatctgtatagcggcctc

SEQ ID NO: 90 - protein, 385 aa, synthetic construct
  REGION 1..385: note = anti-CD22 CD3e CAR (CAR2-2)
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVKNRKAKAKPVTRGAGAGGRQRGQNKERPPPVPNPDYEPIRKGQRDLYSGL

SEQ ID NO: 91 - protein, 21 aa, Homo sapiens
FLVIIVILSALFLGTLACFCV

SEQ ID NO: 92 - DNA, 63 bp, Homo sapiens
  source 1..63: mol_type = genomic DNA
ttcctggtcatcatcgtgatcctgagcgccctgttcctgggcaccctggcctgtttctgcgtg

SEQ ID NO: 93 - protein, 929 aa, synthetic construct
  REGION 1..929: note = full length 22x22CD3e CAR-6-His-FLAG
MALPVTALLLPLALLLHAARPHHHHHHQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVKNRKAKAKPVTRGAGAGGRQRGQNKERPPPVPNPDYEPIRKGQRDLYSGL

SEQ ID NO: 94 - DNA, 2791 bp, synthetic construct
  misc_feature 1..2791: note = full length 22x22CD3e CAR-6-His-FLAG-NT
  source 1..2791: mol_type = other DNA
atggcactgccagtgactgcattactcttgccactcgcgctactgttacacgcagcacgtccacatcaccatcaccatcaccaagtccaattgcaacaaagcgggccgggcatggtgaaaccgagtcaaacgttatctcttacgtgtgcgatttcgggggatagtgtcagcagcaattcagtggcgtggaattggattcgccaatcgccgagtcgcgggttggagtggctcgggcgcacgtattatcgcagcacatggtataatgattatgcggtcagcatgaaaagccgcattacgattaatccggatacgaacaaaaatcaatttagcttacaattaaattccgtcacgccggaagatacagcggtctattattgtgcgcgcgaggtcacgggggatctcgaagacgcgtttgatatttgggggcaagggaccatggtgactgtcagctctggtggagggggcagtggaggtgggggatcgggaggtggtggcagtgatattcaaatgatccaaagtccatccagcctatccgcatctgtcggagatcgcgtaacgattacgtgccgcgcgagtcaaacgatttggagctatctgaactggtaccggcaacgcccgggcgaagcgccgaatctcttgatttacgcggcgtcctcattacagtcgggtgtcccgagccgctttagcggccgcggaagcggtacggattttacgttaaccattagcagcctccaggcggaagattttgcgacgtattactgtcaacagagctatagcattccgcagacgtttggtcagggcacgaaattggagattaaactcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccggatccatggctctgcctgtgacagctctgctgctgcctctggccctgctgctccatgctgctagacctgactacaaagacgatgacgacaagcaggtgcagctccagcagtctggcccaggaatggtcaagcctagccagaccctgagcctgacctgcgccatcagcggcgacagcgtgtcctctaacagcgtcgcctggaactggatcagacagagccccagcagaggcctggaatggctgggccggacctactaccggtccacgtggtacaacgactacgccgtgtccatgaagtcccggatcaccatcaaccccgacaccaacaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcggtggaggcggtagcggcggtggcggttccgacatccagatgatccagagccctagctccctgagcgccagcgtgggcgacagagtgaccatcacctgtcgggccagccagaccatctggtcctacctgaattggtatcggcagcggccaggcgaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagtctagaatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtcaagaaccggaaggccaaggccaagcctgtgacaagaggtgctggtgctggcggcagacagagaggccagaacaaagaaagacctcctcctgtgcctaatcctgactacgagcccatccggaagggccagagagatctgtattctggcctgtgag

SEQ ID NO: 95 - protein, 915 aa, synthetic construct
  REGION 1..915: note = full length 22x22CD3e CAR
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVKNRKAKAKPVTRGAGAGGRQRGQNKERPPPVPNPDYEPIRKGQRDLYSGL

SEQ ID NO: 96 - DNA, 2749 bp, synthetic construct
  misc_feature 1..2749: note = full length 22x22CD3e CAR-NT
  source 1..2749: mol_type = other DNA
atggcactgccagtgactgcattactcttgccactcgcgctactgttacacgcagcacgtccacaagtccaattgcaacaaagcgggccgggcatggtgaaaccgagtcaaacgttatctcttacgtgtgcgatttcgggggatagtgtcagcagcaattcagtggcgtggaattggattcgccaatcgccgagtcgcgggttggagtggctcgggcgcacgtattatcgcagcacatggtataatgattatgcggtcagcatgaaaagccgcattacgattaatccggatacgaacaaaaatcaatttagcttacaattaaattccgtcacgccggaagatacagcggtctattattgtgcgcgcgaggtcacgggggatctcgaagacgcgtttgatatttgggggcaagggaccatggtgactgtcagctctggtggagggggcagtggaggtgggggatcgggaggtggtggcagtgatattcaaatgatccaaagtccatccagcctatccgcatctgtcggagatcgcgtaacgattacgtgccgcgcgagtcaaacgatttggagctatctgaactggtaccggcaacgcccgggcgaagcgccgaatctcttgatttacgcggcgtcctcattacagtcgggtgtcccgagccgctttagcggccgcggaagcggtacggattttacgttaaccattagcagcctccaggcggaagattttgcgacgtattactgtcaacagagctatagcattccgcagacgtttggtcagggcacgaaattggagattaaactcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccggatccatggctctgcctgtgacagctctgctgctgcctctggccctgctgctccatgctgctagacctcaggtgcagctccagcagtctggcccaggaatggtcaagcctagccagaccctgagcctgacctgcgccatcagcggcgacagcgtgtcctctaacagcgtcgcctggaactggatcagacagagccccagcagaggcctggaatggctgggccggacctactaccggtccacgtggtacaacgactacgccgtgtccatgaagtcccggatcaccatcaaccccgacaccaacaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcggtggaggcggtagcggcggtggcggttccgacatccagatgatccagagccctagctccctgagcgccagcgtgggcgacagagtgaccatcacctgtcgggccagccagaccatctggtcctacctgaattggtatcggcagcggccaggcgaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagtctagaatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtcaagaaccggaaggccaaggccaagcctgtgacaagaggtgctggtgctggcggcagacagagaggccagaacaaagaaagacctcctcctgtgcctaatcctgactacgagcccatccggaagggccagagagatctgtattctggcctgtgag

SEQ ID NO: 97 - DNA, 1158 bp, synthetic construct
  misc_feature 1..1158: note = anti-CD22 CD3e CAR (CAR2-2)-NT
  source 1..1158: mol_type = other DNA
atggctctgcctgtgacagctctgctgctgcctctggccctgctgctccatgctgctagacctcaggtgcagctccagcagtctggcccaggaatggtcaagcctagccagaccctgagcctgacctgcgccatcagcggcgacagcgtgtcctctaacagcgtcgcctggaactggatcagacagagccccagcagaggcctggaatggctgggccggacctactaccggtccacgtggtacaacgactacgccgtgtccatgaagtcccggatcaccatcaaccccgacaccaacaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcggtggaggcggtagcggcggtggcggttccgacatccagatgatccagagccctagctccctgagcgccagcgtgggcgacagagtgaccatcacctgtcgggccagccagaccatctggtcctacctgaattggtatcggcagcggccaggcgaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagtctagaatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtcaagaaccggaaggccaaggccaagcctgtgacaagaggtgctggtgctggcggcagacagagaggccagaacaaagaaagacctcctcctgtgcctaatcctgactacgagcccatccggaagggccagagagatctgtattctggcctgtga

SEQ ID NO: 98 - protein, 494 aa, synthetic construct
  REGION 1..494: note = anti-CD22 CAR-2G-22BBz-3 (CAR1-3)
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - DNA, 1482 bp, synthetic construct
  misc_feature 1..1482: note = anti-CD22 CAR-2G-22BBz-3 (CAR1-3)-NT
  source 1..1482: mol_type = other DNA
atggcactgccagtgactgcattactcttgccactcgcgctactgttacacgcagcacgtccacaagtccaattgcaacaaagcgggccgggcatggtgaaaccgagtcaaacgttatctcttacgtgtgcgatttcgggggatagtgtcagcagcaattcagtggcgtggaattggattcgccaatcgccgagtcgcgggttggagtggctcgggcgcacgtattatcgcagcacatggtataatgattatgcggtcagcatgaaaagccgcattacgattaatccggatacgaacaaaaatcaatttagcttacaattaaattccgtcacgccggaagatacagcggtctattattgtgcgcgcgaggtcacgggggatctcgaagacgcgtttgatatttgggggcaagggaccatggtgactgtcagctctggtggagggggcagtggaggtgggggatcgggaggtggtggcagtgatattcaaatgatccaaagtccatccagcctatccgcatctgtcggagatcgcgtaacgattacgtgccgcgcgagtcaaacgatttggagctatctgaactggtaccggcaacgcccgggcgaagcgccgaatctcttgatttacgcggcgtcctcattacagtcgggtgtcccgagccgctttagcggccgcggaagcggtacggattttacgttaaccattagcagcctccaggcggaagattttgcgacgtattactgtcaacagagctatagcattccgcagacgtttggtcagggcacgaaattggagattaaactcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctaga

SEQ ID NO: 100 - protein, 500 aa, synthetic construct
  REGION 1..500: note = anti-CD22 CAR-2G-22BBz-4 (CAR1-4)
MALPVTALLLPLALLLHAARPHHHHHHQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - DNA, 1502 bp, synthetic construct
  misc_feature 1..1502: note = anti-CD22 CAR-2G-22BBz-4 (CAR1-4)-NT
  source 1..1502: mol_type = other DNA
atggcactgccagtgactgcattactcttgccactcgcgctactgttacacgcagcacgtccacatcaccatcaccatcaccaagtccaattgcaacaaagcgggccgggcatggtgaaaccgagtcaaacgttatctcttacgtgtgcgatttcgggggatagtgtcagcagcaattcagtggcgtggaattggattcgccaatcgccgagtcgcgggttggagtggctcgggcgcacgtattatcgcagcacatggtataatgattatgcggtcagcatgaaaagccgcattacgattaatccggatacgaacaaaaatcaatttagcttacaattaaattccgtcacgccggaagatacagcggtctattattgtgcgcgcgaggtcacgggggatctcgaagacgcgtttgatatttgggggcaagggaccatggtgactgtcagctctggtggagggggcagtggaggtgggggatcgggaggtggtggcagtgatattcaaatgatccaaagtccatccagcctatccgcatctgtcggagatcgcgtaacgattacgtgccgcgcgagtcaaacgatttggagctatctgaactggtaccggcaacgcccgggcgaagcgccgaatctcttgatttacgcggcgtcctcattacagtcgggtgtcccgagccgctttagcggccgcggaagcggtacggattttacgttaaccattagcagcctccaggcggaagattttgcgacgtattactgtcaacagagctatagcattccgcagacgtttggtcagggcacgaaattggagattaaactcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaag

SEQ ID NO: 102 - DNA, 1158 bp, synthetic construct; identical to SEQ ID NO: 97
  misc_feature 1..1158: note = anti-CD22 CD3e CAR (CAR2-2)-NT
  source 1..1158: mol_type = other DNA

SEQ ID NO: 103 - protein, 393 aa, synthetic construct
  REGION 1..393: note = anti-CD22 CD3e CAR (CAR2-3) with FLAG
MALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVKNRKAKAKPVTRGAGAGGRQRGQNKERPPPVPNPDYEPIRKGQRDLYSGL

SEQ ID NO: 104 - DNA, 1182 bp, synthetic construct
  misc_feature 1..1182: note = anti-CD22 CD3e CAR (CAR2-3) with FLAG-NT
  source 1..1182: mol_type = other DNA
atggctctgcctgtgacagctctgctgctgcctctggccctgctgctccatgctgctagacctgactacaaagacgatgacgacaagcaggtgcagctccagcagtctggcccaggaatggtcaagcctagccagaccctgagcctgacctgcgccatcagcggcgacagcgtgtcctctaacagcgtcgcctggaactggatcagacagagccccagcagaggcctggaatggctgggccggacctactaccggtccacgtggtacaacgactacgccgtgtccatgaagtcccggatcaccatcaaccccgacaccaacaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcggtggaggcggtagcggcggtggcggttccgacatccagatgatccagagccctagctccctgagcgccagcgtgggcgacagagtgaccatcacctgtcgggccagccagaccatctggtcctacctgaattggtatcggcagcggccaggcgaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagtctagaatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtcaagaaccggaaggccaaggccaagcctgtgacaagaggtgctggtgctggcggcagacagagaggccagaacaaagaaagacctcctcctgtgcctaatcctgactacgagcccatccggaagggccagagagatctgtattctggcctgtga